(19) United States
(12) Patent Application Publication
(10) Pub. No.: US 2006/0210479 A1
(43) Pub. Date: Sep. 21, 2006

(54) Targeting chelants and chelates

(75) Inventors: Scott A. Young, Midland, MI (US); Louis R. DePalatis, Lake Jackson, TX (US); Garry E. Kiefer, Lake Jackson, TX (US)

Correspondence Address: KING & SPALDING LLP, 1180 PEACHTREE STREET, ATLANTA, GA, 30309, US

(73) Assignee: Dow Global Technologies Inc., Midland, MI (US)

(21) Appl. No.: 11/201,852

(22) Filed: Aug. 10, 2005

Related U.S. Application Data
(60) Provisional application No. 60/600,251, filed on Aug. 10, 2004.

(51) Int. Cl. A61K 49/10; C07F 5/00; A61K 31/555

(52) U.S. Cl. 424/9.363; 514/185; 540/465; 534/15

(57) ABSTRACT

Novel chelants and other compounds, and compositions thereof are provided, that are useful for detection and treatment of cancer and other abnormal and disease-state cells and tissues.

### CROSS REFERENCE TO RELATED APPLICATIONS

This application claims the benefit of U.S. Provisional Appl. No. 60/600,251, filed Aug. 10, 2004.

### FIELD OF THE INVENTION

The invention provides chelants, and chelates thereof for use as therapeutic agents, imaging agents and diagnostic agents. Acyclic and cyclic compounds containing alkyl phosphonic acid half esters are provided for use as therapeutic agents, for transporting agents into cells, and as diagnostic agents.

### DESCRIPTION OF RELATED ART

The use of lanthanide-based chelates (also known as contrast agents, or CAs) is established in both diagnostic and therapeutic medical applications. One of the most informative diagnostic modalities that rely heavily on the use of paramagnetic chelates to enhance image contrast is magnetic resonance imaging (MRI). In general, this class of metal based CAs does not have inherent targeting ability and relies on variations in soft tissue blood perfusion to augment contrast in regions of diminished or enhanced blood flow.

Metal-based chelates can also be site directed to specific epitopes of disease cells by covalent attachment to larger biotargeting molecules such as monoclonal antibodies. This approach allows site-specific delivery of the chelate to abnormal or diseased tissue and permits both diagnostic imaging and/or therapy, depending upon the choice of isotope. A shortcoming of this strategy involves increased complexity in which the targeting molecule constitutes most of the molecular structure, with the chelate being less than 10% of the overall molecular weight.

Aminocarboxylate and aminophosphonate chelating agents derived from 1,4,7,10-tetraazacyclododecane have been shown to form lanthanide chelates. See Cacheris, W. P., et al, Inorg. Chem. 26: pp. 958-960 (1987); and Simon, J., et al., U.S. Pat. No. 4,976,950.

Use of paramagnetic macrocyclic chelates based upon gadolinium (Gd) as contrast agents for magnetic resonance imaging has been described. Caravan, P. et al., Chem. Rev., 99: pp.2293-2352 (1999). This type of ligand can cause pronounced fluorescence when lanthanides such as terbium (Tb) and europium (Eu), are complexed at the central core. Kim et al., (Inorg. Chem. 34, 2233-2243 (1995)), have reported studies on some potential MRI contrast agents that are based upon macrocyclic pyridine-containing ligands having pendent carboxylic acids useful in forming stable lanthanide complexes.

The importance of macrocyclic lanthanide chelates for medical applications has also continued to grow with the development of tissue specific agents. Generally, applications have focused on the chelation of radioactive and paramagnetic metal ions for therapy and diagnosis (see, for example, U.S. Pat. No. 4,976,950; U.S. Patent Application Publication No. 2003/0118508; PCT WO 94/26755; and International Publication No. WO 03/035655A1). Examples of commercialized gadolinium chelates for MRI are Prohance™ by Squibb, and Dotarem™ by Guerbet. However, these molecules often do not have any fluorescent properties or the ability to target specific types of cells.

Commercial applications of fluorescent chelates have been primarily labeling of proteins and antibodies for immunoassays. Diamandis, E. P., et al., Clinica Chimica Acta, 194: pp. 19-50 (1990); and, U.S. Pat. No. 5,312,922. Products such as FIAgen™ (CyberFluor Inc., Toronto, Ontario, Canada) are available and utilize the europium chelate of 4,7-bis(chlorosulfonyl)-1,10-phenanthroline-2,9-dicarboxylic acid as the fluorescent label. Fluorescent labels of this type are extremely sensitive and can be detected in the subpicomolar range using time resolved fluorometry.

Griffin et al. (Tetrahedron Letters, 42: pp.3823-3825 (2001)) describes a lanthanide chelating ligand based on the cyclen (1,4,7,10-tetraazacyclododecane) nucleus which possesses a single carboxyl group for conjugation and two phosphonic acid pendant arms for lanthanide complexation. Chappell, et al. (Bioorg. Med. Chem., 7: pp. 2313-2320 (1999)) describes the synthesis of the bifunctional chelate PA-DOTA, and conversion to the isothiocyanato form followed by conjugation to the HuCC49 and HuCC49ΔCH₂ monoclonal antibodies and radiolabeling with ¹⁷⁷Lu.

U.S. Pat. Appl. Publ. No. 2003/0099598, published May 29, 2003, to The Dow Chemical Company, discloses fluorescent chelates of lanthanide, terbium, europium and dysprosium with tetraazamacrocyclic compounds for use as in vitro and in vivo diagnostic agents, that are tissue specific imaging agents for soft tissue cancers.

Parker et al. have described a series of tri-aza macrocycles (U.S. Pat. Nos. 5,247,075; 5,247,077; and 5,484,893) and tetra-aza macrocycles (U.S. Pat. Nos. 5,342,936 and 5,653,960) for use in diagnosis and therapy.

U.S. Pat. Nos. 5,462,725 and 5,834,456 to The Dow Chemical Company describe 2-pyridylmethylenepolyazamacrocyclo-phosphonic acid compounds complexed with Gd, Mn or Fe ions for use in diagnostic applications. U.S. Pat. No. 5,714,604 to The Dow Chemical Company discloses processes for preparing azamacrocyclic compounds. U.S. Pat. No. 5,750,660, issued May 12, 1998 to The Dow Chemical Company describes the preparation of bicyclopolyazamacrocyclophosphonic acid half esters, complexes thereof with Gd, Mn or Fe ions, and their use as contrast agents.

J. C. Frias et al., Org. Biomol. Chem. (2003) 1:905-907 describes coordinated, cationic lanthanide complexes that have the ability to be taken up by mouse fibroblast (NIH 3T3) cells. This requires an aromatic moiety (heteroaromatic for DNA breakage) and utilizes amide group oxygens as coordinating groups for the lanthanide. However, this is not suitable for in vivo administration.

Schrader, J Inclusion Phenom. & Macrocycl. Chem. 34:117-29 (1999), describes that organic phosphonate groups, in coordination with a cation can function to permit selective attachment to guanidium groups. Manning et al. describes the conjugation of a trifunctional lanthanide chelate to a benzodiazepine receptor ligand and a cyclen-based fluorophore (Organic Letters, Vol. 4: pp.1075-1078 (2002)). The described contrast agent is described as having bright luminescence and good MRI contrast characteristics. U.S. Patent Publ. No. 2003/0129579 to Bonhop et al. discloses polyazamacrocyclic compounds having phosphoester chains and light harvesting moieties.

U.S. Pat. Nos. 4,885,363, 5,474,756, and 6,143,274, describe non-ionic (charge-neutral) metal-chelated (e.g. gadolinium or radioactive nuclide) ligands for use as contrast agents in X-ray imaging, radionuclide imaging and ultrasound imaging. The compounds are also described as being useful in radiotherapy or imaging applications wherein the metal-chelating ligands are bound to a monoclonal antibody or a fragment thereof for disease-specific targeting.

Three derivatives of 1,4,7,10-tetraazacyclododecane-1,4,7,10-tetrakis(methylene phosphonic acid) (DOTP) containing a hydrophobic substituent on one side chain were prepared and their lanthanide (Yb and Tm) complexes analyzed by NMR (Li et al., Inorg. Chem., 40: pp.6572-6579 (2001)). U.S. Pat. No. 5,874,573, issued Feb. 23, 1999 to Concat, Inc. discloses compounds with chelation affinity for use in medical therapy.

A number of fluorescent chelates of terbium, europium, and dysprosium with tri- and tetra-aza macrocyclic compounds have been described for use as fluorescent in vitro or in vivo diagnostic and imaging agents (U.S. Pat. No. 5,928,627) or as tissue-specific imaging agents for soft tissue cancers (U.S. Patent Application Publication No.: 2003/0099598 A1).

WO 02/46147 describes compounds that selectively target perturbed membranes, where the compounds comprise a lipophilic group attached to a non-metal fluorophore, where the lipophilic group is an alkyl chain of C₁-C₆ and the fluorophore is organic. However, this provides no description of a molecule that transits the cell membrane or that targets any intracellular structures.

As is the case for molecules useful in most disease-related applications, it would be desirable for chelants and chelates intended for such uses, in vitro or in vivo, to exhibit at least a preference, more preferably a specificity or selectivity, for the disease cells or tissues involved in the intended therapy or diagnosis, vis-à-vis healthy cells and tissues. As noted, most traditional molecules useful for these applications provide this specificity as a result of their selective binding affinity for one or more diseased-cell surface molecule, such as a cell-surface glycoprotein. A classic example is the use of a diseased-cell surface molecule-specific antibody as a targeting agent to deliver to the diseased cell(s) a molecule that is covalently bound to the antibody.

Previous reports have shown that certain types of luminescent chelates possess the ability to target early stage cancer. See PCT WO 97/40055, published Oct. 30, 1997 which describes fluorescent chelates of terbium and europium with tri- and tetra-cyclopolyazamacrocyclic compounds as tissue specific diagnostic agents. PCT WO 03/035655, PCT WO 03/035114, U.S. Pat. No. 5,928,627, and published patent applications US 2003-0133872 and US 2003-0099598 describe targeting chelate structures that function as tissue-specific diagnostic agents and radioisotope delivery agents for soft tissue tumors and cancers. PCT WO 03/035114 to Dow Global Technologies, published May 1, 2003, discloses the treatment of disease states, particularly, epithelial cancer or cancer of the lymphatic system, with radioactive chelates. PCT WO 92/067999, published Sep. 6, 2002, discloses actinium complexes and for targeted radiotherapy.

There is a need for chelants and chelates that are useful for specific targeting of abnormal cells, in vitro or in vivo. Moreover, it would represent advancement in the art to elucidate the features and/or mechanism of action of targeting chelates so as to be able to extend the range of molecules and complexes that may be employed, as well as the uses for which they may be employed.

There is a need for small molecule diagnostic, imaging, and therapeutic agents, which can be specifically localized into a specific tissue or diseased cells, such as a perturbed cell, within a host without the need for attachment to expensive delivery molecules such as antibodies and antibody fragments. There is a particular need for methods and compositions for the treatment or diagnosis of cancer and diseases associated with apoptosis. It is an object of the invention to address these needs.

### SUMMARY OF THE INVENTION

The present invention provides novel chelants and chelates and their derivatives, which are useful for specific targeting of abnormal cells, ex-vivo, in vitro or in vivo, and may be readily employed for a variety of diagnostic and therapeutic purposes. The present invention further delineates features and/or mechanisms of action of targeting chelates, extends the range of useful molecules and complexes based thereon, and provides still further uses for which targeting chelates and other molecules and complexes may be employed. A variety of cyclic and acyclic compounds containing alkyl phosphonic acid half esters also are provided for use as therapeutic agents, for transporting agents into cells, and as diagnostic agents.

In one embodiment there is provided a compound of Formula (I) or (Ia) or a salt thereof: 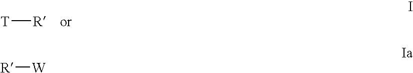
wherein T is 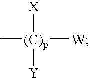
wherein p is 0, 1, 2, 3, 4, 5 or 6;
wherein each X and Y if present are independently H, OH, C₁-C₆ alkyl, substituted or unsubstituted aryl, or unsubstituted or substituted heterocycle;
wherein W is 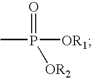
   wherein each R₁ and R₂ are independently H, C₁-C₁₀ linear or branched alkyl, C₂-C₁₀ linear or branched alkenyl, C₂-C₁₀ linear or branched alkynyl, trifluoromethyl, substituted or unsubstituted aryl, or substituted or unsubstituted heteroaryl; and
wherein R′ is: 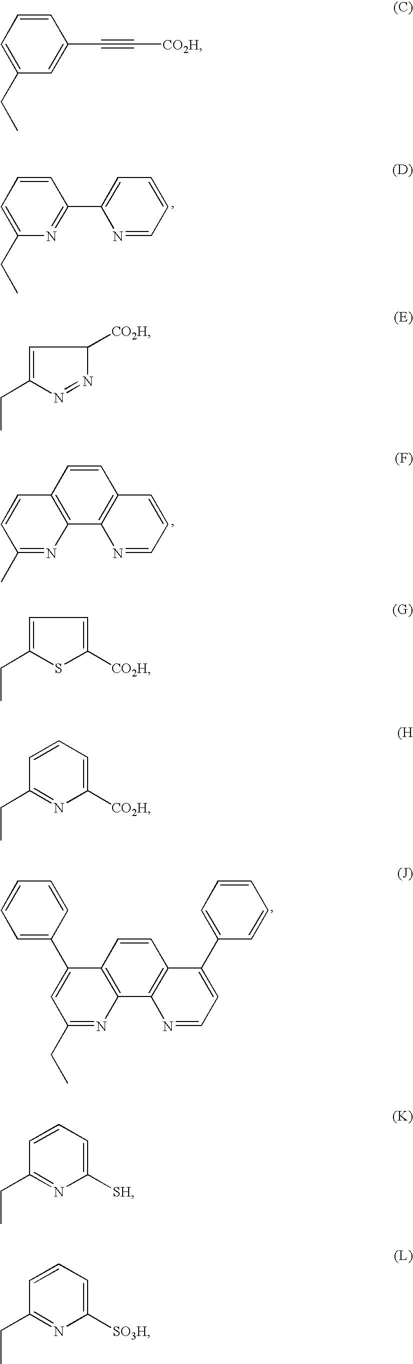 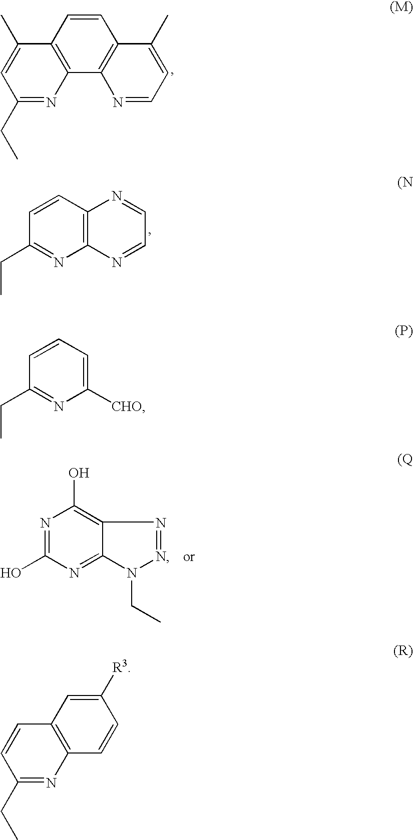
wherein R³ is H or C1-6 alkyl, e.g., methyl, ethyl, propyl or butyl, which is optionally substituted.

Also provided is a compound of Formula II or salt thereof: 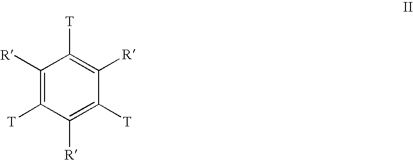
wherein each T is independently 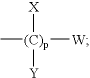
wherein p is 0, 1, 2, 3, 4, 5 or 6;
wherein each X and Y are independently H, OH, C₁-C₆ alkyl, substituted or unsubstituted aryl, or unsubstituted or substituted heterocycle;
wherein W is 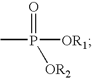
   wherein each R₁ and R₂ are independently H, C₁-C₁₀ linear or branched alkyl, C₂-C₁₀ linear or branched alkenyl, C₂-C₁₀ linear or branched alkynyl, trifluoromethyl, substituted or unsubstituted aryl, or substituted or unsubstituted heteroaryl; and
wherein each R′ is independently H, C₁-6 alkyl that is optionally substituted.

In one embodiment in the compound of Formula I, Ia or II, at least one of R₁ and R₂ is H. In another embodiment of a compound of Formula I, Ia or II, one of R₁ and R₂ is H and the other is alkyl, e.g., methyl, ethyl, n-propyl, isopropyl, n-butyl, sec-butyl, isobutyl, tertbutyl or cyclobutyl.

In another embodiment, there is provided a complex of a compound of Formula I, Ia or II and a metal cation.

In a further embodiment, there is provided a non-covalent or covalent conjugate of a compound of Formula I, Ia or II and a therapeutic agent, such as an anti-cancer agent.

Also provided are pharmaceutically acceptable compositions comprising a compound of Formula I, Ia or II, or a salt thereof, or a covalent or non-covalent complex thereof and a pharmaceutically acceptable carrier.

In yet another embodiment, a method for the diagnosis or treatment of a disease state in a host is provided comprising administering to the host an effective amount a compound disclosed herein in a pharmaceutically acceptable carrier. Such hosts include mammals, including humans.

Further provided is a method for the diagnosis and/or treatment of a disease state in a host comprising administering an effective amount of a compound disclosed herein, wherein the molecule is optionally non-covalently or covalently conjugated to a therapeutic agent, wherein the therapeutic agent is optionally an anti-cancer agent. The compound is optionally in the form of a chelant associated with a cation (“chelate”). In one embodiment, the compound is a compound of Formula I, Ia or II.

In another embodiment, the compound is for example PCTMB or QCTME or another polyazamacrocyclic molecule as described herein.

The disease state is e.g., epithelial cancer or cancer of the lymphatic system.

Also provided is an isolated non-covalent complex of Tb-PCTMB and a polypeptide having an amino acid sequence of SEQ ID NO: 2. Further provided is a complex of Tb-PCTMB and a polypeptide having an amino acid sequence of SEQ ID NO: 2, wherein the Tb-PCTMB is non-covalently bound as a dimer to the polypeptide.

Also provided is a method of diagnosing or treating a disease in a host comprising administering to a host or tissue or cell sample therefrom a molecule capable of binding to a protein having the sequence of SEQ. ID NO.:2.

In another embodiment, a method of evaluating the efficacy of a compound as a therapeutic or diagnostic agent is provided, the method comprising screening the compound for ability to bind to a protein of SEQ. ID No. 2 or a fragment thereof optionally having at least 20 amino acids.

Also provided is a method of treatment of a disease state in a host, the method comprising administering to the host an effective amount of a chelate comprising a cation complexed with a chelate, the chelate comprising a phosphonic half ester, wherein the cation is not a radionuclide.

### DESCRIPTION OF THE FIGURES

The following Figures form part of the present specification and are included to further demonstrate certain aspects of the present invention. The invention may be better understood by reference to one or more of these figures in combination with the detailed description of specific embodiments presented herein.

FIG. 1 illustrates Scheme 1, depicting a representative pathway for synthesis of chelants disclosed herein including those of EuPCTMB, and for formation of chelates therefrom. Synthesis of the tris-(n-butyl)phosphonate ester “PCTMB” is shown, followed by complexation with Europium to form Eu-PCTMB. In a similar manner, the compound Tb-PCTMB, as contemplated herein, can be prepared.

FIG. 2 illustrates Scheme 2, depicting a representative pathway for synthesis of chelates Eu-QCTME. Synthesis of the tris-(ethyl)phosphonate ester “QCTME” is shown, followed by complexation with Europium to form Eu-QCTME.

FIG. 3 illustrates a representative pathway for synthesis of chelants 1-3, and includes the synthesis of the quinoline methyl, ethyl and n-butyl phosphonate half esters.

FIG. 4 illustrates a representative pathway for synthesis of chelants 4-6, and includes the synthesis of the pyridyl bis-methyl, ethyl and n-butyl phosphonate half esters

FIG. 5 illustrates a representative pathway for synthesis of chelants 7-12. Synthesis of the 1,3,5 benzyl-tris methyl, ethyl and n-butyl phosphonate half esters.

FIG. 6 illustrates a representative pathway for synthesis of chelants 13-18, and includes the synthesis of 1,3,5-N alkyl benzene compounds.

FIG. 7 illustrates a representative pathway for synthesis of chelants 19-21, and includes the synthesis of phosphonate alkyl half esters and of simple alkylated derivatives of spermidine.

FIG. 8 illustrates a representative pathway for the synthesis of chelants 22-24, where the synthesis of phosphonate alkyl half esters and simple alkylated derivatives of spermidine is shown.

FIG. 9 represents structures of representative compounds of Formula I, Ia or II.

FIG. 10 presents dose response and time course curves for each cell line incubated with Tb-PCTMB. FIG. 10A presents dose response curves, for each identified cell line, that represent fluorescence detected from cell-bound and cell-contained Tb-PCTMB (counts per mm²), as determined for varying concentrations of Tb-PCTMB administered (0, 1 μM, 0.5 mM, 1.0 mM, and 2.0 mM), measured at 2 h post-administration. FIG. 10B presents time course curves, for each identified cell line, that represent fluorescence detected from chelates bound to or contained by cells treated with 1.0 mM of Tb-PCTMB (counts per mm²), as determined at varying times following administration of Tb-PCTMB (0, 1, 2, 4, and 8 h). Four cell lines (LNCaP, Caco-2, RBL-2H3 and PZ-HVP-7) were analyzed.

FIG. 11 presents dose response and time course curves for each cell line incubated with Eu-QCTME. FIG. 11A presents dose response curves for each identified cell line, that represent fluorescence detected from cell-bound and cell-contained Eu-QCTME (counts per mm2), as determined for varying concentrations of Eu-QCTME administered (0, 1 μM, 0.5 mM, 1.0 mM, and 2.0 mM), measured at 2 h post-administration. FIG. 11B presents time course curves, for each identified cell line, that represent fluorescence detected from chelates bound to or contained by cells treated with 1.0 mM of Eu-QCTME (counts per mm²), as determined at varying times following administration of Eu-QCTME (0, 1, 2, 4, and 8 h). Four cell lines (LNCaP, Caco-2, RBL-2H3 and PZ-HVP-7) were analyzed.

FIG. 12 presents dose response curves for both untreated HEK293 cells (normal, HEK293) and apoptosis-induced HEK293 cells (apoptotic, aHEK293), during incubation with targeting chelates for 2 hours. FIG. 12A presents results for fluorescence (counts per mm²) from chelates found specifically associated with the cell membrane and cell cytoplasm fractions; Eu-QCTME and closely related chelate structures were tested. FIG. 12B presents results for fluorescence (counts per mm²) from chelates found specifically associated with the cell membrane and cell cytoplasm fractions; Tb-PCTMB and closely related chelate structures were tested.

FIG. 13 presents a graph of binding kinetics of EuQCTME to cancer cells, CaCo-2, Du-145, SK-MES, HLaC, and C33-A, and to non-cancer cells, NCM-460. Results are normalized to control (NCM-460) and represent averages of triplicate samples measured as chelate fluorescence per well remaining after gentle washing of attached cells to remove unbound chelate.

FIG. 14 presents graphs showing the kinetics of cytoplasmic (▪) and nuclear (▴) uptake of EuQCTME to malignant and non-malignant cells: 14A. NCM460; 14B. Caco-2; 14C. DU-145; 14D. SKMES; 14E. HLAC; 14F. C33-A.

FIG. 15 presents cell inhibition and cytotoxicity assay results for HT-29 colon adenocarcinoma cells. FIGS. 15A-15B present comparative cytoxicity curves for EuQCTME (EuQM) and CPT-11, determined at 96 hours continuous exposure to these agents; the mean IC₅₀ values computed for these data are also shown. FIGS. 15C-15F present bar charts reporting concentration-dependent changes in 490 nm absorbance in the MTS assay, performed at 24, 48, 72, and 96 hours of exposure to EuQCTME; the data were normalized for the absorbance of controls lacking EuQCTME.

FIG. 16 presents cell inhibition and cytotoxicity assay results for HLAC head-and-neck squamous carcinoma cells. FIGS. 16A-16B present comparative cytoxicity curves for EuQCTME (EuQM) and cisplatin, determined at 96 hours continuous exposure to these agents; the mean IC₅₀ values computed for these data are also shown. FIGS. 16C-16F present bar charts reporting concentration-dependent changes in 490 nm absorbance in the MTS assay, performed at 24, 48, 72, and 96 hours of exposure to EuQCTME; the data were normalized for the absorbance of controls lacking EuQCTME.

FIG. 17 represents cell inhibition and cytotoxicity assay results for SK-MES lung non-small-cell squamous carcinoma cells. FIGS. 17A-17B present comparative cytoxicity curves for EuQCTME (EuQM) and cisplatin, determined at 96 hours continuous exposure to these agents; the mean IC₅₀ values computed for these data are also shown. FIGS. 17C-17F present bar charts reporting concentration-dependent changes in 490 nm absorbance in the MTS assay, performed at 24, 48, 72, and 96 hours of exposure to EuQCTME; the data were normalized for the absorbance of controls lacking EuQCTME.

FIG. 18 presents cell inhibition and cytotoxicity assay results for C33-A cervical carcinoma cells. FIGS. 18A-18B present comparative cytoxicity curves for EuQCTME (EuQM) and cisplatin, determined at 96 hours continuous exposure to these agents; the mean IC₅₀ values computed for these data are also shown. FIG. 18C presents a bar chart reporting concentration-dependent changes in 490 nm absorbance in the MTS assay, performed at 96 hours of exposure to EuQCTME; the data were normalized for the absorbance of controls lacking EuQCTME.

FIG. 19 presents cell inhibition and cytotoxicity assay results for LnCaP prostate adenocarcinoma cells. FIGS. 19A-19B present comparative cytoxicity curves for EuQCTME (EuQM) and mixantrone, determined at 96 hours continuous exposure to these agents; the mean IC₅₀ values computed for these data are also shown. FIGS. 19C-19F present bar charts reporting concentration-dependent changes in 490 nm absorbance in the MTS assay, performed at 24, 48, 72, and 96 hours of exposure to EuQCTME; the data were normalized for the absorbance of controls lacking EuQCTME.

FIG. 20 presents cell inhibition and cytotoxicity assay results for DU-145 prostate adenocarcinoma cells. FIGS. 20A-20B present comparative cytoxicity curves for EuQCTME (EuQM) and mixantrone, determined at 96 hours continuous exposure to these agents; the mean IC₅₀ values computed for these data are also shown. FIGS. 20C-20F present bar charts reporting concentration-dependent changes in 490 nm absorbance in the MTS assay, performed at 24, 48, 72, and 96 hours of exposure to EuQCTME; the data were normalized for the absorbance of controls lacking EuQCTME.

FIG. 21 presents cell inhibition and cytotoxicity assay results for MDA-231 breast adenocarcinoma cells. FIGS. 21A-21B present comparative cytoxicity curves for EuQCTME (EuQM) and paclitaxel, determined at 96 hours continuous exposure to these agents; the mean IC₅₀ values computed for these data are also shown. FIGS. 21C-21F present bar charts reporting concentration-dependent changes in 490 nm absorbance in the MTS assay, performed at 24, 48, 72, and 96 hours of exposure to EuQCTME; the data were normalized for the absorbance of controls lacking EuQCTME.

FIG. 22 presents cell inhibition and cytotoxicity assay results for MDA-231(M) breast adenocarcinoma cells. FIGS. 22A-22B present comparative cytoxicity curves for EuQCTME (EuQM) and paclitaxel, determined at 96 hours continuous exposure to these agents; the mean IC₅₀ values computed for these data are also shown. FIGS. 22C-22F present bar charts reporting concentration-dependent changes in 490 nm absorbance in the MTS assay, performed at 24, 48, 72, and 96 hours of exposure to EuQCTME; the data were normalized for the absorbance of controls lacking EuQCTME.

FIG. 23 presents cell inhibition and cytotoxicity assay results for Caki-1 renal, fast-growing carcinoma cells. FIGS. 23A-23B present comparative cytoxicity curves for EuQCTME (EuQM) and cytoxan, determined at 96 hours continuous exposure to these agents; the mean IC₅₀ values computed for these data are also shown. FIGS. 23C-23F present bar charts reporting concentration-dependent changes in 490 nm absorbance in the MTS assay, performed at 24, 48, 72, and 96 hours of exposure to EuQCTME; the data were normalized for the absorbance of controls lacking EuQCTME.

FIG. 24 presents cell inhibition and cytotoxicity assay results for Caco-2 colorectal adenocarcinoma cells. FIGS. 24A-24B present comparative cytoxicity curves for EuQCTME (EuQM) and CPT-11, determined at 96 hours continuous exposure to these agents; the mean IC₅₀ values computed for these data are also shown. FIGS. 24C-24F present bar charts reporting concentration-dependent changes in 490 nm absorbance in the MTS assay, performed at 24, 48, 72, and 96 hours of exposure to EuQCTME; the data were normalized for the absorbance of controls lacking EuQCTME.

FIG. 25 presents cell inhibition and cytotoxicity assay results for NCM-460 non-malignant cells (also called HMN-460, an immortalized, normal colon mucosal cell line). FIGS. 25A-25B present comparative cytoxicity curves for EuQCTME (EuQM) and CPT-11, determined at 96 hours continuous exposure to these agents; the mean IC₅₀ values computed for these data are also shown. FIG. 25C presents a bar chart reporting concentration-dependent changes in 490 nm absorbance in the MTS assay, performed at 96 hours of exposure to EuQCTME; the data were normalized for the absorbance of controls lacking EuQCTME.

FIG. 26 shows the structure of representative chelant and chelate compounds.

### DETAILED DESCRIPTION OF THE INVENTION

Molecules are provided that are useful for a wide range of diagnostic and therapeutic applications. The present invention provides a family of molecules that can be used to target early stage disease and other abnormal cells. In one embodiment, the compounds provided herein contain from 1 to 6 phosphonate groups or phosphonate ester groups, and are capable of specific targeting of disease-state cells and other abnormal cells, even at a very early stage of disease or abnormality.

In one embodiment, at least one or all of the phosphonates present therein are phosphonate esters, optionally each with an aliphatic ester partner. These molecules, chelants, and chelates, can exhibit membrane permeability toward abnormal and disease-state cells, as well as, in some embodiments, specific binding to intracellular proteins including endoplasmic proteins and cytoplasmic proteins.

The chelant in one embodiment is contacted with a metal or non-metal cation under conditions in which the chelant complexes with the cation, to form a chelate. The chelate can be administered to a subject to treat or detect an abnormal or disease-state cell or tissue. In one embodiment, a method of treatment of a patient afflicted with a disease characterized by diseased or perturbed cells is provided comprising administering to the patient in need of such treatment a therapeutically effective amount of a chelant or chelate complex thereof. Complexes of these chelants, in combination with cations as radioisotopes or paramagnetic cations, are particularly useful in diagnostic studies in nuclear medicine, in magnetic resonance imaging, or as specific targeting agents for abnormal, perturbed, or diseased cells both in vitro and in vivo for therapeutic use.

The use of the compounds, including chelants, advantageously permits an increase of the cellular residence time, or increase in the rate of uptake into abnormal or disease-state cells or tissues, or a combination thereof, of a metal or non-metal element complexed or conjugated to the chelant. Without being limited to any theory, in some embodiments, it is possible that diseased or perturbed cells, in particular cancer cells or apoptotic cells, have enhanced permeability for the chelants and chelates which enhances the specific targeting to these cells, and enhances their efficiacy for the treatment of disorders associated with these diseased or perturbed cells.

The therapeutically effective amount of the compound may be administered in one embodiment in the form of a pharmaceutical formulation comprising the compound and a suitable carrier. Such pharmaceutical formulations can also include flavors, binders, lubricants, inert diluents, lubricating, surface active or dispersing agents, and numerous other additives known in the art of pharmaceutical formulations.

I. Therapeutic and Diagnostic Applications

The compounds disclosed herein can be used for the treatment and diagnosis of diseases associated with abnormal cells, and in particular cells with perturbed membranes that have enhanced permeability to the compounds in comparison with normal cells. In this manner, the compound is targeted to and taken up specifically by the abnormal cell. As used herein, the term “abnormal cell” means a cell that exhibits either molecular or morphological differences from a corresponding healthy cell. Thus, “abnormal cells” include pre-disease state cells, disease-state cells, and so forth, for example, cancer cells, pre-cancerous cells, apoptotic cells, and pre-apoptotic cells. “Abnormal tissue” as used herein means a tissue that contains at least one abnormal cell.

The compounds can be used to treat or diagnose diseases and conditions associated with abnormal cells, including cancer. The compounds can be used to target cancerous, apoptotic, pre-cancerous, and pre-apoptotic cells and tissues. Soft tissue cancers and pre-cancerous soft-tissues are particularly susceptible of treatment and/or diagnosis thereby.

A variety of tumors can be treated or diagnosed. The compounds can be used to diagnose or treat carcinomas that originate from epithelial cells, sarcomas that originate from mesodermal (connective tissue) origin, and lymphomas from the lymphatic system. For example, colorectal adenocarcinomas and squamous cancer of the oral cavity can be diagnosed or treated. Other diseases that can be treated or diagnosed include leukemia and sickle cell anemia. In one embodiment, where the compound is administered to diagnose a tumor in a host, after surgical removal of the tumor, the resection margins are defined.

Other examples include leukemia (including acute leukemia (e.g., acute lymphocytic leukemia, acute myelocytic leukemia (including myeloblastic, promyelocytic, mylomonocytic, monocytic, and erythroleukemia)) and chronic leukemia (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, solid tumors, sarcomas and carcinomas such as fibrosarcoma, myxosarcoma, fiposarcoma, chondrosarcoma, osteogenic sarcoma, chordoma, anglosarcoma, endotheliosarcoma, lymphangiosarcoma, lymphangioendotheliosarcoma, synovioma, mesothelioma, Ewing's tumor, leiomyosarcoma, rhabdomyosarcoma, dermatofibrosarcoma, neurofibrosarcoma, colon carcinoma, pancreatic cancer, breast cancer, ovarian cancer, prostate cancer, squamous cell carcinoma, basal cell carcinoma, hepatocellular carcinoma, adenocortical carcinoma, melanoma, testicular carcinoma, esophageal carcinoma, and adenocarcinoma. In particular, the methods and compounds can be used to treat human colon, prostate, non-small cell lung carcinoma, head and neck carcinoma, cervical, renal and breast cancer.

Additional examples of carcinomas and sarcomas that can be treated with the compounds include, but are not limited to: malignant fibrous histiocytoma, liposarcomas, synovial sarcoma, transitional cell carcinoma of bladder, papillary carcinoma of thyroid, follicular carcinoma of thyroid, gastrinoma, pituitary adenoma, cervical carcinoma and thymic carcinoma.

Examples of lymphomas that can be treated or diagnosed with the compounds include the following: Burkitt's lymphoma; central nervous system (CNS) lymphoma; cutaneous T-cell lymphoma; Epstein-Barr Virus; Hodgkin's disease; anaplastic large cell lymphoma (ALCL); lymphoblastic lymphoma; lymphoplasmacytoid lymphoma; MALT/MALToma (mucosa-associated lymphoid tissue); marginal zone lymphoma; mycosis fimgoides; nasal T-cell lymphoma; follicular center cell lymphoma; T-cell lymphoma/leukemia; and small lymphocytic lymphoma.

Examples of pre-cancerous conditions that can be treated or diagnosed with the compounds include the following: lymphomatoid papulosis (LyP); solar or actinic keratosis; cervical dysplasia; bronchial lesions; epithelial lesions; cervical lesions; colon polyps; myelodysplastic syndrome (MDS); Li-Fraumeni syndrome (LFS), and precancerous moles.

The chelants may be chelated with a non-radioactive or radioactive metal for the treatment of a cancerous condition. Further, the compound can be conjugated with the appropriate therapeutic agent, such as an anti-cancer agent, to enhance the efficacy of the drug.

In a preferred embodiment, due to their specificity for abnormal cells, the compounds exhibit activity against cancer and other diseases in a patient and exhibit a minimal effect on normal cells in the patient.

In one embodiment, a method for the treatment of a disease state in a host is provided, comprising administering to the host an effective amount of a chelate, the chelate comprising a complex of chelant disclosed herein and a non-radioactive metal cation. The cation is e.g. a metal ion other than a radionuclide. Exemplary cations include rare earth metals, e.g., La, Ce, Pr, Nd, Pm, Sm, Eu, Gd, Tb, Dy, Ho, Er, Tm, Yb and Lu. The compounds of Formula I, Ia and II for example as described herein can be used. Other useful compounds include PCTMB and QCTME. In the method, for example, the disease state is epithelial cancer or cancer of the lymphatic system, including epithelial cancer in the skin, colon, oral cavity, or cervix. In another particular embodiment, the chelate selectively penetrates a perturbed cell membrane of diseased cells of the host in preference to normal cells, thus resulting in targeting to the diseased tissue or cells, such as tumor cells.

II. Compounds

In one embodiment, a compound of Formula (I) or (Ia) or a salt thereof is provided: 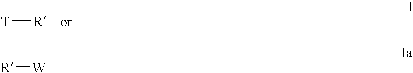
wherein each T is independently 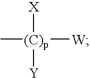
wherein p is 0, 1, 2, 3, 4, 5 or 6;
wherein each X and Y if present are independently H, OH, C₁-C₆ alkyl, substituted or unsubstituted aryl, or unsubstituted or substituted heterocycle;
wherein W is 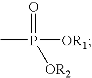
   wherein each R₁ and R₂ are independently H, C₁-C₁₀ linear or branched alkyl, C₂-C₁₀ linear or branched alkenyl, C₂-C₁₀ linear or branched alkynyl, trifluoromethyl, substituted or unsubstituted aryl, or substituted or unsubstituted heteroaryl; and
wherein R′ is 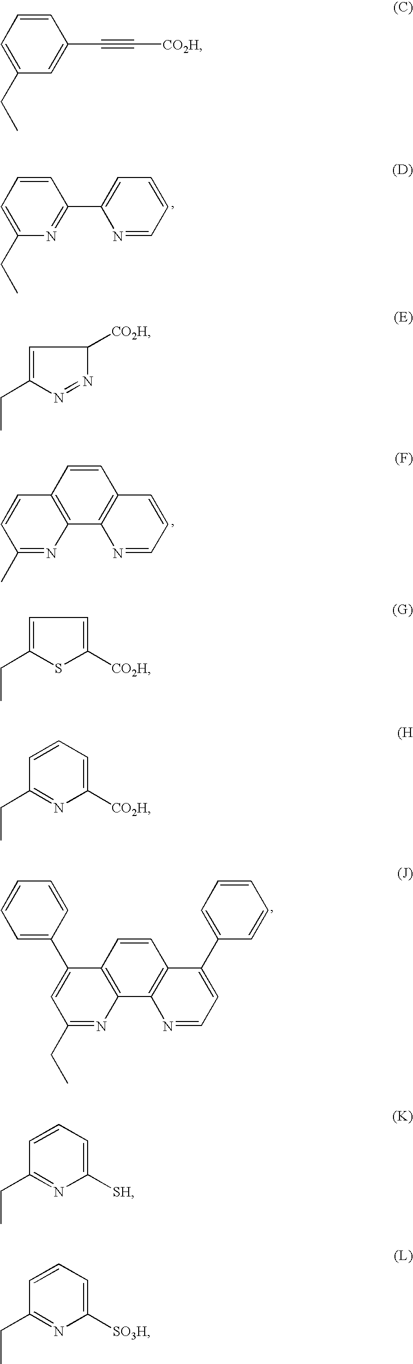 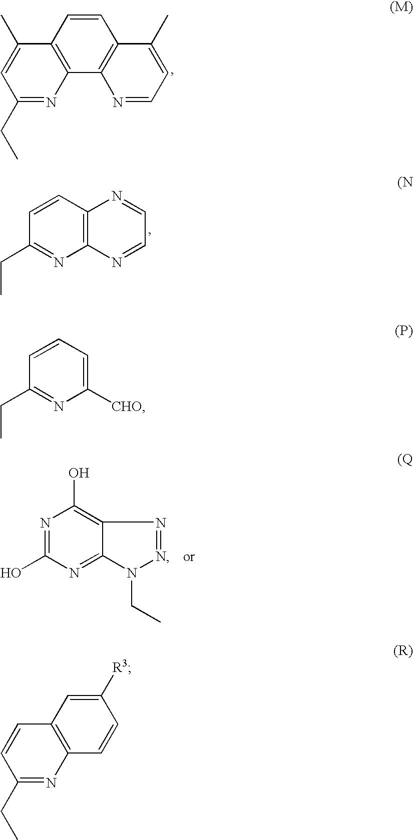
wherein R³ is H or C1-6 alkyl, e.g., methyl, ethyl, propyl or butyl.

In one embodiment in the compound of Formula I or Ia, at least one of R₁ and R₂ is H. In another embodiment of a compound of Formula I or Ia, one of R₁ and R₂ is H and the other is alkyl, e.g., C₁-C₆ alkyl, e.g., methyl, ethyl, n-propyl, isopropyl, cyclopropyl, n-butyl, sec-butyl, tert-butyl, isobutyl, cyclobutyl or pentyl.

In one subembodiment of Formula I or Ia:
p is 0, 1, 2 or 3,
X and Y if present are H or C₁-C₆ alkyl; and
one of R₁ and R₂ is H and the other of R₁ and R₂ is C₁-C₆ alkyl; and
R′ is optionally: 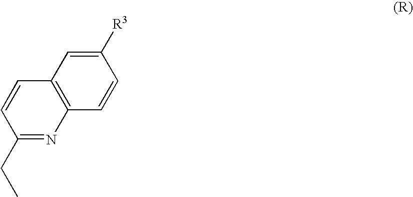
   where R³ is C1-6 alkyl, e.g. methyl, ethyl or propyl.

In another subembodiment of Formula I or Ia:
p is 0, 1, 2 or 3,
X and Y if present are H; and
one of R₁ and R₂ is H and the other of R₁ and R₂ is C₁-C₆ alkyl, e.g., methyl, ethyl, propyl, isopropyl, cyclopropyl, n-butyl, sec-butyl, tert-butyl, isobutyl, cyclobutyl, or pentyl; and
R′ is optionally: 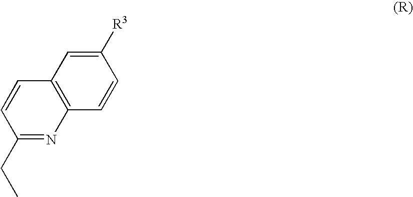
wherein R³ is C1-6 alkyl, e.g., methyl, ethyl or propyl.

In another subembodiment of Formula I or Ia:
p is 0, 1, 2 or 3,
X and Y if present are H; and
one of R₁ and R₂ is H and the other of R₁ and R₂ is C₂-C₆ alkyl, e.g., ethyl, propyl, isopropyl, cyclopropyl, n-butyl, sec-butyl, tert-butyl, isobutyl, cyclobutyl, or pentyl; and
R′ is optionally: 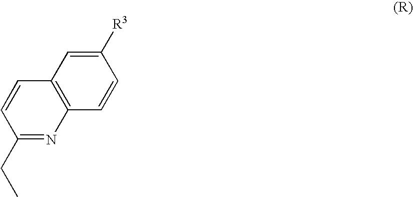
wherein R³ is C1-6 alkyl, e.g., methyl, ethyl or propyl.

In another subembodiment of 1a:
one of R₁ and R₂ is H and the other of R₁ and R₂ is C₁-C₆ alkyl, e.g., methyl, ethyl, propyl, isopropyl, cyclopropyl, n-butyl, sec-butyl, tert-butyl, isobutyl, cyclobutyl, or pentyl; and
R′ is optionally: 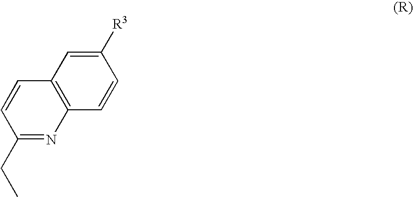
wherein R³ is C1-6 alkyl, e.g., methyl, ethyl or propyl.

In another subembodiment of Formula Ia:
one of R₁ and R₂ is H and the other of R₁ and R₂ is C₂-C₆ alkyl, e.g., ethyl, propyl, isopropyl, cyclopropyl, n-butyl, sec-butyl, tert-butyl, isobutyl, cyclobutyl, or pentyl; and
R′ is optionally: 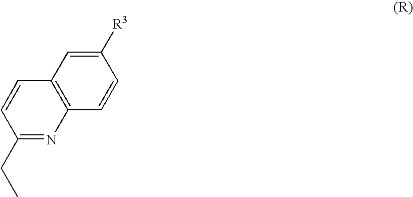
wherein R³ is C1-6 alkyl, e.g., methyl, ethyl or propyl.

In another embodiment, the compound is of Formula II: 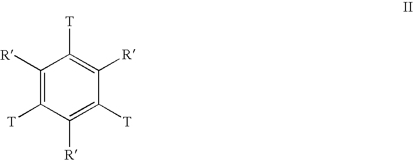
wherein each T is independently 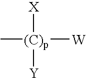
wherein p is 0, 1, 2, 3, 4, 5 or 6;
wherein each X and Y if present are independently H, OH, C₁-C₆ alkyl, substituted or unsubstituted aryl, or unsubstituted or substituted heterocycle;
wherein each W independently is 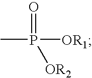
   wherein each R₁ and R₂ are independently H, C₁-C₁₀ linear or branched alkyl, C₂-C₁₀ linear or branched alkenyl, C₂-C₁₀ linear or branched alkynyl, trifluoromethyl, substituted or unsubstituted aryl, or substituted or unsubstituted heteroaryl;
wherein each R′ is independently H, or C₁-6 alkyl, which is optionally substituted.

In one embodiment in the compound of Formula II, at least one of R₁ and R₂ is H. In another embodiment of a compound of Formula II, one of R₁ and R₂ is H and the other is alkyl, e.g., C1-6 alkyl, or is, e.g. methyl, ethyl, n-propyl, isopropyl, cyclopropyl, n-butyl, sec-butyl, tert-butyl, isobutyl, cyclobutyl or pentyl.

In one subembodiment of Formula II:
p is 1, 2 or 3;
X and Y are H or C1-6 alkyl;
one of R₁ and R₂ is H and the other is C1-6 alkyl; and
R′ is H or C1-6 alkyl.

In another subembodiment of Formula II:
p is 1, 2 or 3;
X and Y are H;
one of R₁ and R₂ is H and the other is C1-6 alkyl, e.g., methyl, ethyl, propyl, n-butyl, sec-butyl, tert-butyl, isobutyl or cyclobutyl; and
R′ is H or C1-6 alkyl, e.g., methyl, ethyl or propyl.

In another subembodiment of Formula II:
p is 1 or 2:
X and Y are H;
one of R₁ and R₂ is H, and the other is C₂-C₆ alkyl, e.g., ethyl, propyl, n-butyl, sec-butyl, tert-butyl, isobutyl, cyclobutyl or pentyl; and
R′ is H.

In another subembodiment of Formula II:
p is 1 or 2;
X and Y are H;
one of R₁ and R₂ is H, and the other is C₃-C₆ alkyl, e.g., propyl, n-butyl, sec-butyl, tert-butyl, isobutyl, cyclobutyl or pentyl; and
R₁ is C₁-C₆ alkyl, e.g. methyl, ethyl or propyl.

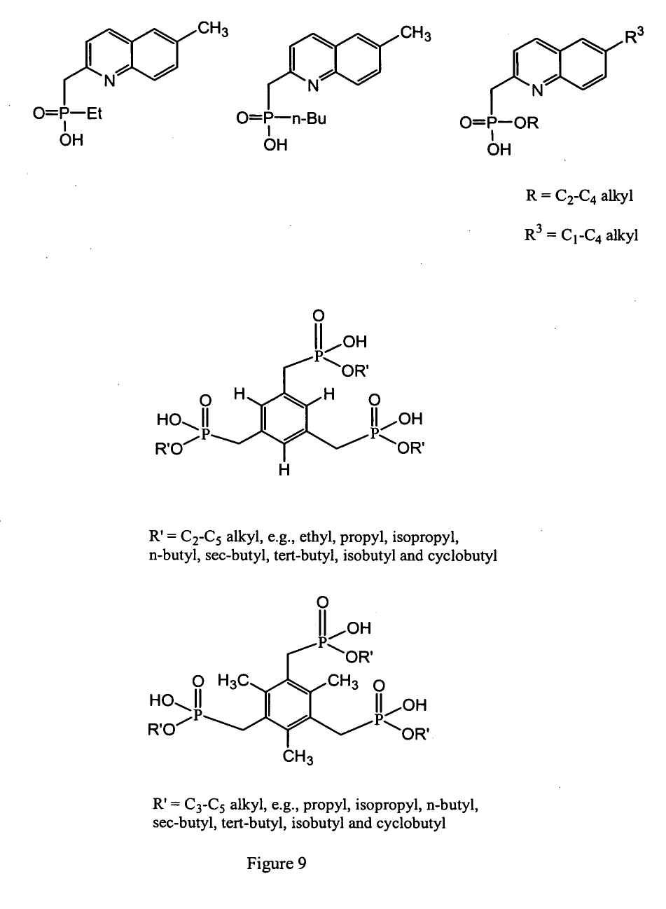

Exemplary compounds of Formula I, Ia and II are shown in the Figures, including FIG. 9.

In another embodiment, the compound is a chelant which is a tetraazamacrocyclic compound as described in PCT WO 03/035114, published May 1, 2003; PCT WO 03/035655, published May 1, 2003, or US 2003/0099598, published May 29, 2003, the disclosures of which are incorporated herein by reference, e.g., a compound of Formula IV (see, e.g., US Appl. Publ. No. 3003/0133872, published Jul. 17, 2003, the disclosure of which is incorporated herein): 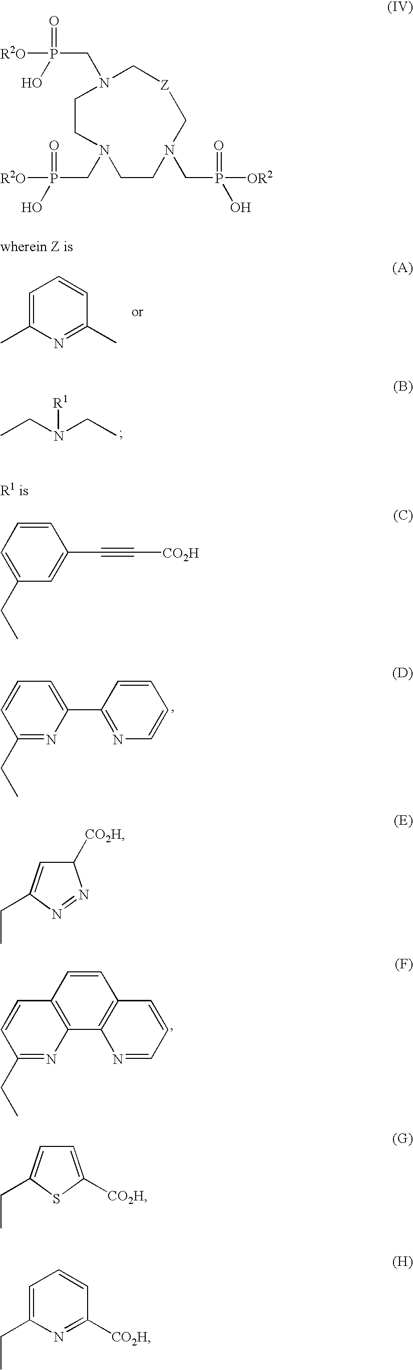 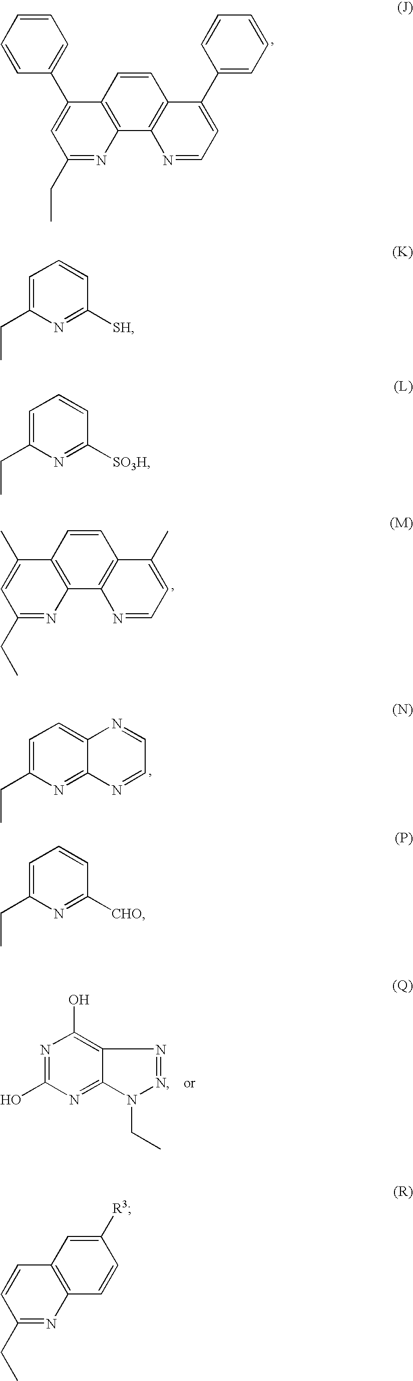
R² is methyl, ethyl, propyl, butyl or H; and
R³ is F, C₁-C₄ alkyl, O(C₁-C₄ alkyl), or Cl, or a salt thereof.

In one particular embodiment Z is B, R² is n-butyl and R¹ is R in which R³ is a methyl, which is the QCTME chelant:

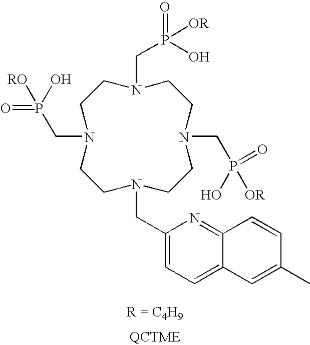

The compounds, including the chelants of Formula IV and QCTME can be used in the diagnostic methods and therapeutic methods disclosed herein.

In another embodiment, the compound is a compound of Formula IVa: 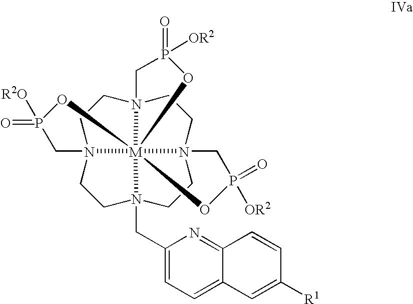
wherein R2 is H or C1-6 alkyl, e.g, methyl, ethyl, propyl, n-butyl, sec-butyl, tert-butyl, isobutyl or cyclobutyl, and R₁ is-C1-6 alkyl, e.g., methyl, ethyl or propyl, and where M if present is a metal ion.

In one embodiment, the compound is a chelant which is a polyaminophosphonic acid metal complex of Formula (V): 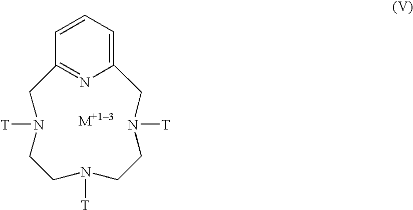
wherein each T is independently 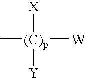
wherein p is 0, 1, 2, 3, 4, 5 or 6; and
wherein each X and Y if present are independently H, OH, C₁-C₆ alkyl, substituted or unsubstituted aryl, or unsubstituted or substituted heterocycle;
wherein W is 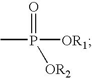
wherein each R₁ and R₂ are independently H, C₂-C₁₀ linear or branched alkyl, C₂-C₁₀ linear or branched alkenyl, C₂-C₁₀ linear or branched alkynyl, trifluoromethyl, substituted or unsubstituted aryl, or substituted or unsubstituted heteroaryl; and
wherein M is a metal cation having a valency of at least +1.

In one embodiment, T is CH₂PO(OR₁)OR₂, wherein R₁ is H, R₂ is a C₂-C₆ alkyl, and M is Th (TbPCTMB).

The term “alkyl”, as used herein and unless specified otherwise, includes a saturated, straight, branched, or cyclic, primary, secondary or tertiary hydrocarbon radical of for example C₁ to C₁₀, and specifically includes methyl, ethyl, propyl, isopropyl, cyclopropyl, butyl, isobutyl, t-butyl, pentyl, cyclopentyl, isopentyl, neopentyl, hexyl, isohexyl, cyclohexyl, cyclohexylmethyl, methylpentyl and dimethylbutyl.

Throughout the specification, when a range is specified, this is meant to independently include every member of the range. For example, the range “C₁-C₆ alkyl” independently includes C₁, C₂, C₃, C₄, C₅, and C₆ alkyl.

The term “alkenyl” as used herein, unless otherwise specified, includes an acyclic, straight, branched, or cyclic, primary, secondary, or tertiary hydrocarbon radical, including those containing from 2 to 10 carbon atoms containing at least one carbon-carbon double bond. Examples of such radicals include ethylene, methylethylene, and isopropylidene.

The term “alkynyl” as used herein, unless otherwise specified, includes an unsaturated, acyclic hydrocarbon radical, linear or branched, in so much as it contains one or more triple bonds, including such radicals containing about 2 to 10 carbon atoms. Examples of alkynyl radicals include ethynyl, propynyl, hydroxypropynyl, butyn-1-yl, butyn-2-yl, pentyn-1-yl, pentyn-2-yl and the like.

The term “halo” as used herein, unless otherwise specified, includes chloro, bromo, iodo, and fluoro.

The term “aryl”, as used herein, unless specified otherwise, includes phenyl, biphenyl and naphthyl.

The term “heterocycle” as used herein, unless otherwise specified, includes nonaromatic cyclic groups that may be partially (e.g., contains at least one double bond) or fully saturated and wherein there is at least one heteroatom, such as oxygen, sulfur, nitrogen, or phosphorus in the ring. Similarly, the term heteroaryl or heteroaromatic, as used herein, refers to an aromatic that includes at least one sulfur, oxygen, nitrogen or phosphorus in the aromatic ring. Nonlimiting examples of heterocylics and heteroaromatics include pyrrolidinyl, tetrahydrofaryl, piperazinyl, piperidinyl, morpholino, imidazolyl, pyrolinyl, pyrazolinyl, and indolinyl.

Where a group is indicated as being “substituted”, the group may be substituted in one or more positions for example with halo, hydroxyl, amino, nitro, azido, cyano, sulfonic acid, sulfate, alkyl, alkenyl, alkynyl, heteroaryl, heterocyclic, carbohydrate, amino acid, acyl, carboxylic ester, carboxylic acid, amide, etc., any or all of which may be unprotected or further protected as necessary, as known to those skilled in the art and as taught, for example, in Greene et al., Protective Groups in Organic Synthesis, John Wiley and Sons, 2^{nd} Edition (1991).

III. Chelates

The compounds can be used by themselves or comprise one or more cations, e.g., lanthanide cations, and radionuclides and thus be in the form of a chelate. Some such chelates may form after administration by complexation of the compound with a cation. As used herein, in a complex of a chelate and a metal ion, the term “metal ion” includes, e.g., a lanthanide cation or a radionuclide. The cation, e.g. metal or radionuclide, chosen will depend upon the most appropriate cation, metal ion or isotope for therapeutic or diagnostic purposes, and will depend upon a number of factors including cell or tissue (e.g., tumor) uptake and retention, blood clearance, rate of radiation delivery, half-life, specific activity of the radionuclide, and degree of side-effects associated with use.

As further characterized herein, useful metal cations include those that exhibit a therapeutic effect per se (e.g., therapeutic radioisotopes); those that (e.g., by radioactive emission or by fluorescence in the chelate) permit detection of the chelate and thus permit, e.g., surgical therapy; and those that can, once delivered to the abnormal cell (such as a diseased or perturbed cell), be activated by the application of one or more stimuli in order to exert a therapeutic effect. Diagnostic metal cations include those giving off a detectable signal. The signal can be but is not limited to gamma emission (nuclear medicine applications such as scintigraphy, SPECT, and PET), visible light (optical applications), radiofrequency (MR imaging). In addition, metal ions of this invention include contrast agent applications for CT and X-ray.

Cations

Diagnostically and therapeutically useful cations, atoms, and ions as described herein include those that (e.g., by radioactive emission, or by fluorescence by the metal cation-complexed chelate) in one embodiment permit detection of the chelate and thus help a practitioner to diagnose the presence of an abnormal cell into which the chelate has specifically been uptaken. In one embodiment, the diagnostically useful cation, atom, or ion will be useful in any one or more of the following for the general applications, including but not limited to: nuclear magetic resonance (NMR) or magnetic resonance imaging (MRI); X-ray or X-ray computed tomography; positon emission tomography (PET); gamma scintigraphy; Computed Tomography (CT) and Single Photon Emission Computed Tomography (SPECT); and optical imaging.

For use with complexes of the present invention in Positron Emission Tomography (PET) applications, the metal, atom or ion should preferably be a cation including but not limited to ⁶²Cu, ⁷⁴As, ⁵⁵Co, ⁶¹Cu, ⁶⁴Cu, ⁶⁸Ge, ⁵²Mn, ⁸⁶Y, ⁸⁷Y, or ⁸²Rb. Alternatively and equally acceptable, the atom or ion suitable for use in PET applications can be a non-chelated, covalently attached atom or element, including but not limited to ¹⁸F, ¹²⁴I, ¹¹C, ¹³N, ¹⁵O, or ⁷⁵Br.

In the instance of gamma scintigraphy and/or Single Photon Emission Computed Tomography (SPECT) applications of the chelates, complexes, and of the present invention, that atom, cation, or metal atom or ion should preferably be a cation such as ^{99m}Tc, ¹¹¹In, or ⁶⁷Ga. Alternatively and equally acceptable, ECT, SPECT and gamma scintigraphy applications can be achieved using a non-chelated, covalently attached atom or ion including but not limited to ¹²¹I, or ¹³¹I.

When complexes are used in optical imaging applications, in one embodiment, a chelant be complexed with rare earth cations. Optionally, the complex can be between any of the chelants and a divalent or higher valency lanthanide, including Tb, Eu, Dy, and La. In one embodiment, the cation is other than Tb, Eu, Dy, and La. In another embodiment, the cation is selected from La, Ce, Pr, Nd, Pm, Sm, Eu, Gd, Tb, Dy, Ho, Er, Tm, Yb or Lu. In another embodiment, the cation is selected from Bi, Ac, Th, Pa, or U.

When complexes are used in therapeutic applications, a chelant can be complexed with a cation such as a rare earth cation. Optionally, the complex can be between any of the chelants and a divalent or higher valency lanthanide, including Tb, Eu, Dy, and La. In one embodiment, the cation is other than Tb, Eu, Dy, and La. In another embodiment, the cation is selected from La, Ce, Pr, Nd, Pm, Sm, Eu, Gd, Tb, Dy, Ho, Er, Tm, Yb or Lu. In another embodiment, the cation is selected from Bi, Ac, Th, Pa, or U. Moreover, chelants can be complexed with any other of the cations disclosed herein for therapeutic applications, including the treatment of cancer conditions, including tumor treatment.

Radionuclides

When radionuclides are used, the radionuclides have a half-life sufficiently long so as to allow for localization and delivery of the complex or conjugate in the target cell or tissue while still retaining sufficient radioactivity to achieve the desired goal (diagnostic or therapeutic). Generally, when radionuclides are used, it is preferred to use a radionuclide-ligand complex that results in rapid biolocalization of the radionuclide in the cell or tissue so as to achieve rapid onset of irradiation. In preferred embodiments, radionuclides having sufficient alpha- or beta-energy so as to be therapeutically useful are used. Radionuclides utilized in the methods of the present invention exhibit for example a maximum beta energy of from greater than about 0.1 MeV to greater than about 2 MeV.

As used herein, the term “radionuclide” includes an unstable isotope of an element that decays or disintegrates, spontaneously emitting radiation. In general, particulate radioactive decay (betas, electrons, alphas) are useful for therapy and electromagnetic radiation (gammas) are useful for diagnostic applications.

Radionuclides which are useful in the methods and compositions of the present invention include, but are not limited to, Arsenic-77 (⁷⁷As), Rhodium-105 (¹⁰⁵Rh), Lutetium-177 (¹⁷⁷Lu), Cadmium-115 (¹¹⁵Cd), Antimony-122 (¹²²Sb), Promethium-149 (¹⁴⁹Pr), Osmium-193 (¹⁹³Os), Gold-198 (¹⁹⁸Au), Tin-117m (^{117m}Sn), Strontium-89 (⁸⁹Sr), Indium-115m (^{115m}In), Dysprosium-165 (¹⁶⁵Dy), Lanthanum-140 (¹⁴⁰La), Ytterbium-175 (¹⁷⁵Yb), Scandium-47 (⁴⁷Sc); preferably Samarium-153 (¹⁵³Sm), Yttrium-90 (⁹⁰Y), Gadolinium-159 (¹⁵⁹Gd), Rhenium-186 (⁸⁶Re), Rhenium-188 (188 Re), and Holmium-166 (¹⁶⁶Ho). Especially preferred is ¹⁶⁶Ho, which emits high-energy beta particles and gamma radiation (80 KeV, 6.0%) useful for imaging and exhibits a half-life of 26.8 hr. In other embodiments, alpha emitters such as Actinium-225 (²²⁵Ac), Bismuth-212 (²¹²Bi) and Bismuth-213 (²¹³Bi) can be utilized.

In one embodiment, the therapeutically useful metal will be selected from the cations ¹⁶⁶Ho, ⁹⁰Y, ¹⁵⁹Gd, ¹⁷⁷Lu, ¹¹¹In, ^{115m}In, ¹⁷⁵Yb, ⁴⁷SC, ²²⁵Ac, ²¹²Bi, ²¹³Bi, ¹⁴⁹Pm, ¹⁴⁰La, ¹⁴²Pr, ¹⁸⁶Re, or ¹⁸⁸Re.

The radionuclides suitable for use herein, such as those described above, can be obtained using procedures well known in the art. Typically, the desired radionuclide can be prepared by bombarding an appropriate target, such as a metal, metal oxide, or salt with neutrons. Another method of obtaining radionuclides is by bombarding nuclides with particles in a linear accelerator or cyclotron. Yet another way of obtaining radionuclides is to isolate them from fission product mixtures. The present invention is not limited to a particular method of obtaining radionuclides. Any suitable method that results in the generation of the desired radionuclide may be utilized.

As used herein, the term “chelant” includes a phosphonic acid half ester-containing molecule.

As used herein, the term “chelate” includes a complex of a chelant with a mono-, di-, tri-, tetra-, penta-, or hexa-valent cation. The cation may be a metal, e.g., a lanthanide or transition metal cation. The chelate may be a simple complex with the cation, involving only non-ionic-bond, non-covalent attractions, or it may be a complex involving both ionic bonds and other non-covalent attractions. In the latter case, the chelant may become ionized by reaction with the cation and/or with a solvent, e.g., water. For example, an oxo-acid-type group of the (neutral) chelant, e.g., a phosphonate group or phosphonate ester group, may lose a hydrogen from a hydroxyl thereof, and the resulting oxide moiety might then participate in ionic bonding with the metal ion.

Formation of Chelates

The metal or cation and ligand may be combined under any conditions which allow the two to form a complex. Generally, mixing in water at a controlled pH (the choice of pH is dependent upon the choice of metal) is all that is required. In one embodiment, the desired amount of ligand is placed in a vial and dissolved by addition of water. The appropriate amount of the cation or metal is then added to the ligand solution. The pH of the resulting solution is then adjusted to the appropriate level (e.g., 7-8). Additionally, the complex may be a mixture of the different metals or cations.

In formation of the chelate, the complex can be formed or used in the presence of an excess of ligand. The ligand to metal ratio (L:M) of the ligand to radionuclide or metal in one embodiment is at least 50:1. The upper limit of L:M depends on the toxicity of the ligand or the nature of the cation or metal ion. The exemplary range for the L:M ratio is from 50:1 to 600:1, preferably from 100:1 to 500:1, especially 250:1 to 300:1.

IV. Targeting Properties of the Compounds

The targeting ability of chelants, chelates, and other molecules and complexes disclosed herein, for abnormal and disease-state cells can be attributable to a number of properties without being limited to any theory. Advantageous properties, including dimerization, can also unexpectedly be exhibited. Useful properties of the compounds include the following parameters: phosphonate positioning/orientation; logP octanol/water partitioning; molecular morphology (including, e.g., ionic charge, molecular weight, molecular shape and dimensions); and suitability for in vivo use (stability, toxicity etc.).

1) Phosphonate Structure

Compounds of the present invention are found to exhibit cell membrane permeability and protein binding properties. One chemical feature that is in one embodiment common to these molecules is the presence of one or more C₁-C₁₆ aliphatic phosphonate ester moieties, preferably mono-aliphatic phosphonate esters, more preferably monoalkyphosphonate ester moieties. While not wishing to be bound by theory, it is possible that these groups serve the following purposes in some embodiments: 1) creation of a formal bond to a metal ion generating a stable and inert complex; and/or 2) generation of an organized hydrogen bonding network with amino acid segments of peptides and proteins. Phosphinoxide groups are strong hydrogen bond acceptors and weak Bronsted bases. The high dipole moment of these functionalities make them well suited for molecular recognition of cations. However, due to the presence of phosphinoxide isomers and the fact that phosphorus usually functions as a sterogenic center, synthetic cation receptors containing multiple P═O groups have rarely been reported. This problem is eliminated herein by the presence, in solution, of achiral charged phosphonate anions, which exhibit even more pronounced polarization; additionally, these groups offer the further advantage of being fully dissociated at physiological pH due to their relatively low pKₐ values (1.8 versus 4.8 for carboxylates). In addition, the phosphonate ester functionality can serve as a handle for introducing lateral recognition of a substrate binding region.

2) In Vivo Compatibility

In one embodiment, the compounds are suitable for in vivo administration. In one embodiment, the chelant binds to a cation with sufficient avidity to prevent release of the cation into soft tissue, which could result in toxicity side-effects. In some embodiments, chelates are derived from chelants that produce thermodynamically stable chelates that do not dissociate in such a manner, under biological conditions.

3) Other Properties

In some instances, the compounds can exhibit enhanced uptake into targeted cells. Formulations of the targeting chelates can be prepared for topical or systemic delivery. In the case of topically applied chelate formulations (for example application to epithelial tissue of the colon, oral cavity and GI tract) optimal uptake by the target tissue is, in one embodiment less than 45 minutes, or less than 10 minutes. For drug formulation designed for intravenous injection, maximum uptake time will be influenced by tissue/tumor permeability, the number of receptor sites, etc. Ideally, for diagnostic imaging applications uptake time will be optimal within two hours of injection. For therapeutic applications optimal uptake is, e.g., in less that 24 hours, or within 12 hours.

In addition, one further advantageous property of the compounds is that, after uptake into abnormal and disease-state cells, they can potentially exhibit a greater cellular residence time than, e.g., some anti-cancer drugs. It is frequently observed that cancer cells respond to uptake of an anti-cancer agent by effecting one or more export pathway that efficiently secretes or otherwise transports the pharmaceutical agent from the cell, thereby decreasing its effectiveness for the intended purpose. For example, acquired anti-cancer drug resistance has been observed toward both anti-cancer antibiotics, such as doxorubicin, and anti-cancer chelates, such as cisplatin. The transporter pathways involved in drug export include the multiple drug resistance protein family members and the metal export pump proteins. M. Yoshida et al., Int'l J. Cancer 94(3):432-37 (Nov. 1, 2001) ; K. Katano et al., Molec. Pharmacol. 64(2):466-73 (August 2003). These pathways are typically synthesized or activated within a few minutes or up to about 2 hours after exposure to the agent, with increasing loss of the agent from the cell thereafter. In some embodiments, the compounds disclosed herein remain in the cell without being exported by such a pathway for at least 8 hours in abnormal diseased cells.

Thus compounds having the phosphonate alkyl half-ester characteristics specified herein can provide increased in cyto residence time to detection/diagnostic and/or treatment/therapeutic agents for use in abnormal and disease-state cells, in one embodiment by selecting as a partner for covalent and/or non-covalent conjugate formation herein, any such agent that is susceptible to secretion or export by an abnormal or disease-state cell or tissue.

In one embodiment, the compounds have a log P of about 0-4. The partition coefficient is known as the ratio of concentration of compound in aqueous phase to the concentration in an immiscible solvent, as the neutral molecule. In practical terms the neutral molecule exists for bases >2 pKa units above the pKa and for acids >2 pKa units below. The log P is calculated as shown below.

Partition Coefficient, P=[Organic]/[Aqueous] Where [ ]=concentration

Log P=log 10 (Partition Coefficient)

Both experimental and theoretical methods have been developed for common organic compounds containing C, H, O, N and S atoms. An ab initio quantum chemistry method, combined with quantum structure activity relationships (QSPR) can be used in general for virtually any compound, provided that accurate quantum chemistry basis fuction is available for every atoms of the molecule. Other methods are also known in the art for measurement and for calculation of log P values. For example, calculations can be performed according to the algorithm of V. N. Viswanadhan et al., J. Chem. Inf. Comput. Sci. 29(3):163-72 (1989), which is available in an automated form as the LOGP CALCULATOR (software plug-in available from ChemAxon, Budapest, Hungary).

V. Preparation of Compounds and Conjugates

Methods for the synthesis of biologically compatible chelants and chelates and conjugates thereof are well known in the art, and have been particularly developed for construction of lanthanide and other cation chelates, which are widely employed for diagnostic and therapeutic applications. Thus, for example, see “Gadolinium (III) Chelates as MRI Contrast Agents: Structure, Dynamics, and Applications,” Chemical Reviews 99(9):2293-2353 (1999); and “Bifunctional Chelators for Lanthanide Radiopharmaceuticals,” Bionconjugate Chemistry 12:7-34, (2001). Such procedures are exemplary of useful synthesis methods useful for preparation of chelants and chelates in some embodiments. Once the chelant is formed, it can be complexed if desired, for example, for a polyazamacrocyclic chelant, with a divalent or greater cation to form a chelate. In the case of lanthanide cations, a typical lanthanide complexation procedure involves: combining an amino-phosphonate ligand with a. trivalent lanthanide metal salt or oxide initially under aqueous acidic conditions, the mixture having an initial pH below pH6, typically within the range of pH 2 to pH 6; titrating the resulting solution or suspension reaction mixture with a base to maintain the reaction mixture within a range of about pH4 to pH6 in order to facilitate complexation by counteracting the generation of excess protons by the complexation reaction, until pH fluctuations subside. The aqueous reaction mixture may optionally contain a buffering agent, such as ammonium acetate, or ascorbic acid as described in U.S. Pat. No. 6,713,042.

After the complexation reaction is complete, if there is a metal ion, the pH may be brought to a desired level, preferably by first gradually raising the pH to about pH 8 and then modifying the pH to a level desired for administration of the resulting chelate. In the case of compositions for administration, a desired pH level may be, e.g., a pH within the range of about pH 2 to about pH 10, more preferably a pH from about pH 4 to about pH 9.

The complex formed by the complexation reaction is a thermodynamically stable chelate structure, i.e. stable to the disassociation of a chelated +3 metal ion from the ligand under biological conditions, as well as under a wide range of pH conditions. (Formation of transition metal and non-metal cations of +1, +2, +4, +5, +6, and +7 charge, if present, may be similarly performed.) Afterwards, the resulting chelate may be, e.g., frozen, dried, or lyophilized and/or may be combined with other desired component(s) to produce a formulation for use or administration.

Chelants can be made as described in PCT WO 93/11802, the disclosure of which is hereby incorporated by reference. In addition, similar polycyclic (tri-and tetra-cyclic) chelants are described in U.S. Pat. No. 5,385,893 and PCT Publication WO 94/26726, the disclosures of which are hereby incorporated by reference. Chelants can be made as described in U.S. Pat. No. 5,462,725 and PCT WO 94/26275, the disclosures of which are hereby incorporated by reference.

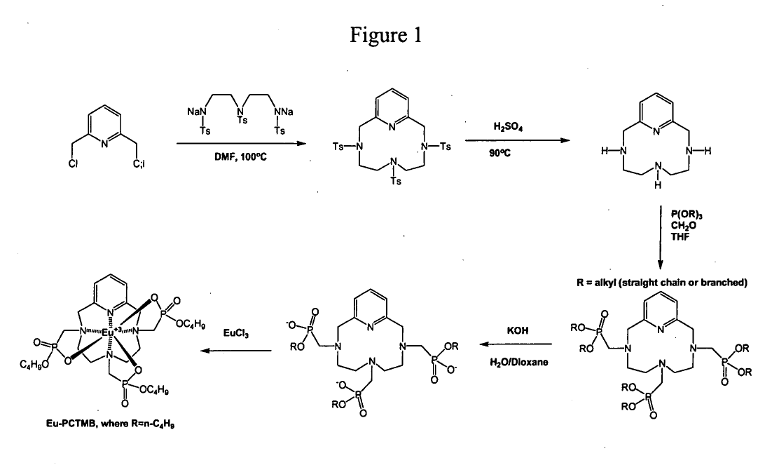

Scheme 1 (see FIG. 1) illustrates the synthesis for preparing a chelant, followed by complexation with a cation to form a chelant. In this illustration, tosylated diethylene tetramine sodium salt is obtained by tosylation and conversion of diethylene tetramine to the sodium salt in a separate step. 2,6-Bis(chloromethyl)pyridine (achieved by treating 2,6-bis(hydroxymethyl)pyridine with thionyl chloride) is then converted to a tosylated macrocycle via the reaction with the tosylated diethylene tetraamine sodium salt in dimethyl formamide (DMF). Deprotection of the amines is then accomplished by heating to about 90° C. in sulfuric acid. The N-alkyl phosphonate esters are then synthesized by reacting the secondary amines of the macrocycle with a trialkyl phosphite and paraformaldehyde in tetrahydrofuran (THF). The resulting phosphonate ester is then selectively hydrolyzed under basic conditions to give the monoalkyl phosphonate, which forms stable chelates with numerous metals having at least a +2 charge, such as those in the lanthanide series, by contacting the phosphonate ester with a metal chloride (such as EuCl₃ or TbCl₃).

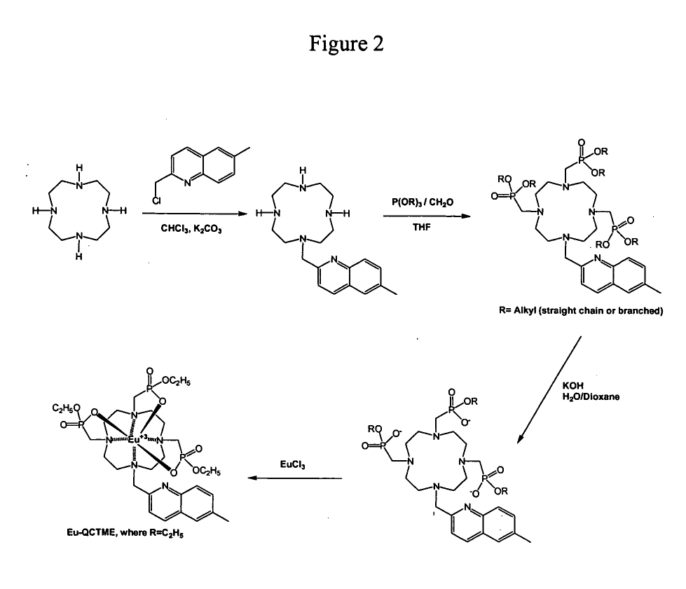

Scheme 2 (see FIG. 2) illustrates the synthesis of a chelate that contains a 12-membered tetraazamacrocycle possessing a substituted quinoline pendant moiety attached at one of the macrocyclic secondary nitrogen positions. 2-Chloromethyl-6-methyl quinoline is first prepared by reacting 4-methyl aniline with butyraldehyde in 6M HCl to form 2-methyl-6-methyl quinoline, according to the general procedure previously described by Leir (J. Org. Chem., Vol. 42: pp. 911-913 (1977)). This quinoline compound is then reacted with 3-CPBA (3-chloro-peroxybenzoic acid) to yield 2-methyl-6-methylquinolone N-oxide. Deprotection with tosyl chloride (or a similar deprotection agent) and simultaneous methyl-chlorination using the method of Butera, et al. (J. Med. Chem., Vol. 34: pp.3212-3228 (1991)) produces 2-chloromethyl-6-methyl-quinoline. Covalent attachment of the quinoline moiety is then achieved by reacting 1,4,7,10-tetraazacyclododecane with 2-chloromethyl-6-methyl quinoline in an aprotic solvent such as CHCl₃, CH₃CN, or DMF in the presence of a base (such as K₂CO₃, Na₂CO₃, or CsCO₃) at room temperature to form 1-[2-(7-methyl)methylene-quinolinyl]-1,4,7,10-tetraazacyclododecane. The N-alkyl phosphonate esters can be prepared by reacting the secondary amines of the macrocycle with a trialkyl phosphate (such as tributyl phosphite or triethyl phosphate) and paraformaldehyde in a solvent such as tetrahydrofuran (THF). The resulting phosphonate ester can the be hydrolyzed under basic conditions (KOH, H₂O/dioxane) to give the 1-[2-(6-methyl)methylenequinolinyl]-1,4,7,10-tris(methylene-phosphonic acid n-alkyl ester)-1,4,7,10-tetraazacyclododecane product. Alternatively, the phosphonate ester can be hydrolyzed under acidic conditions to produce the phosphonic acid derivative. Conversion to the desired complex can then be conducted as outlined generally in Scheme 1 by reacting with the appropriate metal ion (such as EuCl₃).

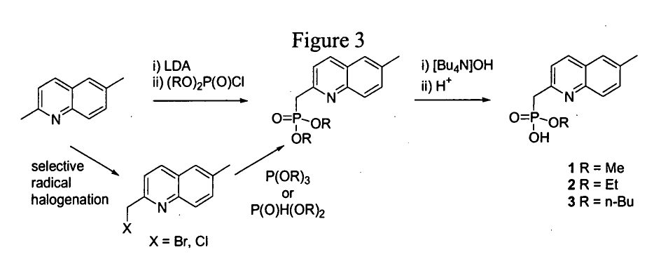

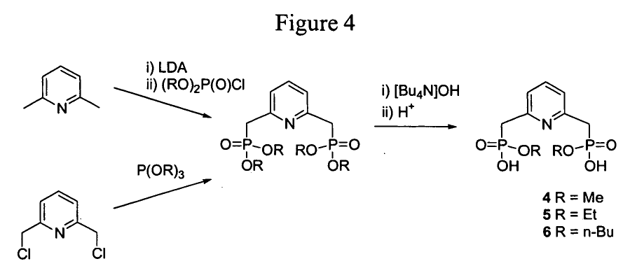

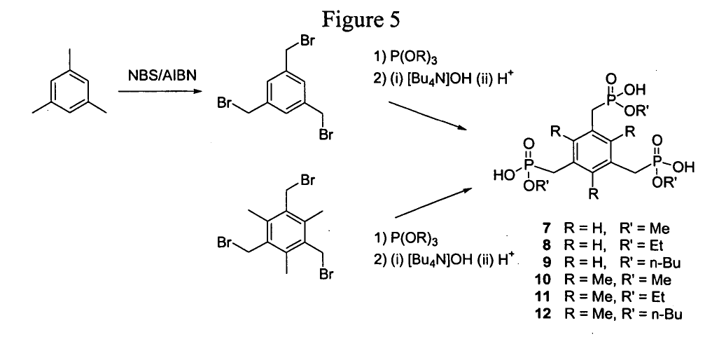

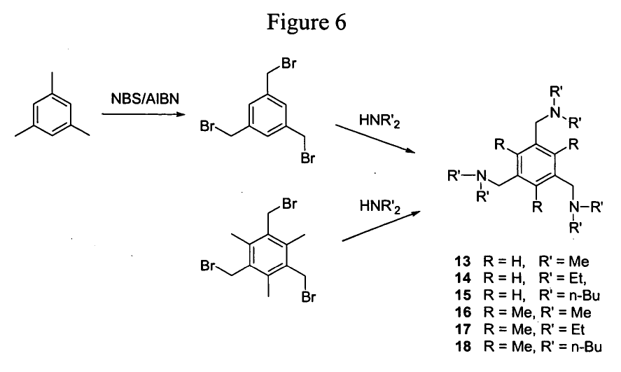

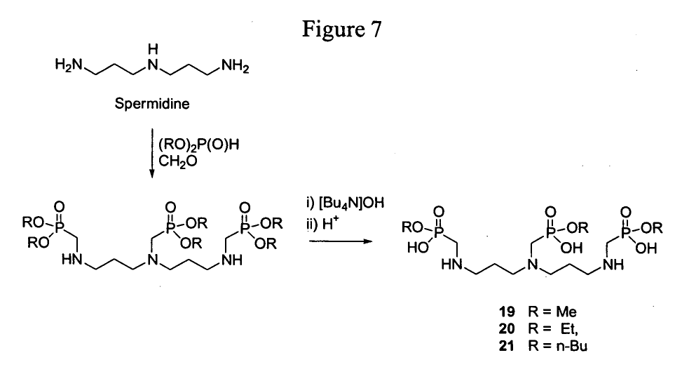

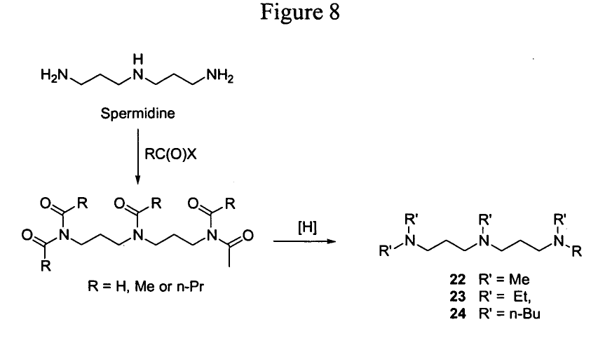

FIGS. 3-8 also show exemplary methods of synthesis of compounds of Formula I, Ia or II. The syntheses shown in FIGS. 3-8 and the Examples can be readily modified to permit the preparation of other phosphonic acid monoalkylesters as described herein, by the selection of the appropriate starting materials and reagents using knowledge available in the art and the techniques described herein. For example, the appropriate dialkyl chlorophosphate or trialkylphosphite reagents can be selected to obtain the desired product.

In one embodiment, a therapeutic agent is covalently linked to or non-covalently associated with the compounds disclosed herein using chemistry techniques available in the art. Methods are available in the art for using linkers to attach biological agents to compounds as described for example, in U.S. Pat. No. 5,435,990 and U.S. Pat. No. 5,652,361, the disclosures of which are incorporated herein by reference. Therapeutic agents that can be covalently attached or non-covalently associated with the compounds disclosed herein include alkylating agents, such as nitrogen mustards; nitrosureas; folic acid analogs, such as methotrexate and trimetrexate; pyrimidine analogs, such as 5-fluorouracil, fluorodeoxyuridine, gemcitabin, cytosine arabinoside, 5-azacytidine, and 2,2′-difluorodeoxycytidine; purine analogs, such as 6-mercaptopurine, 6-thioguanine , and azathioprine; natural products, including antimitotic drugs such as paclitaxel (Taxol®); antibiotics, such as actimomycin D, daunomycin (rubidomycin), doxorubicin (adriamycin) and other anthracycline analogs, mitoxantrone, idarubicin, bleomycins, plicamycin (mithramycin), mitomycinC, dactinomycin, and tobramycin; platinum coordination complexes such as cisplatin and carboplatin; anthracenedione; mitoxantrone; substituted ureas, such as hydroxyurea; cytokines, such as interferon alpha, beta, and gamma and Interleukin 2 (IL-2); and dihydrofolate reductase.

VII. Formulations and Administration

Hosts, including mammals and particularly humans, suffering from a disorder, can be treated by administering to the host an effective amount of a compound or conjugate as described herein, or a pharmaceutically acceptable salt thereof, optionally in combination with a pharmaceutically acceptable carrier or diluent. The active materials can be administered by any appropriate route, for example, orally, parenterally, intravenously, intradermally, intramuscularly, subcutaneously, sublingually, transdermally, bronchially, pharyngolaryngeal, intranasally, topically such as by a cream or ointment, rectally, intraarticular, intracistemally, intrathecally, intravaginally, intraperitoneally, intraocularly, by inhalation, bucally or as an oral or nasal spray.

When used for an in vivo diagnostic or therapeutic purpose, the compound, or composition containing the same, may be administered to any animal, preferably a vertebrate animal (e.g., a bird, fish, or reptile), more preferably a mammal; or to a human subject. Exemplary mammal subjects include, e.g., dogs, cats, mice, rats, hamsters, guinea pigs, horses, cattle, sheep, goats, monkeys, apes, and the like.

A compound or composition may be applied or administered to a subject in a variety of modes, whether at the location of a suspected or otherwise indicated abnormal or disease-state cell or tissue, or systemically (e.g., peripherally). Administration may be performed by any convenient route, whether systemically/peripherally or at the site of desired action, including but not limited to, topical, oral (e.g. by ingestion); parenteral, for example, by injection, performed in any desired mode, e.g., intraarterial, intraarticular, intracardiac, intrathecal, intraspinal, intratracheal, intravenous, subarachnoid, and so forth.

An exemplary mode of administration is topical application, including any one of, e.g., non-invasive topical application such as sublingual, buccal, intranasal, ocular, dermal, or transdermal, rectal, or vaginal application, or pulmonary application as through insufflating or inhaling through the nose or mouth a, e.g., powder or aerosol; and invasive topical application (whether applied to a site accessed by surgical scission or by catheter or needle) such as application to the peritoneum, reproductive tract, stomach, colon, and so forth. In a topical mode, any useful application format may be employed to the selected tissues or cell surface as, e.g., washing, lavage, swabbing, painting, spraying, and so forth.

Another mode of application is by topical administration over a human or animal tissue that has been removed from the organism. This is referred to as ex vivo administration.

For detection and diagnosis purposes, administration may be advantageously followed by endoscopy (in any format, including, e.g., capsule endoscopy) or radiometry to observe a, e.g., fluorescent or radioactive chelant, or chelate, or, where the chelant, or chelate, is non-invasively detectable by a remote method, administration may be followed by any non-invasive detection technique (such as MRI, X-ray, PET, and so forth); or where the cells or tissues targeted by the compound are surface-accessible (e.g., in the oral cavity), administration may be followed by any surface-accessible detection technique (e.g., non-endoscopic fluoroscopy, or radiometry).

In one embodiment properties of the compounds disclosed herein, in particular the very fast, selective cell uptake kinetics and their long cellular and/or intracellular residence time, make them particularly advantageous for use in conjunction with surgical procedures for excision or ablation of abnormal cells and tissues. In some embodiments, topical application benefits greatly, since an accessible or accessed tissue surface can be contacted with a compound, optionally followed by a rinse, and within 10 minutes or less from first application, the compound can be detected specifically in abnormal and disease-state cells. This can permit quick detection of abnormal and disease-state cells and tissues, and thus quick surgical or other treatment thereof. In some embodiments, these advantageous properties of the compounds also make possible combined treatment and diagnosis, either with a mixture of compounds with the mixture exhibiting both types of fluctions, or with a compound that exhibits both.

The chelates in one embodiment can exhibit significantly increased uptake, compared to normal cells, e.g., within 5 minutes and/or within 2-3 hours of administration. In addition, compounds can exhibit unexpectedly high cellular residence times. As a result, the compounds in some embodiments are suited to surfacial methods, include topical administration, and/or surface-accessible detection and treatment methodologies including, e.g., surgical scission, excision, or ablation.

Compositions and compounds which may be used for diagnostic or therapeutic purposes may be administered as an IV injection formulation. Alternatively, and equally acceptable, the compositions and compounds as described herein can be topically applied, for example, in some embodiments, for use as optical dyes or markers for diseased or “perturbed” tissues.

1. Formulations

The compounds disclosed herein may be employed in a variety of formats and formulations. These may be used as the sole active, therapeutic or diagnostic ingredient or they may be mixed with other active ingredients, as well. Useful formats include, e.g., solutions, suspensions, emulsions, slurries, pastes, creams, gels, foams, and the like, presented in any useful configuration, e.g., capsules, ampoules, ointments, sprays, mists, aerosols, and the like. In some embodiments, frozen, lyophilized, and/or powdered formulations may be employed.

The compounds can be administered in the form of a pharmaceutical composition. However, such a material according to the present invention can be administered or applied alone, or it can be applied, e.g., in vitro, in the form of a composition that is acceptable for only in vitro, not in vivo, administration, such as where a composition is applied to an isolated cell, tissue, or biomolecule sample.

A pharmaceutical composition hereof will contain a compound disclosed herein together with one or more of pharmaceutically acceptable other active ingredients, excipients, buffers, solvents, lubricants, carriers, preservatives, stabilizers, diluents, fillers, or other ingredients known in the art. For example, see A. R. Gennaro (ed.), Remington: The Science and Practice of Pharmacy, 20^{th} ed. (2000) (Lippincott, Williams & Wilkins); A. H. Kibbe et al. (eds.), Handbook of Pharmaceutical Excipients, 4^{th} ed. (May 2003) (Pharmaceutical Press); and U.S. Pat. Nos. 6,710,065 and 6,664,269. As is commonly understood in the art, the term “pharmaceutically acceptable” as used herein includes materials and concentrations that are recognized, in sound medical or veterinary judgment, to be suitable for in vivo or ex vivo administration to (respectively) a subject human or animal, without excessive allergic, toxic, or other complicating response, as balanced with consideration of the benefit to be obtained by the administration thereof. In addition, as is commonly understood in the art, a pharmaceutical composition will also be pharmaceutically acceptable in that the active ingredient(s), excipients, diluents, and so forth, selected for combination in making the formulation, will be compatible with one another.

The formulation may be prepared by any method known in the art, for example, contacting the active ingredient(s) with one or more other ingredients, preferably in a solvent or liquid carrier, more preferably with substantially uniform mixing of the ingredients therein. This may be followed by, e.g., drying, lyophilizing, or freezing; or by further compounding to form, e.g., an emulsion, cream, paste, or the like. In a preferred embodiment, the formulation prepared will be presented in unit dosage form for use.

The effective compound, including a chelant or chelate can be used in the form of pharmaceutically acceptable salts derived from inorganic or organic acids. By “pharmaceutically acceptable salt” is meant those salts which are suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zürich, Switzerland: 2002). The salts can be prepared in situ during the final isolation and purification of the compounds of the present invention or separately by reacting a free base function with a suitable organic acid. Basic addition salts can be prepared in situ during the final isolation and purification of compounds by reacting a carboxylic acid-containing moiety with a suitable base.

For the purpose of the present invention, the complexes described herein and physiologically acceptable salts thereof are considered equivalent in the therapeutically effective compositions. Physiologically acceptable salts refer to the acid addition salts of those bases which will form a salt with at least one acid group of the ligand employed and which will not cause a significant adverse physiological effect when administered to a mammal at dosages consistent with good pharmacological practice. Suitable bases include, for example, the alkali metal and alkaline earth metal hydroxides, carbonates, and bicarbonates such as sodium hydroxide, potassium hydroxide, calcium hydroxide, potassium carbonate, sodium bicarbonate, magnesium carbonate, ammonia, primary, and secondary and tertiary amines. Physiologically acceptable salts may be prepared by treating the acid with an appropriate base.

The formulations may conveniently be presented in unit dosage form and may be prepared by any of the methods well known in the art of pharmacy. The compound or a pharmaceutically acceptable salt thereof (“active ingredient”) can be combined with the pharmaceutical carrier which constitutes one or more accessory ingredients.

The compound can be mixed with other active materials that do not impair the desired action, or with materials that supplement the desired action. Solutions or suspensions used for parenteral, intradermal, subcutaneous, or topical application can include, for example, the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic. If administered intravenously, preferred carriers are physiological saline or phosphate buffered saline (PBS). Depot injectable formulations are also prepared by entrapping the drug in liposomes or microemulsions which are compatible with body tissues.

2. Administration

The compounds are preferably administered by any appropriate administration route, for example, orally, parenterally, intravenously, intradermally, intramuscularly, subcutaneously, sublingually, transdermally, bronchially, pharyngolaryngeal, intranasally, topically such as by a cream or ointment, rectally, intraarticular, intracistemally, intrathecally, intravaginally, intraperitoneally, intraocularly, by inhalation, bucally or as an oral or nasal spray. The route of administration may vary, however, depending upon the condition and the severity of the condition. The precise amount of compound administered to a host or patient will be the responsibility of the attendant physician. However, the dose employed will depend on a number of factors, including the age and sex of the patient, the precise disorder being treated, and its severity.

Exemplary dose ranges include: from about 0.001 mg/kg per day to about 2500 mg/kg per day; from about 0.1 mg/kg per day to about 1000 mg/kg per day; and from about 0.1 mg/kg per day to about 500 mg/kg per day, including 1 mg/kg, 2 mg/kg, 5 mg/kg, 10 mg/kg, 15 mg/kg, 20 mg, kg, 25 mg/kg, 30 mg/kg, 35 mg/kg, 40 mg/kg, 45 mg/kg, 50 mg/kg, 100 mg/kg, 200 mg/kg, 300 mg/kg, 400 mg/kg, 500 mg/kg per day, and values between any two of the values given in this range. The dose range for humans can be, e.g., from about 0.005 mg to 100 g/day.

In another embodiment, the dose range is such that the blood serum level of compounds is from about 0.01 μM to about 100 μM, or from about 0.1 μM to about 100 μM. Exemplary values of blood serum levels include but are not limited to about 0.01 μM, about 0.1 μM, about 0.5 μM, about 1 μM, about 5 μM, about 10 μM, about 15 μM, about 20 μM, about 25 μM, about 30 μM, about 35 μM, about 40 μM, about 45 μM, about 50 μM, about 55 μM, about 60 μM, about 65 μM, about 70 μM, about 75 μM, about 80 μM, about 85 μM, about 90 μM, about 95 μM and about 100 μM, as well as any blood serum level that falls within any two of these values (e.g, between about 10 μM and about 60 μM). Tablets or other forms of dosage presentation provided in discrete units may conveniently contain an amount of one or more of the compounds of the invention which are effective at such dosage rages, or ranges in between these ranges.

In general, the compounds, or pharmaceutically acceptable salts thereof, will be administered to a host so that a therapeutically effective amount is received. A therapeutically effective amount may conventionally be determined for an individual patient by administering the active compound in increasing doses and observing the effect on the patient, for example, reduction of symptoms associated with the particular condition. Generally, the compound must be administered in a manner and a dose to achieve in the human the desired blood level concentration of a compound needed to exhibit a therapeutic effect.

Compounds may be administered in the form of liposomes. Any non-toxic, physiologically acceptable and metabolizable lipid capable of forming liposomes may be used. The present compositions in liposome form may contain, in addition to the compounds, stabilizers, preservatives, excipients, and the like.

The compounds and formulations of the present invention can be administered in any of the known dosage forms standard in the art, including solid dosage form, semi-solid dosage form, and liquid dosage form.

Solid dosage forms for oral administration include capsules, caplets, tablets, pills, powders, lozenges, and granules. Dosage forms for topical or transdermal administration of a compound include ointments, pastes, creams, lotions, gels, powders, solutions, sprays, inhalants or patches, optionally mixed with degradable or nondegradable polymers.

The formulations may be, e.g., topical or injectable formulations. In one embodiment, for topical applications, the chelate is formulated at a concentration of 1 μM to 10 mM in an aqueous solution. In one embodiment, for injectable application, the chelate is administered at 0.001-0.2 mmol/kg of body weight.

VIII. Specific Protein Binding

The chelates have been unexpectedly found to tightly and specifically bind to a target protein within abnormal and disease state cells. Though not wishing to be bound by theory, it is believed that this unexpected feature may serve to help increase the very long residence time of the chelants and chelates in, e.g., cancer cells. Further unexpected is that, unlike most reported chelates in the literature, these chelates in one embodiment are distributed substantially evenly throughout the cytoplasm of cells, not solely, or not even mainly, on the cell membrane.

This unexpected tight, specific binding was further elucidated as follows. This feature was unexpectedly identified upon performing polyacrylamide gel electrophoresis (PAGE) of cell contents. One particular protein band was found to retain a fluorescent chelate associated therewith, even though the PAGE gel was a reducing gel (in which the samples had been treated with either beta-mercaptoethanol, or dithiothreitol). The protein band (of about 15 kD) was isolated and fragmented, and the peptide fragments were sequenced by mass spectrometry. The resulting data was used to search GenBank, wherein the protein was identified as a human, hypothetical, membrane-associated protein, HSPC194 (see e.g., GenBank Accession No. AF151028; PCT Publication Nos. WO 01/036684, WO 99/040189 and WO 03/054152), a protein that had been postulated from analysis of genomic DNA and mRNA/cDNA, but that apparently had not yet been isolated or characterized. In light of the fact that this is a hypothetic membrane-associated protein, it is particularly surprising that the chelate-protein complex was isolated from the cytosolic protein fraction, rather than from a membrane-associated cell fraction, and that the fluorescent chlelate bound to the protein, is observed throughout the cytosol, rather than localized upon any membrane-bound cell structure.

Thus, chelates have been discovered to have specific binding properties for proteins in abnormal cells, in particular specific diseased cells such as cancerous or apoptotic cells with perturbed membranes that undergo selective increased uptake of the chelates. As used herein, the term “perturbed membrane” means a membrane demonstrating morphological and molecular changes, including for example, membrane blebbing (weakening of the membrane), scrambling, and redistribution of aminophospholipids and the like). For example, as described in the herein, Tb-PCTMB has been determined to selectively bind to the protein of Seq ID No.:2 expressed in cancer cell lines. Without being limited to any theory, this selective binding to a protein may in part assist in prolonging the duration of uptake of the chelate in the cell, thus enhancing the residence time of the chelate in the cancer cell.

In one embodiment, there is provided a method of identifying a protein to which a chelate specifically binds, by screening for the protein in an abnormal cell, and in particular, a diseased cell (e.g., a cancer cell). Screening can be conducted by methods known in the art, such as using immobilized arrays. For example, arrays of a proteins can be contacted with detectable chelates. The binding of a chelate also can be detected by methods such as chromatography after treatment of a culture of a cancer line by the chelate, and detecting uptake of the chelate into the cell.

Moreover, methods are provided for the use of such proteins to which chelates specifically bind. For example, proteins, and nucleic acids encoding the proteins, identified in cancer cells can be used as diagnostic indicators of the presence of particular disease states. In one embodiment, molecules that specifically bind to the proteins, or nucleic acids encoding the proteins, can be used in diagnostic and therapeutic applications. Additionally, methods of screening the efficacy of a chelate in diagnostic or therapeutic applications are provided, wherein binding of the chelate to such proteins associated with abnormal cells is determined.

In a particular embodiment, methods for diagnosis and therapy are provided, wherein the protein associated with a disease state is SEQ. ID NO.: 2, or 5, 7, 9 or 10. In one embodiment, a method of diagnosing a disease in a host is provided, the method comprising administering to the host a detectable molecule capable of binding to a protein having the sequence of SEQ. ID NO.:2 or 5, 7, 9 or 10, and detecting the binding of the molecule to the protein in a diseased cell. In one particular embodiment, the detectable molecule is a chelant, such as Tb-PCTMB or Eu-PCTMB.

Also provided is an isolated non-covalent complex of a compound, such as those described herein, and a polypeptide having an amino acid sequence of SEQ ID NO: 2 or 5, 7, 9 or 10.

In another embodiment, a method of evaluating the efficacy of a compound as a therapeutic or diagnostic agent is provided, the method comprising screening the compound for ability to bind to a protein of SEQ. ID No. 2 or 5, 7, 9 or 10 or a fragment thereof having for example at least 20 amino acids. The compound is, e.g., a compound as disclosed herein.

Also provided are arrays useful for identifying at least one target material that specifically binds to the polypeptide of SEQ ID NO: 2 or 5, 7, 9 or 10, wherein the array comprises a plurality of zones on or in which are immobilized at least one polypeptide of SEQ ID NO: 2 or 5, 7, 9 or 10 or fragment thereof, e.g. having at least 20 amino acids, or e.g., from about 20 to 100 amino acids.

Also provided are arrays useful for identifying disease state cells or abnormal cells susceptible to in vivo detection or treatment by an azamacrocycle compound, e.g., as described herein, wherein the array comprises a plurality of zones on or in which are immobilized at least one polynucleotide or polynucleotide analog having the base sequence of SEQ ID NO: 1, 6, 8 or other sequence encoding a polypeptide of SEQ ID No. 2, 5, 7, 9 or 10, or an oligonucleotide or oligonucleotide analog having a base sequence of about 10, 20, 30 or more contiguous bases thereof.

Optionally in other embodiments, other polypeptides and oligonucleotides which bind specifically to the compounds as disclosed herein can be used as described herein in assays and formulations based on their specific binding properties.

For example, the following compounds 2 and 3 were found to bind to BIP (Seq. ID No. 5, Immunoglobulin heavy chain binding protein, Rasmussen, R. K., et al., 1997, “Two-dimensional electrophoretic analysis of human breast carcinoma proteins: mapping of proteins that bind to the SH3 domain of mixed lineage kinase MLK2”, Electrophoresis 18 (3-4), 588-598; and Ji, H., et al., 1997, “A two-dimensional gel database of human colon carcinoma proteins,” Electrophoresis 18 (3-4), 605-613). Compounds 2 and 3 were also found to bind to amphiphysin I (Seq. ID No. 7, see Floyd et al., “Expression of amphiphysin I, an autoatigen of paraneoplastic neurological syndromes in breast cancer”, Mol. Med. (Cambridge, Mass.) (1998). 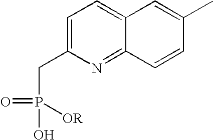
Compound 2, R=Et
Compound 3, R=n-Butyl

The following compounds 8, 9 and 12 were found to bind to cytokeratin 8 (Seq. ID No. 10) and keratin 18 (Seq. ID No. 9). See Leube, R. E., et al., 1986, “Cytokeratin expression in simple epithelia. III. Detection of mRNAs encoding human cytokeratins nos. 8 and 18 in normal and tumor cells by hybridization with cDNA sequences in vitro and in situ”, Differentiation 33 (1), 69-85. 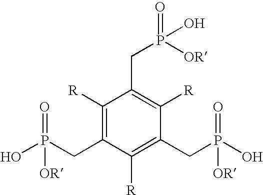
Compound 8, R=H, R₁=Et
Compound 9, R=H, R₁=n-Butyl
Compound 12, R=Me, R₁=n-Butyl.

Thus, in one embodiment, provided is an isolated compound for example of Formula I, Ia or II complexed with a polypeptide having an amino acid sequence of SEQ ID NO. 2, 5, 7, 9 or 10.

Further provided is a method of diagnosing or treating a disease in a host comprising administering to a host or tissue or cell sample there from a protein or other compound for example of Formula I, Ia or II capable of complexing with a protein having the sequence of SEQ ID NO. 2, 5, 7, 9 or 10.

Also provided is a method of evaluating the efficacy of a compound for example of Formula I, Ia or II as a therapeutic or diagnostic agent, the method comprising screening the compound for ability to bind to a protein of either SEQ. ID No. 2, 5, 7, 9 or 10, or a fragment thereof optionally having at least 20 amino acids.

IX. Kits and Uses

In one embodiment, the compounds, complexes and conjugates described herein may be provided in the form of a kit in which a sample of compound, e.g. chelant, or chelate or of a composition containing the same is located, with instructions for its use. The sample may be a solution or suspension or a frozen, dried, or lyophilized preparation. The preparation may contain the compound in the form of a salt, or the preparation may be salt-free. The preparation may optionally contain a pH buffering agent, preferably a pharmaceutically acceptable pH buffering agent, either as part of an aqueous medium in a solution or suspension, or else as an admixed solid, e.g., a powdered or granular buffering agent; or a pH buffering agent may be separately provided in the kit as a solid or dissolved buffering agent located in a separate packet or container. In one embodiment of a sample already prepared for administration, the sample will be an otherwise salt-free solution or suspension of a chelant, chelate, or conjugate in a pharmaceutically acceptable, buffered, aqueous medium.

Instructions in the kit may include directions for how to further prepare the sample, as by thawing and/or diluting it, and/or by forming a solution or suspension therefrom, directions for how to prepare a chelate from a chelant structure in the sample, and/or directions for how to prepare a complex.

Instructions may also include directions for diagnostic or therapeutic uses, and these may include directions as to how to apply or administer the sample or a composition prepared therefrom to, e.g., a cell or tissue either in vivo or in vitro (for example, in vitro application to a sample located on a slide, or application to a cell, tissue, or biomolecule array or microarray).

Instructions may also include directions for performing a test following administration of the chelant, chelate, or composition to cells or tissues, e.g.: affinity chromatography, as to enrich a cell sample in abnormal cells; cell-sorting, as by fluorescence-based cell sorting; or application to an affinity molecule array or microarray. The kit may also or alternatively provide an array or microarray of one or more of compounds, with instructions for use, e.g., to identify cells that specifically bind or uptake a chelant or chelate. In such an array or microarray, the cells that specifically interact with the compounds may be visualized by microscopy or detected by an immunodetection technique.

In such an array or microarray embodiment, the compounds may be tethered to the surface of a slide; the structure providing the tether may be selected to be cleavable by the cell, once the tethered compound has been taken up thereby.

Cells bound to the array or microarray may also be indirectly visualized by treating the array, after washing to remove non-specifically bound cells, with an antibody or antibodies to the compound, following by ELISA-based immunodetection thereof. In such an embodiment, the zones lacking an ELISA response will indicate specifically bound cells.

Alternatively two washing steps may be used, the second to wash away specifically bound cells, which carry off with them at least some of the compounds. In such an embodiment, the compound or conjugate structure will preferably be independently detectable, as by exhibiting inherent fluorescence or by having attached thereto or incorporated therein a reporter or other independently detectable structure, e.g., a fluorescent group. In such an embodiment, the decreased fluorescence or other reporter signal of a zone will indicate that a cell has specifically taken up the chelate, or chelant, that had been placed in that zone. In such an embodiment, preferably only one cell type or cell line is tested per microarray.

The chelant, or chelate or other compound as disclosed herein may itself be used as a reporter or marker, e.g., a fluorescent reporter or marker. For example, a microarray of a nucleic acid (e.g., cDNA) library may be screened by suffuse the array with an antisense oligonucleotide to which a fluorescent chelant, or chelate, is attached. After washing to remove any non-specifically bound oligos, the degree of fluorescence or other signal in each zone provides a direct and quantitative measure of oligo binding. Such an embodiment may be applied to the field of individualized medicine, providing a way to, e.g., quickly identify polymorphisms (as single nucleotide polymorphisms), and/or to permit efficient, patient-specific selection of a nucleic acid or other agent to be administered to the patient (e.g., a nucleic acid for anti-sense treatment, gene therapy, etc.).

Compounds and compositions disclosed herein can be used in diagnosis and/or therapy of a variety of abnormal and disease-state cells and tissues. These include cancerous, apoptotic, pre-cancerous, and pre-apoptotic cells and tissues. Soft tissue cancers and pre-cancerous soft-tissues are particularly susceptible of treatment and/or diagnosis thereby. Thus, instructions provided with a kit may state directions as to which are the specific condition(s) or disease(s) the materials of the kit are useful in diagnosis and/or therapy.

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention.

### EXAMPLES

### Example 1

### Synthesis of N-(6-methyl-2quinolylmethyl)-N′,N″,N′″-tris(methylene phosphonic acid ethyl ester)-1,4,7,10-tetraazacyclododecane (6, R¹=quinolyl, R²=C₂H₅, R³=methyl) (QCTME Chelant)

To a stirring solution of N-(6-methyl-2-quinolylmethyl)-1,4,7,10-tetraazacyclododecane (4) (1 g, 0.00305 mol) in dry THF (50 mL) under N₂ was added paraformaldehyde (0.276 g, 0.00918 mol). The reaction was allowed to stir for 3 hours at room temperature. Triethyl phosphite (1.524 g, 0.00918 mol) was then added to the mixture and allowed to stir until the solution turned completely clear. The completed reaction mixture was concentrated and dried under high vacuum for 24 hours to afford a pale yellow oil. The oil was then refluxed for four days with 27 equivalents of KOH dissolved in 20 mL of H₂O with enough dioxane to achieve solubility. The resulting mixture volume was then reduced under vacuum to produce a thick oil. The oil was then washed with a series of increasing chloroform concentration methanol/chloroform solutions with filtration and removal of solvent. The resulting oil was then dissolved in a minimal amount of chloroform and acetonitrile was then added until the solution became cloudy. The mixture was allowed to stand to precipitate the pure product which was then filtered, dissolved in water, and lyophilized to produce 0.520 g (21%) of a slightly yellow, solid. H¹ NMR (D₂O): δ 0.87 (t, 6H), 1.07 (t, 3H), 2.45 (s, 3H), 2.49-3.09 (br m, 25H), 3.47 (p, 4H), 3.76 (p, 2H), 3.89 (s, 2H), 7.55 (m, 3H), 7.76 (d, 1H), 8.15 (d, 1H).

### Example 2

### Preparation of Europium 3,6,9-tris(methylene phosphonic acid n-butyl ester)-3,6,9,15-tetraaza-bicyclo[9.3.1]pentadeca-1(15),11,13-triene (Eu-PCTMB chelate)

The potassium salt of PCTMB (150 mg, 0.19 mmol) was dissolved in deionized water (3 mL) to give a solution of pH 10.5. The pH was lowered to 5.5 using 1N HCl with continuous stirring. An aqueous solution (3 mL) of europium chloride hexahydrate (85.5 mg, 0.23 mmol) was then added in one portion to give a solution having a pH of 3.47. The pH was slowly raised by adding 0.1 mL aliquots of 0.1N KOH. Addition of KOH was terminated when a pH of 6.4 was sustained. At this point the homogeneous solution became soapy and considerable turbidity was observed. The turbid solution was then freeze dried and the resulting solid dissolved in chloroform:methanol (3:1, 40 mL). This organic solution was filtered through CELITE (diatomaceous earth, from Celite Corp., available from World Minerals Inc., Lompoc, Calif., USA) and concentrated to give a glassy solid. The solid was redissolved in water (20 mL), filtered through a 0.2μ filter and freeze dried to give the complex as a flaky, snow white solid. The complex was isolated as a flocculant, off-white solid. The complexation was assessed by HPLC and the yield was quantitative.

### Example 3

### Preparation of Terbium 3,6,9-tris(methylene phosphonic acid n-butyl ester)-3,6,9,15,tetraaza-bicyclo[9.3.1]pentadeca-1(15),11,13-triene (Tb-PCTMB chelate)

The potassium salt of PCTMB (150 mg, 0.19 mmol) was dissolved in deionized water (3 mL) to give a solution of pH 10.5. The pH was lowered to 5.5 using 1N HCl with continuous stirring. An aqueous solution (3 mL) of terbium chloride hexahydrate (85.5 mg, 0.23 mmol) was then added in one portion to give a solution having a pH of 3.47. The pH was slowly raised by adding 0.1 mL aliquots of 0.1N KOH. Addition of KOH was terminated when a pH of 6.4 was sustained. At this point the homogeneous solution became soapy and considerable turbidity was observed. The turbid solution was then freeze dried and the resulting solid dissolved in chloroform:methanol (3:1, 40 mL). This organic solution was filtered through CELITE (diatomaceous earth) and concentrated to give a glassy solid. The solid was redissolved in water (20 mL), filtered through a 0.2μ filter and freeze dried to give the complex as a flaky, snow white solid. The complex was isolated as a flocculant, off-white solid. The complexation was assessed by HPLC and the yield was quantitative.

### Example 4

### Preparation of Eu-QCTME Chelate

The potassium salt of N-(6-methyl-2-quinolylmethyl)-N′,N″,N′″-tris(methylene phosphonic acid butyl (R²=n-butyl) or ethyl (R²=ethyl) ester)-1,4,7,10 tetraazacyclododecane (6) (300 mg) was dissolved in 100 mL of distilled water. The pH of the solution, which was around 10.5 to start, was then adjusted to 6.5 using dilute hydrochloric acid. Europium chloride hexahydrate (1 equivalent) was dissolved in 50 mL of distilled water and added to the ligand solution drop-wise with stirring. As the pH began to drop, it was maintained around six with a dilute potassium hydroxide solution. Addition of potassium hydroxide was terminated after all the europium salt had been added and when the pH had settled around 6.4. The solution was then lyophilized, re-dissolved in chloroform and filtered through CELITE (diatomaceous earth). The resulting filtrate was then concentrated producing a glassy solid. The solid was then taken up in water and filtered through a micro-filter to remove Eu(OH)₃ and lyophilized to produce a flocculant white solid.

### Example 5

### Preparation of (6-Methyl-naphthalen-2-ylmethyl)-phosphonic acid monomethyl ester (1)

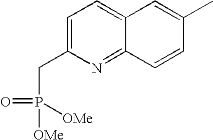

n-Butyl lithium (8.5 ml of a 2.5 M solution in heptane, 21.3 mmol) was added dropwise to a stirred solution of diisopropylamine (2.5 ml, 19.9 mmol) in THF (10 ml) at −20° C. The resulting solution was kept at −20° C. for 1 hour, then cooled to −30° C. before the dropwise addition of a solution of 2,6-dimethylquinoline (2.50 g, 15.9 mmol) in THF (10 ml). The reaction was stirred at −30° C. for a further 1.5 hours then cooled to −50° C. before the dropwise addition of dimethyl chlorophosphate (2.1 ml, 16.7 mmol). The reaction was allowed to warm to −10° C. over 2 hours. Saturated aqueous ammonium chloride solution (40 ml) was added and the reaction was extracted into ethyl acetate (3×30 ml). The combined organic extracts were dried (Na₂SO₄) and concentrated in vacuo. The residue was purified by flash column chromatography (eluent: ethyl acetate/methanol 96:4) to provide the title compound as a yellow solid (1.67 g, 40%).

¹H NMR (400 MHz, CDCl₃) δ ppm 8.02 (1H, d, J 9 Hz), 7.94 (1H, d, J 9 Hz), 7.56-7.52 (2H, m), 7.47 (1H, d, J 9 Hz), 3.73 (6H, d, J 11 Hz), 3.60 (2H, d, J 22 Hz) and 2.53 (3H, s).

³¹P{¹H} NMR (400 MHz, CDCl₃) δ ppm 28.6.

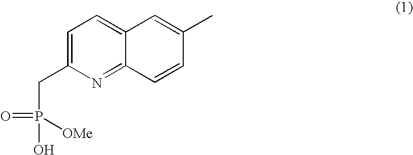

Potassium hydroxide (300 mg, 5.36 mmol) was added to a solution of (28) (755 mg, 2.84 mmol) in water (25 ml) and the reaction was heated to 80° C. After 24 hours the mixture was cooled to room temperature and the pH was adjusted to 6.5 by the dropwise addition of 5% aqueous hydrochloric acid. Toluene (20 ml) was added and the resultant mixture was then concentrated in vacuo. This process was repeated twice to afford the crude product contaminated with potassium chloride as a yellow solid. The solid was dried in a vacuum oven at 40° C. overnight and then slurried in chloroform (10 ml) for 2 hours. The chloroform was filtered and the filtrate was concentrated in vacuo to give the title compound as a white solid (65 mg, 9%).

¹H NMR (400 MHz, CD₃OD) δ ppm 8.14 (1H, d, J 9 Hz), 7.89 (1H, d, J 9 Hz), 7.67-7.65 (2H, m), 7.57 (1H, dd, J 9 and 2 Hz), 3.53 (3H, d. J 11 Hz), 3.42 (2H, d, J 21 Hz) and 2.53 (3H, s).

³¹P{¹H} NMR (400 MHz, CD₃OD) δ ppm 19.9.

### Example 6

### Preparation of (6-Methyl-naphthalen-2-ylmethyl)-phosphonic acid monoethyl ester (2)

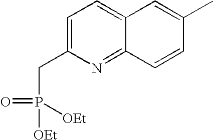

Using the procedure described above for quinoline dimethyl phosphonate analog, n-butyl lithium (8.5 ml of a 2.5 M solution in heptane, 21.0 mmol) was sequentially treated with diisopropylamine (2.5 ml, 19.9 mmol) in THF (10 ml), 2,6-dimethylquinoline (2.5 g, 15.9 mmol) in THF (10 ml) and diethyl chlorophosphate (2.4 ml, 16.7 mmol). Following the same work-up procedure, purification by flash column chromatography (eluent: ethyl acetate/methanol 98:2) provided the title compound as a yellow solid (2.40 g, 51%).

¹H NMR (400 MHz, CDCl₃) δ ppm 8.02 (1H, d, J 9 Hz), 7.93 (1H, d, J 8 Hz), 7.56-7.48 (3H, m), 4.09 (4H, q, J 14.5, 7 Hz), 3.59 (2H, d, J 22 Hz), 2.53 (3H, s) and 1.25 (6H, t, J 7 Hz). ³¹P{¹H} NMR (400 MHz, CDCl₃) δ ppm 26.1.

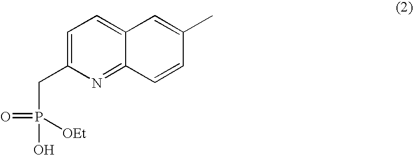

Using the procedure described for (1) above, the diethyl phosphonate(1.00 g, 3.4 mmol) was treated with potassium hydroxide (3.0 g, 53.6 mmol) in water (15 ml) at reflux. After 17 hours, the pH of the reaction was adjusted to 6.5 by the dropwise addition of 5% aqueous hydrochloric acid. The reaction mixture was washed with chloroform (10 ml) then concentrated in vacuo to give the crude product contaminated with potassium chloride. The solid was dried in a vacuum oven at 40° C. overnight, then placed in soxhlet apparatus and extracted with refluxing chloroform. Concentration in vacuo afforded the title compound as a white solid (420 mg, 46%).

¹H NMR (400 MHz, D₂O) δ ppm 7.87 (1H, d, J 8 Hz), 7.60 (1H, d, J 8 Hz), 7.38 (2H, d, 8 Hz), 7.22 (1H, dd, J 8 and 2 Hz), 3.63-3.57 (2H, m), 3.16 (2H, d, J 2 Hz), 2.28 (3H, s) and 0.93 (3H, t, J 7 Hz).

³¹P{¹H} NMR (400 MHz, D₂O) δ ppm 21.0.

### Example 7

### Preparation of (6-Methyl-naphthalen-2-ylmethyl)-phosphonic acid monobutyl ester (3)

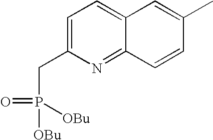

A stirred mixture of tributylphosphite (5.0 ml, 18.3 mmol), and 2-chloromethyl-6-methylquinoline (1.0 g, 5.2 mmol) was heated in an oil bath at 130° C. (external temperature). After 24 hours the mixture was cooled to room temperature and purified by flash column chromatography (eluent: ethyl acetate) to provide the dibutyl phosphonate compound as a yellow oil (1.12 g, 61%).

¹H NMR (400 MHz, CDCl₃) δ ppm 8.02 (1H, d, J 9 Hz), 7.92 (1H, d, J 9 Hz), 7.56-7.48 (3H, m), 4.01 (4H, q, J 13.0, 7 Hz), 3.59 (2H, d, J 22 Hz), 2.53 (3H, s), 1.59-1.53 (4H, m), 1.34-1.26 (4H, m) and 0.84 (6H, t, J 8 Hz).

³¹P{¹H} NMR (400 MHz, CDCl₃) δ ppm 26.0.

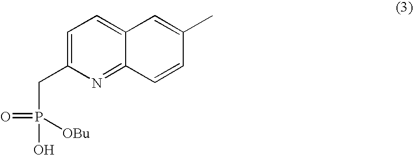

The dibutyl ester (2.78 g, 7.96 mmol) was dissolved in a mixture of 1,4-dioxane (25 ml) and water (25 ml). Potassium hydroxide (3.00 g, 53.6 mmol) was added and the reaction was heated to reflux. After 17 hours the pH of the reaction was adjusted to 6.9 by the dropwise-addition of 5% aqueous hydrochloric acid. The reaction mixture was concentrated in vacuo. Toluene (20 ml) was added to the resultant oil and the solution was then concentrated in vacuo. This process was repeated. The resultant yellow solid was dissolved in anhydrous iso-propanol (15 ml), the solution was filtered and the filtrate was concentrated in vacuo to give an off-white solid. The solid was added to anhydrous ethyl acetate (30 ml) and the resulting suspension was sonicated for 20 minutes. The solid was collected by filtration and dried in vacuo to afford the title compound as a white solid (560 mg, 24%).

¹H NMR (400 MHz, CDCl₃) δ ppm 7.75 (1H, d, J 9 Hz), 7.70 (1H, d, J 9 Hz), 7.28-7.22 (2H, m), 7.14 (1H, dd, J 9 and 2 Hz), 3.65-3.60 (2H, m), 3.24 (2H, d, J 21 Hz), 2.35 (3H, s), 1.31-1.28 (2H, m), 1.05 (2H, sextet, 7 Hz) and 0.63 (3H, t, J 7.5 Hz).

³¹P{¹H} NMR (400 MHz, CDCl₃) δ ppm 18.5.

### Example 8

### Preparation of [6-(Hydroxy-methoxy-phosphorylmethyl)-pyridin-2-ylmethyl]-phosphonic acid monomethyl ester (4)

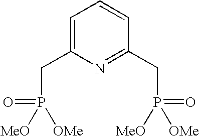

A stirred mixture of trimethylphosphite (8.0 ml, 67.8 mmol), and 2,6-bis(chloromethyl)pyridine (1.00 g, 5.7 mmol) was heated in an oil bath at 130° C. (external temperature). After 24 hours the mixture was cooled to room temperature, purified by flash column chromatography (eluent: ethyl acetate/methanol 90:10) to provide the pyridyl bis-dimethyl phosphonate ester compound as a yellow oil (1.40 g, 76%).

¹H NMR (400 MHz, CDCl₃) δ ppm 7.62 (1H, t, J 8 Hz), 7.28-7.25 (2H, m), 3.73 (12H, d, J 11 Hz) and 3.42 (4H, d, J 22 Hz).

³¹P{¹H} NMR (400 MHz, CDCl₃) δ ppm 26.3.

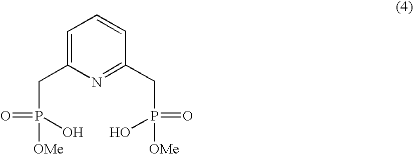

Potassium hydroxide (3.00 g, 53.6 mmol) was added to a solution of the pyridyl full phosphonate ester (1.23 g, 3.81 mmol) in water (15 ml) and the mixture was heated to reflux. After 2 hours the mixture was cooled to room temperature and the pH was adjusted to 6.5 by the dropwise addition of 5% aqueous hydrochloric acid solution. Following filtration the filtrate was concentrated in vacuo and the residue was dissolved in toluene (20 ml). The mixture was then concentrated in vacuo. This process was repeated to provide the crude product which was dissolved in anhydrous iso-propanol (15 ml). The resultant suspension was filtered and the filtrate was concentrated in vacuo to provide the title compound as a white solid (700 mg, 62%).

¹H NMR (400 MHz, D₂O) δ ppm 7.59 (1H, t, J 8 Hz), 7.17-7.14 (2H, m), 3.37 (6H, d, J 11 Hz) and 3.11 (4H, d, J 22 Hz).

³¹P{¹H} NMR (400 MHz, D₂O) δ ppm 23.1.

### Example 9

### Preparation of [6-(Hydroxy-ethoxy-phosphorylmethyl)-pyridin-2-ylmethyl]-phosphonic acid monoethyl ester (5)

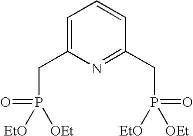

A stirred mixture of triethylphosphite (10.0 ml, 58.3 mmol), and 2,6-bis(chloromethyl)pyridine (1.00 g, 5.7 mmol) was heated in an oil bath at 130° C. (external temperature). After 24 hours the mixture was cooled to room temperature, purified by flash column chromatography (eluent: ethyl acetate/methanol 95:5) to provide the title compound as a yellow oil (1.44 g, 67%).

¹H NMR (400 MHz, CDCl₃) δ ppm 7.60 (1H, t, J 8 Hz), 7.29-7.27 (2H, m), 4.11-4.04 (8H, m), 3.39 (4H, d, J 23 Hz) and 1.27 (12H, t, J 7 Hz).

³¹P{¹H} NMR (400 MHz, CDCl₃) δ ppm 26.3.

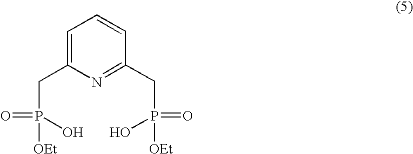

Using the procedure described above for (4), the full phosphonate ethyl ester (1.43 g, 3.79 mmol) was treated with potassium hydroxide (3.00 g, 53.6 mmol) in water (15 ml) for 3 hours to provide the title compound as a white solid (720 mg, 59%).

¹H NMR (400 MHz, D₂O) δ ppm 7.71 (1H, t, J 8 Hz), 7.24 (2H, br d, J 8 Hz), 3.70-3.67 (4H, m), 3.12 (4H, d, J 22 Hz) and 1.00 (6H, t, J 8 Hz).

³¹P{¹H} NMR (400 MHz, D₂O) δ ppm 20.1.

### Example 10

### Preparation of [6-(Hydroxy-butoxy-phosphorylmethyl)-pyridin-2-ylmethyl]-phosphonic acid monobutyl ester (6)

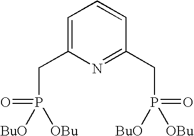

A stirred mixture of tributylphosphite (10.0 ml, 36.6 mmol), and 2,6-bis(chloromethyl)pyridine (1.00 g, 5.7 mmol) was heated in an oil bath at 130° C. (external temperature). After 24 hours the mixture was cooled to room temperature, purified by flash column chromatography (eluent: ethyl acetate/methanol 95:5) to provide the title compound as a yellow oil (1.59 g, 54%).

¹H NMR (400 MHz, CDCl₃) δ ppm 7.58 (1H, t, J 8 Hz), 7.29-7.26 (2H, m), 4.00 (8H, q, J 7 Hz), 3.38 (4H, d, J 23 Hz), 1.62-1.55 (8H, m), 1.39-1.30 (8H, m) and 0.90 (12H, t, J 8 Hz).

³¹P{¹H} NMR (400 MHz, CDCl₃) δ ppm 26.3.

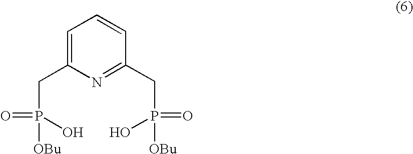

Using the procedure described above for (4), the full n-Bu phosphonate ester (3.35 g, 6.80 mmol) was treated with potassium hydroxide (3.00 g, 53.6 mmol) in water (25 ml) and dioxane for 17 hours to provide the title compound as a white solid (1.20 g, 46%).

¹H NMR (400 MHz, D₂O) δ ppm 7.66 (1H, t, J 8 Hz), 7.23 (2H, br d, J 8 Hz), 3.62 (4H, q, J 6 Hz), 3.13 (4H, d, J 22 Hz), 1.40-1.33 (4H, m), 1.18-1.09 (4H, m) and 0.72 (6H, t, J 7 Hz)

³¹P{¹H} NMR (400 MHz, D₂O) δ ppm 20.9.

### Example 11

### Preparation of 1,3,5-Tris-bromomethyl-benzene

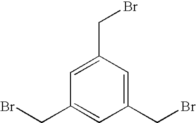

2,2′-Azobis(2-methylpropionitrile) (45 mg, 0.27 mmol) was added to a stirred suspension of N-Bromosuccinimide (115.4 g, 0.64 mol) and mesitylene (25 ml, 0.18 mol) in dichloromethane (500 ml). The solution was warmed to reflux over 20 minutes. After heating at reflux for 3 hours, additional 2,2′-azobis(2-methylpropionitrile) (90 mg, 0.54 mmol) was added to the mixture and heating at reflux was continued for one hour. The suspension was cooled to room temperature and filtered. The filtrate was sequentially washed with half saturated solution of sodium bicarbonate (2×500 ml) and water (500 ml), dried (Na₂SO₄), filtered and concentrated in vacuo. Partial purification by flash colunm chromatography (eluant: heptane) provided two fractions which were further purified independently.

The minor fraction was dissolved in ethyl acetate and heptane was added to induce crystallisation (3.75 v/w, 3.3:1) to provide, after stirring for 2 hours at room temperature, the title compound as a white powder (1.39 g, 2%). The filtrate was concentrated in vacuo, dissolved in ethyl acetate and heptane was added to induce crystallisation (5.80 v/w, 4.4:1) to provide a further crop of the title compound as a white powder (571 mg, 1%). The major fraction was treated in a similar manner using ethyl acetate/heptane (3.5 v/w, 3.75:1) to afford the title compound as a white powder (894 mg, 1%). Two further crops were obtained from the filtrate using the same crystallisation protocol (3.38 g, 5% and 2.53 g, 4%).

¹H NMR (400 MHz, DMSO-d₆) δ ppm 7.35 (3H, s) and 4.45 (6H, s).

### Example 12

### Preparation of [3,5-Bis-(Methoxy-hydroxy-phosphorylmethyl)-benzyl]-phosphonic acid monomethyl ester (7)

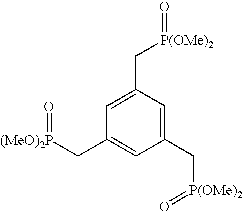

A solution of the 1,3,5 tris benzyl bromide(1.33 g, 3.70 mmol) in trimethylphosphite (5.0 ml, 42.3 mmol) was heated at reflux for 16 hours. The reaction mixture was then cooled down to room temperature and concentrated in vacuo. Ethyl acetate (10 ml) was added to the residue and crystallisation occurred within 5 minutes at room temperature. After stirring at room temperature for 2 hours, the precipitate was filtered to give the title compound as an off-white powder (711 mg, 43% yield).

¹H NMR (400 MHz, CDCl₃) δ ppm 7.13 (3H, d, J 2 Hz), 3.67 (18H, d, J 10 Hz) and 3.13 (6H, d, J 21 Hz).

³¹P{¹H} NMR (161.97 MHz, CDCl₃) oppm 29.7.

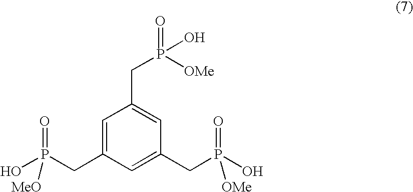

A solution of the 1,3,5-tris bismethyl phosphonate ester (720 mg, 1.62 mmol) and potassium hydroxide (1.30 g, 23.1 mmol) in water (15 ml) was heated at reflux for 17 hours. The reaction mixture was cooled to room temperature, acidified to pH 1 using concentrated hydrochloric acid and then concentrated in vacuo. Methanol (10 ml) was added to the residue and the resultant suspension was stirred for 3 hours. The precipitate was filtered and washed with methanol (10 ml). The filtrate was concentrated in vacuo and the residue was dissolved in water (10 ml) and filtered through Dowex 50XW8-200 ion exchange resin (˜90 ml) (eluant: water). Concentration in vacuo provided the crude product which was slurried in iso-propanol (10 ml) at room temperature for 90 minutes before being filtered to afford the title compound as a white powder (481 mg, 74% yield) Microanalysis indicated <0.1% Cl present.

¹H NMR (400 MHz, DMSO-d₆) δ ppm 7.06 (3H, s), 3.55 (9H, d, J 11 Hz) and 3.04 (6H, d, J 21 Hz).

³¹P{¹H} NMR (161.97 MHz, DMSO-d₆) δ ppm 25.6.

¹³C NMR (100.6 MHz, DMSO-d₆) δ ppm 32.6, 32.9, 52.0, 129.4 and 133.2.

### Example 13

### Preparation of [3,5-Bis-(Ethoxy-hydroxy-phosphorylmethyl)-benzyl]-phosphonic acid monoethyl ester (8)

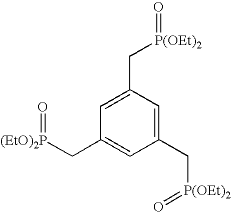

A solution of the 1,3,5-tris benzyl bromide (1.00 g, 2.81 mmol) in triethylphosphite (5.0 ml) was heated at reflux under nitrogen for 17 hours. The mixture was concentrated in vacuo and the residue purified by flash column chromatography (eluant: methanol/ethyl acetate 5:95 to 1:9 gradient) to provide the title compound as a colourless oil (534 mg, 49%).

¹H NMR (400 MHz, CDCl₃) δ ppm 7.14-7.12 (3H, m), 4.06-3.99 (12H, m), 3.11 (6H, d, J 22 Hz) and 1.25 (18H, t, J 7 Hz).

³¹P{¹H} NMR (162 MHz, CDCl₃) δ ppm 26.9.

¹³C NMR (100.6 MHz, CDCl₃) δ ppm 16.4, 32.8, 34.2, 62.1, 129.8 and 132.2.

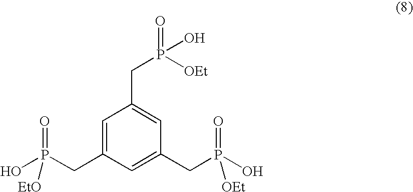

A solution of 1,3,5-tris bisethyl phosphonate ester (347 mg, 0.65 mmol) and potassium hydroxide (761 mg, 13.5 mmol) in water (15 ml) was heated at reflux for 16 hours. The reaction mixture was cooled to room temperature and then acidified to pH 1 using concentrated hydrochloric acid. The resultant solution was extracted with dichloromethane (2×25 mL). The organic extracts were dried (Na₂SO₄), filtered and concentrated in vacuo to provide the title compound as a colorless oil. The aqueous layer was concentrated in vacuo and the residue was slurried in methanol (8 ml) for 2 hours. The precipitate was filtered off, washed with methanol (8 ml) and the filtrate was concentrated in vacuo. The combined organic extracts were dissolved in water (10 ml) and filtered through Dowex 50XW8-200 ion exchange resin (˜80 ml) (eluant: water). Following concentration in vacuo the residue was slurried in iso-propanol (10 ml) at 50° C. for 6 hours. Filtration afforded the title compound as a white powder (120 mg, 41% yield). Microanalysis indicated 0.35% Cl content.

¹H NMR (400 MHz, DMSO-d₆) δ ppm 7.02 (3H, d, J 2 Hz), 3.91-3.84 (6H, m), 2.97 (6H, d, J 21 Hz) and 1.15 (9H, t, J 7 Hz).

³¹P{¹H} NMR (162.0 MHz, DMSO-d₆) δ ppm 24.5.

¹³C NMR (100.6 MHz, DMSO-d₆) δ ppm 16.7, 33.2, 34.5, 60.9, 129.4 and 133.2.

### Example 14

### Preparation of [3,5-Bis-(Butoxy-hydroxy-phosphorylmethyl)-benzyl]-phosphonic acid monobutyl ester (9)

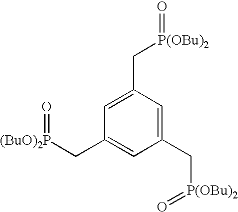

A solution of tris benzyl bromide(891 mg, 2.49 mmol) in tributylphosphite (5 ml, 18.2 mmol) was heated in an oil bath at 130° C. for 16.5 hours. The reaction mixture was cooled to room temperature and then concentrated in vacuo. Purification by flash column chromatography (eluent: ethyl acetate/heptane 1:1 to ethyl acetate to methanol/ethyl acetate 5:95 gradient) afforded the title compound as a pale yellow liquid (1.68 g, 96% yield).

¹H NMR (400 MHz, CDCl₃) δ ppm 7.12 (3H, d, J 2 Hz), 3.96-3.91 (12H, m), 3.09 (6H, d, J 21 Hz), 1.61-1.54 (12H, m), 1.39-1.29 (12H, m) and 0.90 (18H, t, J 7 Hz).

³¹P{¹H} NMR (161.97 MHz, CDCl₃) δ ppm 27.2.

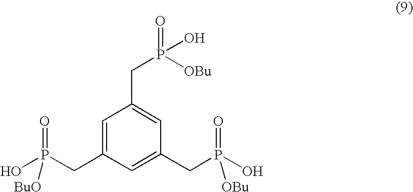

A solution of 1,3,5-tris bis-n-butyl phosphonate ester (1.68 g, 2.4 mmol), potassium hydroxide (2.51 g, 44.7 mmol) and dioxane (30 ml) in water (30 ml) was heated at reflux for 19 hours. The reaction mixture was cooled to room temperature, acidified to pH 1 using concentrated hydrochloric acid and then extracted with dichloromethane (2×50 ml). The extracts were dried (Na₂SO₄) and concentrated in vacuo to provide the title compound as a white powder (1.23 g, 96% yield).

¹H NMR (400 MHz, DMSO-d₆) δ ppm 7.02 (3H, d, J 2 Hz), 3.81 (6H, q, J 6 Hz), 2.96 (6H, d, J 21 Hz), 1.53-1.46 (6H, m), 1.34-1.25 (6H, m) and 0.86 (9H, t, J 7 Hz).

³¹P{¹H} NMR (162 MHz, DMSO-d₆) δ ppm 24.3.

¹³C NMR (100.6 MHz, DMSO-d₆) δ ppm 13.9, 18.6, 32.5, 33.1, 34.5, 64.5, 129.4 and 133.2.

### Example 15

### Preparation of [3,5-Bis-(Methoxy-hydroxy-phosphorylmethyl)-2,4,6-trimethyl-benzyl]-phosphonic acid monomethyl ester (10)

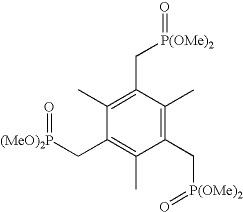

A solution of 2,4,6-tris methyl-1,3,5-tris benzyl bromide (3.64 g, 9.10 mmol) in trimethylphosphite (12.0 ml, 102 mmol) was heated at reflux for 17 hours. The reaction mixture was cooled to room temperature and then concentrated in vacuo. Ethyl acetate (30 ml) was added to the residue and crystallisation occurred within 30 minutes at room temperature. After stirring at room temperature for 3 hours, the precipitate was filtered off to provide the title compound as a white powder (1.95 g, 44%). A second crop was obtained from the liquors by recrystallisation from ethyl acetate (15 ml) as a white powder (725 mg, 16%)

¹H NMR (400 MHz, CDCl₃) δ ppm 3.62 (18H, d, J 10 Hz), 3.36 (6H, d, J 24 Hz) and 2.43 (9H, s).

³¹P{¹H} NMR (162 MHz, CDCl₃) δ ppm 30.7.

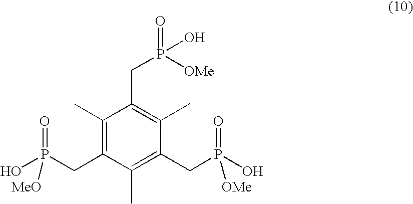

A solution of 2,4,6-tris methyl-1,3,5-tris benzyl dimethylphosphonate ester (1.15 g, 2.30 mmol) and potassium hydroxide (3.47 g, 61.8 mmol) in water (40 ml) was heated at reflux for 17 hours. The reaction mixture was cooled to room temperature, acidified to pH 1 using concentrated hydrochloric acid and then extracted with dichloromethane (2×50 ml). The extracts were dried (Na₂SO₄), filtered and concentrated in vacuo to provide the title compound as a colourless oil that crystallised over time. The aqueous phase was concentrated in vacuo to approximately half volume and the residual solid was separated from the supernatant and then stirred in methanol (5 ml) for 45 minutes. The suspension was filtered and the filtrate was mixed with the aqueous supernatent set apart earlier. This was left standing overnight. The resulting precipitate was filtered and washed (Methanol/water 1:1, 10 ml). The filtrate was concentrated in vacuo and the residue was slurried in methanol (20 ml) for 2 hours. The precipitate was filtered and washed with methanol (5 ml). The filtrate was concentrated in vacuo and methanol (10 ml) was added to the residue. The precipitate was filtered and washed with methanol (5 ml). The filtrate was concentrated in vacuo and the residue was combined with the two previous filtration residues. Partial purification by filtration through reverse phase silica (eluant: MeCN/water 4:1) was followed by filtration through Dowex 50XW8-200 ion exchange resin (˜80 ml) using (eluant: water). Concentration in vacuo provided a white powder which was slurried in iso-propanol (10 ml) at 50° C. for 2 hours before being filtered off to give the title compound as a white powder (357 mg, 34%). Microanalysis indicated 0.44% Cl present.

¹H NMR (400 MHz, DMSO-d₆) δ ppm 3.46 (9H, d, J 10 Hz), 3.18 (6H, d, J 22 Hz) and 2.34 (9H, s).

³¹P{¹H} NMR (161.97 MHz, DMSO-d₆) δ ppm 26.8.

¹³C NMR (100.6 MHz, DMSO-d₆) δ ppm 17.9, 28.9, 30.2, 51.4, 128.5 and 135.2.

### Example 16

### Preparation of [3,5-Bis-(Ethoxy-hydroxy-phosphorylmethyl)-2,4,6-trimethyl-benzyl]-phosphonic acid monoethyl ester (11)

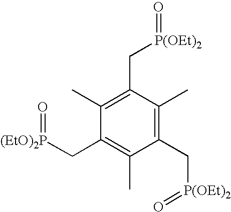

A solution of 2,4,6-tris methyl-1,3,5-tris benzyl bromide (1.00 g, 2.50 mmol) in triethylphosphite (5.0 mlo, 29.2 mmol) was heated under nitrogen at reflux for 19 hours. The reaction mixture was cooled to room temperature and concentrated in vacuo. The residue was purified by flash column chromatography (eluant: methanol/ethyl acetate 5:95 to 1:9 gradient) to provide the title compound as a colourless oil which slowly solidified upon standing at room temperature (1.05 g, 96%).

¹H NMR (400 MHz, CDCl₃) δ ppm 4.01-3.94 (12H, m), 3.34 (6H, d, J 22 Hz), 2.45 (9H, s) and 1.24 (18H, t, J 7 Hz).

³¹P{¹H} NMR (162 MHz, CDCl₃) δ ppm 28.5.

¹³C NMR (100.6 MHz, CDCl₃) δ ppm 16.4, 18.1, 28.8, 30.2, 61.8, 61.9, 127.7, 135.8 and 135.9.

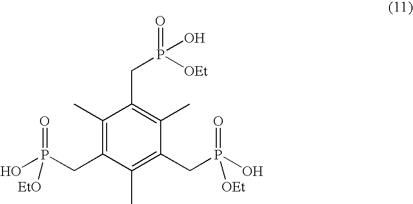

A solution of 2,4,6-tris methyl-1,3,5-tris benzyl diethyl phosphonate ester (92 mg, 0.16 mmol) and potassium hydroxide (105 mg, 1.87 mmol) in water (5 ml) was heated at reflux for 24 hours. Further potassium hydroxide (120 mg, 2.13 mmol) was added to the reaction mixture and heating at reflux was continued for a further 17 hours. The reaction mixture was cooled to room temperature, acidified to pH 1 using concentrated hydrochloric acid and extracted with dichloromethane (2×10 ml). The extracts were dried (MgSO₄), filtered and concentrated in vacuo to provide the title compound as a colorless oil that crystallized over time (5 lmg, 65% yield).

¹H NMR (400 MHz, CDCl₃) δ ppm 3.81 (3H, br s), 4.11(6H, q, J 7 Hz), 3.24 (6H, d, J 22 Hz), 2.39 (9H, s) and 1.35 (9H, t, J 7 Hz).

³¹P{¹H} NMR (162 MHz, CDCl₃) δ ppm 26.9

¹³C NMR (100.6 MHz, CDCl₃) δ ppm 15.4, 17.0, 27.3, 28.7, 60.2, 126.4 and 135.0.

### Example 17

### Preparation of [3,5-Bis-(Butoxy-hydroxy-phosphorylmethyl)-2,4,6-trimethyl-benzyl]-phosphonic acid monobutyl ester (12)

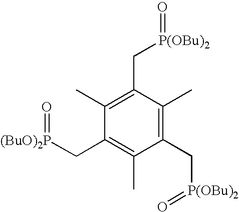

A solution of 2,4,6-tris methyl-1,3,5-tris benzyl bromide (1.03 g, 2.50 mmol) in tributylphosphite (5 ml, 18.2 mmol) was heated in an oil bath at 130° C. for 16.5 hours. The reaction mixture was cooled to room temperature, concentrated in vacuo and purified by flash column chromatography (eluant: ethyl acetate). The product obtained after concentration in vacuo was further purified by flash column chromatography (eluant: ethyl acetate/heptane 1:4 to 1:1 to ethyl acetate to methanol/ethyl acetate 5:95 gradient) to afford the title compound as a colorless oil (1.87 g, quantitative).

¹H NMR (400 MHz, CDCl₃) δ ppm 3.93-3.82 (12H, m), 3.32 (6H, d, J 22 Hz), 2.43 (9H, s), 1.59-1.52 (12H, m), 1.37-1.27 (12H, m) and 0.88 (18H, t, J 7 Hz).

³¹P{¹H} NMR (162 MHz, CDCl₃) δ ppm 28.7.

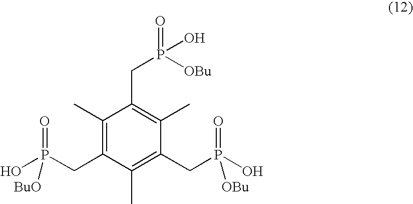

A solution of 2,4,6-tris methyl-1,3,5-tris benzyl di-n-butyl phosphonate ester (948 mg, 1.28 mmol) and potassium hydroxide (1.84 g, 32.7 mmol) in water (25 ml) and dioxane (20 ml) was heated at reflux for 24 hours. Further potassium hydroxide (0.99 g, 17.6 mmol) was added to the reaction mixture and heating at reflux was continued for 72 hours. The reaction mixture was cooled to room temperature, diluted with water (15 mL) and acidified to pH 1 using concentrated hydrochloric acid. The resultant suspension was filtered off and washed with water to give a white powder. The residue was slurred in dichloromethane (50 ml) for 3 hours and then filtered. The filtrate was concentrated in vacuo to give a yellow oil, analysis of which indicated incomplete conversion. The yellow oil was subsequently dissolved in dioxane (30 ml) and water (30 ml) and potassium hydroxide (2.61 g, 46.5 mmol) was added to the solution which was heated at reflux for 72 hours. The reaction mixture was cooled to room temperature then acidified to pH 1 using concentrated hydrochloric acid and extracted with dichloromethane (3×40 ml). The combined extracts were dried (Na₂SO₄), filtered and concentrated in vacuo to provide the title compound as a yellow foam (508 mg, 69%).

¹H NMR (400 MHz, DMSO-d₆) δ ppm 3.76 (6H, q, J 6 Hz), 3.13 (6H, d, J 22 Hz), 2.33 (9H, s), 1.52-1.45 (6H, m), 1.34-1.25 (6H, m) and 0.85 (9H, t, J 7 Hz).

³¹P{¹H} NMR (161.97 MHz, DMSO-d₆) δ ppm 25.3 and 28.4

¹³C NMR (100.6 MHz, DMSO-d₆) δ ppm 13.9, 17.9, 18.7, 29.4, 30.7, 32.5, 33.0, 64.1, 128.7 and 135.0.

### Example 18

### Preparation of (3,5-Bis-dimethylaminomethyl-benzyl)-dimethylamine (13)

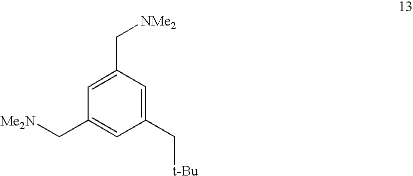

Dimethylamine was added via a balloon and needle to a stirred suspension of 1,3,5-tris-benzyl bromide (650 mg, 1.82 mmol), and potassium carbonate (1.18 g, 8.5 mmol) in toluene (10 ml). After 24 hours ¹H NMR analysis indicated incomplete conversion. Further potassium carbonate (449 mg, 3.24 mmol) and dimethylamine were added and stirring was continued for 24 hours. The reaction mixture was filtered through celite® and washed with toluene (10 ml). The filtrate was concentrated in vacuo to provide a mixture of solid and liquid (337 mg). ¹H NMR (CDCl₃) analysis indicated impure product, incomplete conversion had taken place. The residue was dissolved in toluene (15 mL) and potassium carbonate (970 mg, 7.0 mmol) and dimethylamine were added. The suspension was stirred at room temperature for 24 hours. Further potassium carbonate (306 mg, 2.21 mmol) and dimethylamine hydrochloride (231 mg, 2.83 mmol) were added followed by dimethylamine hours later. The resultant suspension was stirred at room temperature for 72 hours. The reaction mixture was filtered through celite® and washed with toluene (10 ml). The filtrate was concentrated in vacuo to provide the title compound as a yellow liquid (300 mg, 66%).

¹H NMR (400 MHz, CDCl₃) δ ppm 7.13 (3H, s), 3.39 (6H, s) and 2.22 (18H, s).

¹³C NMR (100.6 MHz, CDCl₃) δ ppm 45.4, 64.3, 128.6 and 138.8

### Example 19

### Preparation of (3,5-Bis-ethylaminomethyl-benzyl)-diethyl-amine (14)

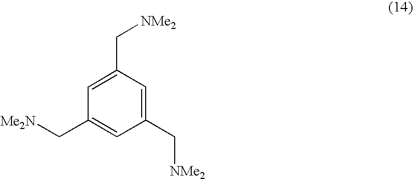

A suspension of 1,3,5-tris-benzyl bromide (441 mg, 1.23 mmol), potassium carbonate (821 mg, 5.94 mmol) and diethylamine (1.30 ml, 12.4 mmol) in toluene (10 ml) was stirred at room temperature for 72 hours under a nitrogen atmosphere. The reaction mixture was filtered through celite® and washed with toluene (10 ml). The filtrate was concentrated in vacuo to provide the title compound as a yellow oil (397 mg, 97%).

¹H NMR (400 MHz, CDCl₃) δ ppm 7.15 (3H, s), 3.54 (6H, s), 2.51 (12H, q, J 7 Hz) and 1.03 (18H, t, J 7 Hz).

¹³C NMR (100.6 MHz, CDCl₃) δ ppm 11.7, 46.7, 57.4, 128.2 and 139.3.

### Example 20

### Preparation of (3,5-Bis-dibutylaminomethyl-benzyl)-dibutyl-amine (15)

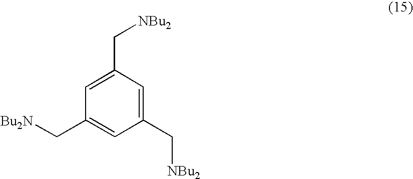

A suspension of 1,3,5-tris-benzyl bromide (303 mg, 0.84 mmol), potassium carbonate (60 mg, 4.3 mmol) and dibutylamine (1.2 ml, 7.1 mmol) in toluene (10 ml) was stirred at room temperature for 18.5 hours under a nitrogen atmosphere. The reaction mixture was filtered through celite® and washed with toluene (10 ml). The filtrate was concentrated in vacuo to provide the title compound as a colorless oil (402 mg, 95%).

¹H NMR (400 MHz, CDCl₃) δ ppm 7.13 (3H, s), 3.52 (6H, s), 2.41-2.37 (12H, m), 1.48-1.41 (12H, m), 1.31-1.22 (12H, m) and 0.86 (18H, t, J 7 Hz).

¹³C NMR (100.6 MHz, CDCl₃) δ ppm 14.1, 20.6, 29.3, 53.5, 58.5, 128.0 and 139.5.

### Example 21

### Preparation of (3,5-Bis-dimethylaminomethyl-2,4,6-trimethyl-benzyl)-dimethyl-amine (16)

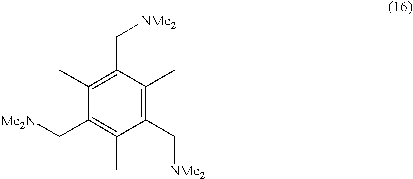

Dimethylamine was added via a balloon and a needle to a suspension of 2,4,6-methyl-1,3,5-tris-benzyl bromide (901 mg, 2.25 mmol) and potassium carbonate (1.58 g, 11.4 mmol) in toluene (20 ml). The suspension was stirred at room temperature for one day and was then filtered through celite® and washed with toluene (10 ml). The filtrate was concentrated in vacuo to provide the title compound as a white solid (518 mg, 79%).

¹H NMR (400 MHz, CDCl₃) δ ppm 3.46 (6H, s), 2.42 (9H, s) and 2.28 (18H, s).

¹³C NMR (100.6 MHz, CDCl₃) δ ppm 16.3, 45.0, 57.6, 133.4 and 137.1.

### Example 22

### Preparation of (3,5-Bis-diethylaminomethyl-2,4,6-trimethyl-benzyl)-diethyl-amine (17)

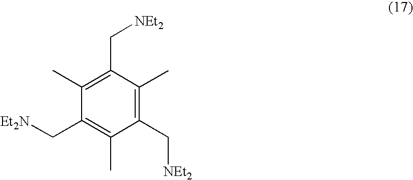

A suspension of 2,4,6-methyl-1,3,5-tris-benzyl bromide (1.00 g, 2.50 mmol), potassium carbonate (1.73 g, 12.5 mmol) and diethylamine (2.60 ml, 25.0 mmol) in toluene (10 ml) was heated in an oil bath at 75° C. for 72 hours under a nitrogen atmosphere. The mixture was then cooled to room temperature, filtered through celite® and washed with toluene (10 ml). The filtrate was concentrated in vacuo to provide the title compound as a light yellow oil which slowly solidified upon standing at room temperature (890 mg, 94%).

¹H NMR (400 MHz, CDCl₃) δ ppm 3.59 (6H, s), 2.48-2.44 (21H, m) and 0.98 (18H, t, J 8 Hz).

¹³C NMR (100.6 MHz, CDCl₃) δ ppm 12.6, 17.0, 46.5, 53.0, 134.0 and 137.6.

### Example 23

### Preparation of (3,5-Bis-dibutylaminomethyl-2,4,6-trimethyl-benzyl)-dibutyl-amine (18)

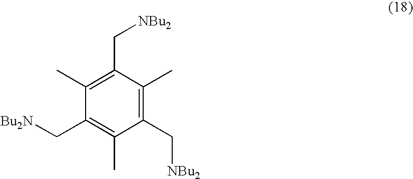

A suspension of 2,4,6-methyl-1,3,5-tris-benzyl bromide (1.17 g, 2.9 mmol), potassium carbonate (2.05 g, 14.8 mmol) and dibutylamine (5.0 ml, 29.6 mmol) in toluene (20 ml) was heated in an oil bath at 75° C. for 72 hours under a nitrogen atmosphere. The mixture was cooled to room temperature, dried (Na₂SO₄), filtered through celite® and washed with toluene (10 ml). The filtrate was concentrated in vacuo to provide the title compound as a light yellow solid (1.44 g, 89%).

¹H NMR (400 MHz, CDCl₃) δ ppm 3.57 (6H, s), 2.42 (9H, s), 2.34 (12H, t, J 7 Hz), 1.38 (12H, m), 1.19 (12H, m) and 0.81 (18H, t, J 8 Hz). ¹³C NMR (100.6 MHz, CDCl₃) δ ppm 14.1, 16.5, 20.7, 29.4, 52.8, 53.5, 133.4 and 137.3.

### Example 24

### Preparation of 2,2,2-Trifluoro-N-{3-[3-(2,2,2-trifluoro-acetylamino)-propylamino]-propyl}-acetamide

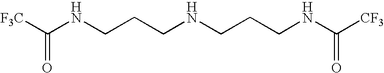

Ethyl trifluoroacetate (3.6 ml, 30.5 mmol) was added in a dropwise manner over 10 minutes to a stirred solution of bis(3-aminopropyl)amine (2.1 ml, 13.2 mmol) in methanol (20 ml) at −78° C. under a nitrogen atmosphere. After one hour the mixture was placed in an ice-water bath and after a further hour was placed in a freezer (external temperature: −20° C.) for 14 hours. The solution was concentrated in vacuo to provide the title compound as a colourless oil which was used without further purification (4.09 g, 83%).

¹H NMR (400 MHz, CDCl₃) δ ppm 8.51 (2H, br), 3.45 (4H, br t, J 7 Hz), 2.74 (4H, t, J 7 Hz), 1.74 (4H, quintet, J 7 Hz) and 1.48-1.31 (1H, br s).

¹³C NMR (100.6 MHz, CDCl₃) δ ppm 28.1, 39.6, 48.2, 116.1 (q, J 288 Hz) and 157.7 (q, J 37 Hz).

¹⁹F{¹H} NMR (376.5 MHz, CDCl₃) δ ppm −76.6.

### Example 25

### Preparation of [(Bis-{3-[(hydroxy-methoxy-phosphorylmethyl)-amino]-propyl}-amino)-methyl]-phosphonic acid monomethyl ester (19)

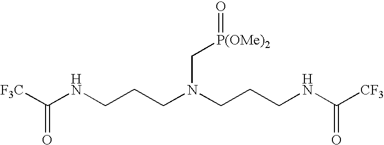

A solution of formaldehyde (1.19 g of a 37 wt % solution in water, 14.65 mmol) in methanol (5 ml) was added to a stirred solution of (50) (1.43 g, 4.44 mmol) and trimethylphosphite (1.7 ml, 14.65 mmol) in methanol (5 ml) under a nitrogen atmosphere. After 24 hours the mixture was concentrated in vacuo and the residue was purified by flash column chromatography (eluent: ethyl acetate/heptane 9:1) to provide the title compound as a colourless oil (1.82 g, 92%).

¹H NMR (400 MHz, CDCl₃) δ ppm 7.90 (2H, br s), 3.79 (3H, s), 3.77 (3H, s), 3.48-3.43 (4H m), 2.82 (2H, d, J 11 Hz), 2.62 (4H, t, J 6 Hz) and 1.69 (4H, quintet, J 6 Hz).

¹⁹F{¹H} NMR (376.5 MHz, CDCl₃) δ ppm −76.3

³¹P{¹H} NMR (400.1 MHz, CDCl₃) δ ppm 28.8.

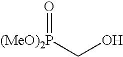

A stirred mixture of dimethylphosphite (10.0 ml, 109 mmol), triethylamine (1.5 ml, 10.9 mmol) and paraformaldehyde (3.26 g) was heated in an oil bath at 100° C. (external temperature). After 2.5 hours the mixture was cooled to room temperature, concentrated in vacuo and the residue was purified by flash column chromatography (eluent: ethyl acetate/methanol 95:5) to provide the title compound as a colourless oil (9.41 g, 62%).

¹H NMR (400 MHz, CDCl₃) δ ppm 4.32 (1H, br s), 3.95 (2H, d, J 6 Hz), 3.83 (3H, s) and 3.81 (3H, s).

³¹P{¹H} NMR (400.1 MHz, CDCl₃) δ ppm 27.8.

¹³C NMR (100.6 MHz, CDCl₃) δ ppm 53.5, 56.0 and 57.6.

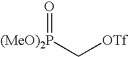

Trifluoromethanesulfonic anhydride (4.7 ml, 27.8 mmol) was added in a dropwise manner to a stirred solution of dimethy phosphonic ester alcohol (3.54 g, 25.3 mmol) and 2,6-lutidene (3.5 ml, 30.3 mmol) in dichloromethane (30 ml) ensuring that the internal temperature remained below −50° C. Once the addition was complete the mixture was slowly warmed to 0° C. over approximately 90 minutes. Diethyl ether (150 ml) was then added and the resultant suspension was filtered through celite® and washed with diethyl ether (20 ml). The solution was then sequentially washed with water (90 ml), 1M hydrochloric acid (90 ml) and brine (90 ml). After drying (Na₂SO₄), filtration followed by concentration provided the title compound as a light yellow oil (5.67 g, 83%) which was used without further purification.

¹H NMR (400 MHz, CDCl₃) δ ppm 4.87 (2H, d, J 9 Hz), 3.91 (3H, s) and 3.89 (3H, s)

¹⁹F{¹H} NMR (376.5 MHz, CDCl₃) δ ppm −74.5

³¹P{¹H} NMR (400.1 MHz, CDCl₃) δ ppm 16.1

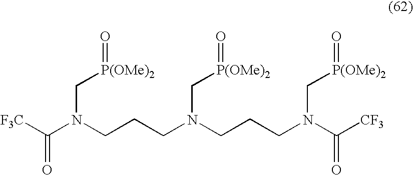

A solution of spermidine bis-trifluoroamide derivative (1.80 g, 4.05 mmol) in DMF (6 ml) was added in a dropwise manner to a stirred ice-water bath cooled suspension of sodium hydride (340 mg of a 60% suspension in mineral oil, 8.51 mmol) in DMF (6 ml). After stirring the resultant solution at this temperature for one hour, a solution of diemthy phosphonate triflate derivative (3.09 g, 11.4 mmol) in DMF (6 ml) was added in a dropwise manner. The resultant solution was slowly warmed to room temperature and stirred for a further 72 hours. The mixture was then diluted with MTBE (60 ml) and saturated ammonium chloride solution (15 ml) was added. The layers were separated and the organic phase was washed with water (4×15 ml) and the combined aqueous washes were then re-extracted with ethyl acetate (2×15 ml). The combined extracts were washed with brine (30 ml), dried (Na₂SO₄), filtered and concentrated in vacuo. Purification by flash column chromatography (eluent: ethyl acetate to methanol/ethyl acetate 1:5 gradient) provided the title compound as a light yellow oil (651 mg, 23%).

¹H NMR (400 MHz, CDCl₃) δ ppm 3.92 (0.8H, d, J 12 Hz), 3.90 (3.2H, d, J 12 Hz), 3.82 (1H, s), 3.81 (4.75H, s), 3.80 (1H, s), 3.79 (3.25H, s), 3.78 (4.75H, s), 3.76 (3.25H, s), 3.64 (4H, t, J 7 Hz), 2.87 (0.3H, d, J 10 Hz), 2.85 (1.7H, d, J 10 Hz), 2.62 (4H, t, J 7 Hz) and 1.85-1.79 (4H, m).

¹⁹F{¹H} NMR (376.5 MHz, CDCl₃) δ ppm −67.4 and −69.2.

³¹P{¹H} NMR (400.1 MHz, CDCl₃) δ ppm 28.6, 24.2 and 22.6.

({{3-[(Dimethoxy-phosphorylmethyl)-(2,2,2-trifluoro-acetyl)-amino]-propyl}-[3-(2,2,2-trifluoro-acetylamino)-propyl]-amino}-methyl)-phosphonic acid dimethyl ester (64) was also isolated (860 mg, 38%).

¹H NMR (400 MHz, CDCl₃) δ ppm 8.80 (1H, br s), 3.87 (2H, d, J 12 Hz), 3.80 (3H, s), 3.79 (3H, s), 3.78 (3H, s), 3.77 (3H, s), 3.66-3.60 (2H, m), 3.51-3.46 (2H, m), 2.83 (0.4H, d, J 11 Hz), 2.82 (1.6H, d, J 11 Hz), 2.69-2.64 (2H, m), 2.52 (2H, t, J 7 Hz), 1.79 (2H, quintet, J 7 Hz) and 1.69-1.64 (2H, m).

¹⁹F{¹H} NMR (376.5 MHz, CDCl₃) δ ppm −68.9, −69.3 and −76.2

³¹P{¹H} NMR (400.1 MHz, CDCl₃) δ ppm 28.9, 23.8 and 22.1.

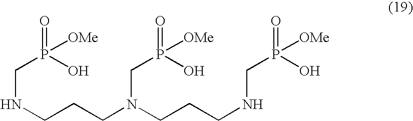

Potassium hydroxide (7.7 ml of a 10% w/v solution in water, 13.7 mmol) was added to a stirred solution of the amide (473 mg, 0.69 mmol) in dioxane (3.8 ml). The mixture was then heated, under nitrogen, in an oil bath at 105° C. After 17 hours the mixture was cooled to room temperature, placed in an ice-water bath and acidified to pH 0.4 with 6M hydrochloric acid. The resultant mixture was concentrated in vacuo. Toluene (6 ml) was added to the residue and the resultant suspension was concentrated in vacuo. This process was repeated four times. Dioxane (2 ml) and water (2 ml) were than added to the residue which was stirred until dissolution was complete. Potassium hydroxide (10% w/v solution in water) was then added dropwise to take the solution to pH 7.0. The solution was then concentrated in vacuo. Toluene (6 ml) was added to the residue and the resultant suspension was concentrated in vacuo. This process was repeated four times. Methanol (10 ml) was added to the residue and the resultant suspension was stirred at room temperature under nitrogen. After 17 hours the suspension was filtered through celite® and washed with fresh methanol (10 ml). The filtrate was concentrated in vacuo and the residue was purified by reverse-phase HPLC to provide the title compound as an off-white solid (138 mg, 44%) of approximately 80% purity.

¹H NMR (400 MHz, CD₃OD) δ ppm 3.56 (3H, s), 3.53 (3H, s), 3.51 (1.5H, s), 3.50 (1.5H, s), 3.22-3.20 (4H, m), 2.99 (3H, d, J 12 Hz), 2.92 (1H, d, J 12 Hz), 2.61-2.51 (6H, m) and 1.83-1.73 (4H, m).

³¹P{¹H} NMR (400.1 MHz, CD₃0D) δ ppm 22.2 and 12.9.

### Example 26

### Preparation of [(Bis-{3-[(ethoxy-hydroxy-phosphorylmethyl)-amino]-propyl}-amino)-methyl]-phosphonic acid monoethyl ester (20)

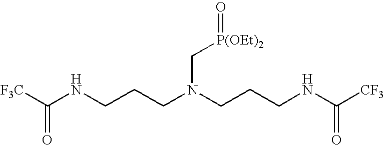

Using the procedure described for 19 above, the amide (1.00 g, 3.10 mmol) was treated with triethylphosphite (1.8 ml, 10.2 mmol) and formaldehyde (831 mg of a 37 wt % solution in water, 10.2 mmol) in ethanol (10 ml). Purification by flash column chromatography (eluent: ethyl acetate/heptane 9:1) provided the title compound as a colourless oil (1.34 g, 91%).

¹H NMR (400 MHz, CDCl₃) δ ppm 7.88 (2H, br s), 4.17-4.09 (4H, m), 3.46 (4H, q, J 7 Hz), 2.79 (2H, d, J 11 Hz), 2.61 (4H, t, J 7 Hz), 1.69 (4H, quintet, J 7 Hz) and 1.34 (6H, t, J 7 Hz).

¹⁹F{¹H} NMR (376.5 MHz, CDCl₃) δ ppm −76.3.

³¹P{¹H} NMR (400.1 MHz, CDCl₃) δ ppm 26.5

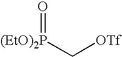

Using the procedure for described above, the alcohol (3.42 g, 20.3 numol) was treated with 2,6-lutidene (2.8 ml, 24.4 mmol) and trifluoromethanesulfonic anhydride (3.8 ml, 22.4 mmol) in dichloromethane (30 ml) to provide the title compound as a light yellow oil (4.29 g, 70%). This was used without further purification.

¹H NMR (400 MHz, CDCl₃) δ ppm 4.62 (2H, d, J 9 Hz), 4.29-4.22 (4H, m) and 1.38 (6H, t, J 7 Hz).

¹⁹F{¹H} NMR (376.5 MHz, CDCl₃) δ ppm −74.4.

³¹P{¹H} NMR (400.1 MHz, CDCI₃) δ ppm 13.5.

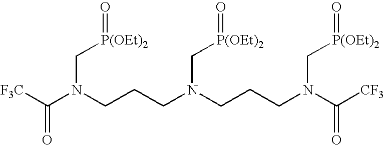

Using the procedure described for 19 above, the bis-amide (1.32 g, 2.79 mmol) was treated with sodium hydride (230 mg of a 60% dispersion in mineral oil, 5.86 mmol) and the diethyl phosophante triflate (4.84 g, 16.2 mmol) in DMF (20 ml). Following isolation of the crude reaction products, purification by flash column chromatography (eluent: ethyl acetate/heptane 4:1 to ethyl acetate to methanol/ethyl acetate 5:95 gradient) provided the title compound as a light yellow oil (473 mg, 22%) of approximately 75% purity. Further purification by reverse-phase HPLC provided the title compound as a colourless oil.

¹H NMR (400 MHz, CDCl₃) δ ppm 4.17-4.09 (12H, m), 3.89 (2.9H, d, J 11 Hz), 3.82 (1.1H, d, J 11 Hz), 3.67-3.61 (4H, br m), 2.87 (0.5H, d, J 11 Hz), 2.84 (1.5H, d, J 11 Hz), 2.68-2.62 (4H, br m), 1.85-1.78 (4H, br m) and 1.48-1.39 (18H, m).

¹⁹F{¹H} NMR (376.5 MHz, CDCl₃) δ ppm −68.7 and −69.2.

³¹P{¹H} NMR (400.1 MHz, CDCl₃) δ ppm 26.3, 21.5 and 19.8.

({{3-[(Diethoxy-phosphorylmethyl)-(2,2,2-trifluoro-acetyl)-amino]-propyl}-[3-(2,2,2-trifluoro-acetylamino)-propyl]-amino}-methyl)-phosphonic acid diethyl ester (65) was also isolated (574 mg, 33%).

¹H NMR (400 MHz, CDCl₃) δ ppm 8.99 (1H, br t), 4.18-4.09 (8H, m), 3.87 (1.6H, d, J 12 Hz), 3.79 (0.4H, d, J 12 Hz), 3.65-3.61 (2H, m), 3.50-3.46 (2H, m), 2.81 (0.66H, d, J 12 Hz), 2.80 (1.33H, d, J 12 Hz), 2.69-2.66 (2H, m), 2.52 (2H, t, J 7 Hz), 1.82-1.78 (2H, m), 1.68-1.64 (2H, m) and 1.35-1.31 (12H, m).

¹⁹F{¹H} NMR (376.5 MHz, CDCl₃) δ ppm −68.7, −69.3 and −76.1.

³¹P{¹H} NMR (400.1 MHz, CDCl₃) δ ppm 26.5, 21.1 and 19.4

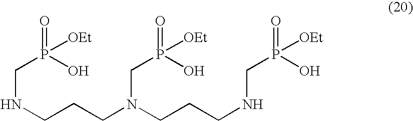

Using the procedure described for (19) above, a solution of the amide (103 mg, 0.13 mmol) in dioxane (1.5 ml) was treated with potassium hydroxide (1.5 ml of a 10% w/v solution in water, 2.66 mmol). Following the acidification and neutralisation steps, iso-propanol (6 ml) was used to extract the product. Filtration through celite®, washing with fresh iso-propanol (10 ml) and concentration in vacuo provided the title compound as a light yellow solid (53 mg, 82%) of approximately 90% purity which was not purified further.

¹H NMR (400 MHz, CD₃OD) δ ppm 3.91-3.84 (4H, m), 3.14 (4H, t, J 7 Hz), 2.98 (4H, d, J 12 Hz), 2.66-2.61 (4H, m), 2.56 (2H, d, J 12 Hz), 1.82-1.79 (4H, m) and 1.20 (6H, t, J 7 Hz).

³¹P{¹H} NMR (400.1 MHz, CD₃OD) δ ppm 20.0 and 10.7.

### Example 27

### Preparation of {[Bis-(3-methylamino-propyl)-aminol-methyl}-phosphonic acid monobutyl ester

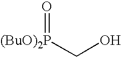

A stirred mixture of dibutylphosphite (10.0 ml, 51.5 mmol), triethylamine (720 μl, 5.15 mmol) and paraformaldehyde (1.54 g) was heated in an oil bath at 100° C. (external temperature). After 6 hours the mixture was cooled to room temperature, concentrated in vacuo and the residue was purified by flash column chromatography (eluent: ethyl acetate/heptane 9:1 to ethyl acetate gradient) to provide the title compound as a colourless oil (2.53 g, 22%).

¹H NMR (400 MHz, CDCl₃) δ ppm 4.14-4.08 (4H, m), 3.90 (2H, br t), 3.42 (1H, br s), 1.70-1.63 (4H, m), 1.41 (4H, sextet, J 7 Hz) and 0.94 (6H, t, J 7 Hz).

³¹P{¹H} NMR (400.1 MHz, CDCl₃) δ ppm 25.1.

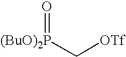

Using the procedure for preparing 19 described above, the alcohol (2.52 g, 11.3 mmol) was treated with 2,6-lutidene (1.6 ml, 13.5 mmol) and trifluoromethanesulfonic anhydride (2.1 ml, 12.4 mmol) in dichloromethane (15 ml) to provide the title compound as a colourless oil (3.66 g, 91%). This was used without further purification.

¹H NMR (400 MHz, CDCl₃) δ ppm 4.62 (2H, d, J 9 Hz), 4.18 (4H, q, J 7 Hz), 1.70 (4H, quintet, J 7 Hz) and 1.42 (6H, sextet, J 7 Hz).

¹⁹F{¹H} NMR (376.5 MHz, CDCl₃) δ ppm −74.4.

³¹P{¹H} NMR (400.1 MHz, CDCl₃) δ ppm 13.5.

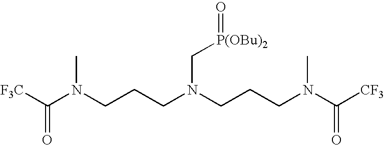

A solution of the bis-amide (366 mg, 0.69 mmol) in DMF (1 ml) was added under nitrogen in a dropwise manner to an ice-water bath cooled stirred suspension of sodium hydride (61 mg of a 60% dispersion in mineral oil, 1.52 mmol) in DMF (1 ml). After 2 hours stirring at this temperature, methyl iodide (111 μl, 1.79 mmol) was added dropwise and the resultant mixture was stirred at room temperature. After 24 hours, the mixture was diluted with MTBE (20 ml) and saturated ammonium chloride solution (10 ml) was added. The layers were separated and the organic phase was washed with water (5×10 ml). The combined aqueous washes were then extracted with MTBE (2×10 ml) and the combined organic extracts were washed with brine (20 ml), dried (MgSO₄), filtered and concentrated in vacuo. The crude material was purified by flash column chromatography (eluent: ethyl acetate/heptane 3:1) to provide the title compound as a colourless oil (220 mg, 57%) of approximately 70% purity, as determined by inspection of the associated ¹H NMR spectra.

¹H NMR (400 MHz, CDCl₃) δ ppm 4.05 (4H, q, J 7 Hz), 3.45-3.44 (4H, m), 3.14 (4H, s), 3.04 (2H, s), 2.87 (1.33H, d, J 12 Hz), 2.81 (0.66H, d, J 12 Hz), 2.67-2.60 (4H, m), 1.79-1.72 (4H, m), 1.63 (4H, quintet, J 7 Hz), 1.43-1.35 (4H, m) and 0.94 (6H, t, J 7 Hz).

¹⁹F{¹H} NMR (376.5 MHz, CDCl₃) δ ppm −69.2 and −70.3.

³¹P{¹H} NMR (400.1 MHz, CDCl₃) δ ppm 26.7.

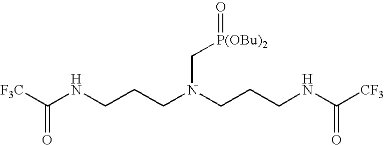

Using the procedure described for 19 above, the amide (1.59 g, 4.93 mmol) was treated with tributylphosphite (4.4 ml, 16.3 mmol) and formaldehyde (1.32 g of a 37 wt % solution in water, 16.3 mmol) in butanol (10 ml). Purification by flash column chromatography (eluent: ethyl acetate/heptane 1:1) provided the title compound as a colourless oil (2.45 g, 94%) of approximately 75% purity.

¹H NMR (400 MHz, CDCl₃) δ ppm 7.88 (2H, br s), 4.09-4.02 (4H, m), 3.48-3.43 (4H, m), 2.79 (2H, d, J 11 Hz), 2.61 (4H, t, J 6 Hz), 1.70-1.68 (8H, m), 1.45-1.37 (4H, m) and 0.96-0.92 (6H, m).

¹⁹F{¹H} NMR (376.5 MHz, CDCl₃) δ ppm −76.3.

³¹P{¹H} NMR (400.1 MHz, CDCl₃) δ ppm 26.4.

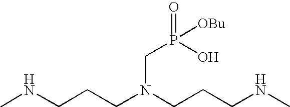

Using the procedure described for (19) above, a solution of the amide (217 mg, 0.39 mmol) in dioxane (4.4 ml) was treated with potassium hydroxide (4.4 ml of a 10% w/v solution in water, 7.80 mmol). Following the acidification and neutralisation steps, iso-propanol (6 ml) was used to extract the product. Filtration through celite®, washing with fresh iso-propanol (6 ml) and concentration in vacuo provided the title compound as a yellow oil (62 mg, 52%) of approximately 75% purity which was not purified further.

¹H NMR (400 MHz, CDCl₃) δ ppm 10.12 (2H, br s), 3.88 (2H, q, J 6 Hz), 3.13-3.03 (4H, m), 2.72-2.69 (4H, m), 2.62 (6H, s), 1.99-1.96 (4H, m), 1.62-1.58 (2H, m), 1.38 (2H, sextet, J 7 Hz) and 0.92 (3H, t, J 7 Hz).

³¹P{¹H} NMR (400.1 MHz, CDCl₃) δ ppm 21.1.

### Example 28

### Preparation of N-(3-Diethylamino-propyl)-N,N′,N′-triethyl-propane-1,3-diamine (23)

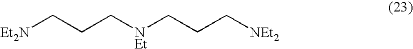

Bromoethane (37.0 ml, 500 mmol) was added in a dropwise manner to a stirred suspension of bis(3-aminopropyl)amine (10.0 ml, 71.5 mmol) and potassium carbonate (49.7 g, 357 mmol) in ethanol (150 ml) under a nitrogen atmosphere. The mixture was then heated to 45° C. After 6 days, the mixture was cooled to room temperature, filtered through celite® and washed with ethanol (20 ml). The filtrate was concentrated in vacuo to afford a yellow oil which slowly solidified upon standing. The crude product was suspended in toluene (100 ml) and was stirred with 2M sodium hydroxide (100 ml) for 20 minutes. The layers were separated and the organic phase was concentrated in vacuo. The residue obtained was dissolved in toluene (100 ml) and concentrated in vacuo to provide the title compound of approximately 90% purity (1.52 g, 8%). A portion was purified by Kügelrohr distillation (81° C., 0.032 mbar) to provide the title compound as a colourless oil.

¹H NMR (400 MHz, CDCl₃) δ ppm 2.52 (10H, q, J 7 Hz), 2.44-2.40 (8H, m), 1.64-1.56 (4H, m) and 1.04-0.99 (15H, m).

¹³C NMR (100.6 MHz, CDCl₃) δ ppm 11.7,24.4, 46.9,47.4, 51.1 and 51.7.

### Example 29

### Permeability of Tb-PCTMB in In Vitro Cancer Cells

Tb-PCTMB (Tb(III) 3,6,9-tris(methylene phosphonic acid n-butyl ester)-3,6,9,15-tetraazabicyclo[9.3.1]pentadeca-1(15),11,13-triene) was tested for permeability to abnormal and disease-state cells. Permeability of Tb-PCTMB to epithelial cancer cell lines, LNCaP, T84, Caco-2, and RBL was measured through detection of the intensity of its inherent fluorescence (excitation wavelength of 270 nm, emission at 540 nm).

LNCaP cell line is a human prostate carcinoma. Cells were grown in RPMI 1640 Medium supplemented with 10% FBS, 2 mM L-glutamine, glucose (2 g/L) augmented with gentamicin (50 μg/mL), penicillin/streptomycin (100 IU/ml/100 μg/mL) and amphotericin B (2.5 μg/mL). Caco-2 cell line is a human colorectal carcinoma. These cells were grown in Eagle's Minimum Essential Medium supplemented with 20% FBS, Earle's BSS, 2 mM L-glutamine, 1.0 mM sodium pyruvate, 0.1 mM nonessential amino acids augmented with gentamicin (50 μg/mL), penicillin/streptomycin (100 IU/mL, 100 μg/mL), and amphotericin B (2.5 μg/mL). T-84 cell line is a human colorectal carcinoma. Cells were grown in 1:1 mixture of Ham's F12 medium and Dulbecco's Modified Eagle's Medium supplemented with 5% FBS augmented with 50 μg/ml gentamicin (50 μg/mL), penicillin/streptomycin (100 IU/mL, 100 μg/mL), and amphotericin B (2.5 μg/mL).

RBL-2H3 is a rat basophilic leukemia cell line. These cells were grown in Iscove's Medium supplemented with 2 mM L-glutamnine, penicillin/streptomycin (100 IU/mL, 100 μg/mL) and 10% FBS. Cells were maintained at 5% CO₂ and 95% humidity. PZ-HPV-7 cell line was derived from epithelial cells cultured from normal prostate tissue. They were grown in Keratinocyte-Serum Free Medium with human recombinant EGF and bovine pituitary extract supplemented with 50 μg/mL gentamicin and penicillin/streptomycin (100 IU/mL, 100 μg/mL). All media and supplements were purchased from Gibco BRL (Grand Island, N.Y., USA). All cell lines were obtained from ATCC and were maintained at 37° C., 5% CO₂ and 95% humidity.

Cells were grown to confluence in T-150 flasks (about 2×10⁶ cells). Before treating with Tb-PCTMB, cells were rinsed twice with warm 10 mM HEPES (Gibco BRL, Grand Island N.Y.), and then treated with either 5 mL of 0, 1, 500, 1000, or 2000 μM Tb-PCTMB in 10 mM HEPES, pH 7.4, 5 mM KCl, 150 mM NaCl, 0.7 mM NaH₂PO₄ for 2 hours at 37° C. (dose response study) or with 5 mL of Tb-PCTMB (1 mM final concentration) for the following duration: 0, 1, 2, 4, and 8 hours (time course study). After treatment, the Tb-PCTMB supernatant was discarded and the cells were rinsed three times at room temperature with 10 mM HEPES. Cells were then removed from the flask by manual scraping. Cells were transferred to a 50-mL conical tube and centrifuged (400×g) for 5 minutes and the test material removed. The resulting cell pellet was isolated for cytoplasmic and membrane fractions and was analyzed by measuring the inherent fluorescence of the Tb-PCTMB compound.

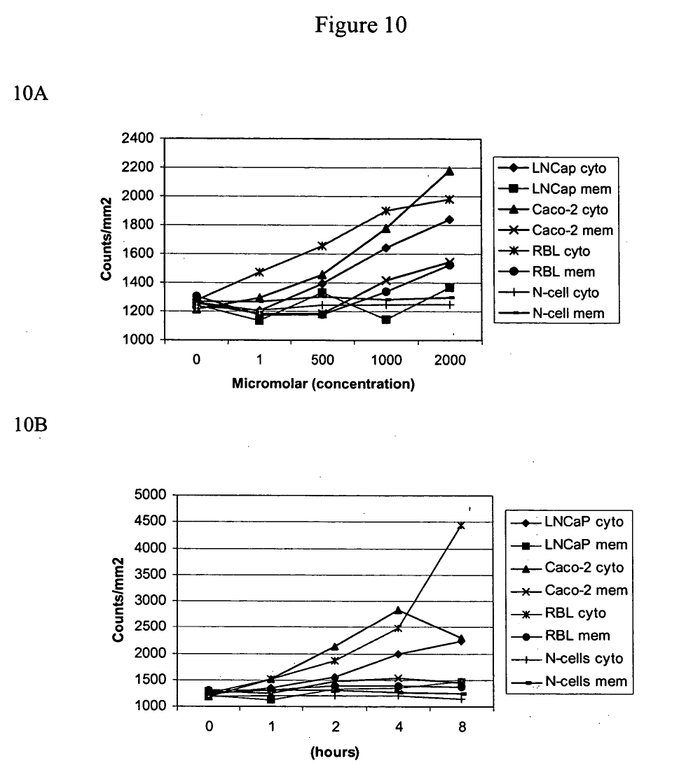

Dose response curves and time course curves for each cell line were generated, (FIGS. 10A and 10B). Carcinoma cell lines, LNCaP, Caco-2, and RBL-2H3, exhibited significant increases in fluorescence in the cytoplasm as the concentration of increased Tb-PCTMB compared to a normal cell line, PZ-HPV-7. In the membrane fractions, Caco-2 and RBL-2H3 have detectable quantity of fluorescence and show an increase in fluorescence as the concentration increases compared to the normal cell line, PZ-HPV-7.

Tb-PCTMB therefore is permeable to cancer cell membranes with the majority of the fluorescence associated with the cytoplasm versus the membrane fractions. More importantly, Tb-PCTMB is significantly less detectable in normal cells, further supporting the specificity to cancer cells over normal cell lines.

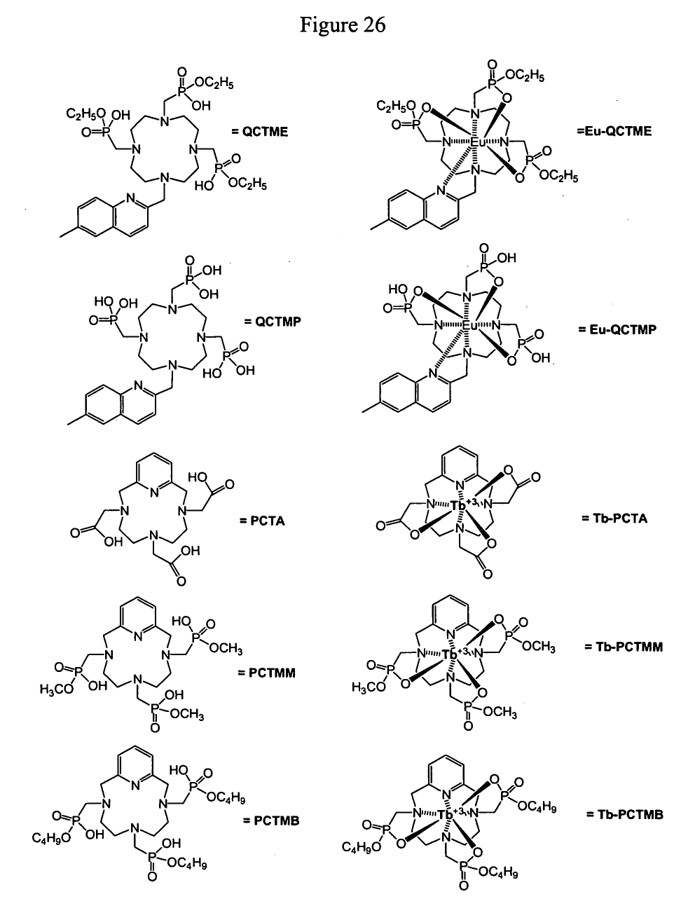

Furthermore, two analogues (PCTMM and PCTA, as shown in FIG. 26) with decreasing lipophilic character were evaluated. To explore the effect of reducing the lipophilic character, LNCaP, Caco-2, T84, and PZ-HPV-7 were analyzed to determine the dose response to Tb-PCTMB, Tb-PCTMM, and Tb-PCTA. In this study, the cytoplasm and membrane fractions were extracted by Mem-PER Eukaryotic Membrane Protein Extraction Reagent Kit as described by the manufacturers (Pierce Biotechnology, Rockford, Ill.) and analyzed by a FLUOR-S MULTIIMAGER. A dose response to each cell line was generated, where the cells were incubated for 2 h with 1 mM of Tb-PCTMB, Tb-PCTMM, or Tb-PCTA (Table 1).

TABLE 1
Cancer Cell Dose Responses

| | |
|---|---|
| Cancer | Fluorescence/mg |

| Cells | Chelate | Cytoplasm | Membrane |
|---|---|---|---|
| LNCaP | Tb-PCTMB | 616.8 | 448.9 |
| | Tb-PCTMM | 344.5 | 379.5 |
| | Tb-PCTA | 368.8 | 362.1 |
| T84 | Tb-PCTMB | 476.4 | 355.5 |
| | Tb-PCTMM | 328.3 | 358.7 |
| | Tb-PCTA | 333.4 | 329.1 |
| Caco-2 | Tb-PCTMB | 481.6 | 398.6 |
| | Tb-PCTMM | 357.7 | 388.9 |
| | Tb-PCTA | 366.3 | 362.8 |

Fluorescence was significantly greater in the cytoplasm of LNCaP, Caco-2, and T84 cancer cells in the presence of Tb-PCTMB (this was not due to any difference in innate fluorescence among the chelates tested). More importantly, compared to results seen in normal cells (non-cancerous control cells), these tests detected no permeability of either Tb-PCTMM or Tb-PCTA to cell membranes, as no increased fluorescence thereof was detectable in the cytoplasm. Additionally, the less lipophilic cognate chelate structures, Tb-PCTMM and Tb-PCTA, did not show any specificity or selectivity for cancer cells versus normal cells. Collectively, these data verify that the lipophilic nature of Tb-PCTMB is important in the chelate's ability to cross the membrane into the cytoplasm. Thus, the key physico-chemical features of chelates for disease-specific intracellular uptake (lipophilicity, phosphonate group architecture) are well illustrated by this data.

### Example 30

### Localization of Tb-PCTMB in the Cytoplasm of Cancer Cells

Tb-PCTMB was administered in vitro to both a healthy, control cell line (PZ-HPV-7 cell line), and to LNCaP, T84, Caco-2, and RBL-2H3 cancer cell lines, for 2 hours. Cell samples were lysed in parallel by sonication or by lysis in hypotonic buffer. Cell fractions were collected from discontinuous sucrose gradients and from separate Percoll gradients, after ultracentrifugation.

Cell fractions exhibiting fluorescence were further fractionated and enriched by using 0.5-mL Eppendorf Centrifugal Filter Tubes (30 kDa molecular weight cut-off). Following fractionation, each sample was lyophilized to enrich the sample. The enriched samples were analyzed by high resolution SDS-PAGE, thus identifying one major fluorescent band, with an apparent molecular weight of 15 kDa; this was confirmed by Coomassie staining. A comparison of Coomassie/Fluorescence SDS-PAGE gel patterns of proteins isolated from the different cell lines (Caco-2, T84, and RBL-2H3) resulted in the identification of a similar Tb-PCTMB:protein complex at about 15 kDa.

### Example 31

### Characterization of Tb-PCTMB Binding Partner Biomolecule

The protein:Tb-PCTMB complex was further analyzed to identify the protein by excising the corresponding (about 15 kDa) protein band from the gel, which was then washed, treated with trypsin, and purified. The resulting tryptic peptides were analyzed directly by mass spectrometry. Peptide mass fingerprints were generated for the identification of the protein, and MALDI-PSD was used to sequence Fragments T3, T4, T5, and T6. The resulting data were used to search GenBank for a human protein having or a nucleic acid encoding, an amino acid sequence matching this set of peptides. HSPC194 was identified as the protein. (Table 2) presents the tryptic peptide mass fingerprints of the Tb-PCTMB target-protein, observed by MALDI-TOF MS that matches theoretical peptide masses of HSPC194, along with the corresponding amino acid sequences and residue numbers of the peptides in HSPC194.

TABLE 2
Tryptic Peptide Mass Fingerprint Analysis

| | | | | | |
|---|---|---|---|---|---|
| Fragment | Residue | | Theoretical | Observed | |
| No. | Nos. | Sequence | [M + H] | [M + H] | Δ mass |

| | | | | | |
|---|---|---|---|---|---|
| T1 | 1-31 | MQDTGSVVPLH | 3242.8 | ND* | — |
| | | WFGFGYAALVAS | | | |
| | | GGIIGYVK | | | |
| T2 | 23-60 | AGSVPSLAAGLL | 2818.2 | ND* | — |
| | | FGSLAGLGAYQL | | | |
| | | SQDPR | | | |
| T3 | 61-79 | NVWVFLATSGTL | 2025.4 | 2024.06 | 1.34 |
| | | AGIMGMR | | | |
| T4 | 80-85 | FYHSGK | 738.8 | 738.3 | 0.5 |
| T5 | 86-102 | FMPAGLIAGASLL | 1691.1 | 1689.91 | 1.19 |
| | | MVAK | | | |
| T6 | 103-112 | VGVSMIFNRPH | 1144.3 | 1142.55 | 1.75 |

*Not Determined.

Thus, Tb-PCTMB binds specifically to a single protein that is expressed in cancer cell lines. Both a monomer form (813 m/z) and dimer form (1625 m/z) of Tb-PCTMB complexed to a peptide were observed in the tryptic digests of each cell line (LNCaP, T84, Caco-2, and RBL-2H3). This data set indicates that Tb-PCTMB is not covalently attached to the protein. Confocal fluorescence microscopy verified that the Tb-PCTMB is localized throughout the cytoplasm of the cancer cell lines, but is not present in the healthy (control) cells. The gene encoding HSPC194 may be PCR-amplified from cell lines in which such binding is observed, e.g., by using oligonucleotide primers of SEQ ID NOs:3 and 4.

Table 3 below shows the peptide and oligonucleotide sequences. SEQ ID NO:1 is the DNA sequence of the chelate binding protein, HSPC194, as described herein. SEQ ID NO: 2 is the protein sequence of the chelate binding protein, HSPC194, described herein. SEQ ID NO: 3 is the DNA sequence of the forward primer for HSPC194. SEQ ID NO: 4 is the DNA sequence of the reverse primer for HSPC 194.

TABLE 3
Sequences

```
Seq. ID No. 1:
<210> 1
<211> 423
<212> DNA
<213> Homo sapiens
<220>
<221> CDS
<222> 1 . . . 420
<223> HSPC194 (chelate binding protein)
<400> 1
atg cag gac act ggc tca gta gtg cct ttg cat tgg ttt ggc ttt ggc
60
Met Gln Asp Thr Gly Ser Val Val Pro Leu His Trp Phe Gly Phe Gly
1 5 10 15
tac gca gca ctg gtt gct tct ggt ggg atc att ggc tat gta aaa gca
120
Tyr Ala Ala Leu Val Ala Ser Gly Gly Ile Ile Gly Tyr Val Lys Ala
                        20 25 30
ggc agc gtg ccg tcc ctg gct gca ggg ctg ctc ttt ggc agt cta gcc
180
Gly Ser Val Pro Ser Leu Ala Ala Gly Leu Leu Phe Gly Ser Leu Ala
                35 40 45
ggc ctg ggt gct tac cag ctg tct cag gat cca agg aac gtt tgg gtt
240
Gly Leu Gly Ala Tyr Gln Leu Ser Gln Asp Pro Arg Asn Val Trp Val
        50 55 60
ttc cta gct aca tct ggt acc ttg gct ggc att atg gga atg agg ttc
300
Phe Leu Ala Thr Ser Gly Thr Leu Ala Gly Ile Met Gly Met Arg Phe
65 70 75 80
tac cac tct gga aaa ttc atg cct gca ggt tta att gca ggt gcc agt
360
Tyr His Ser Gly Lys Phe Met Pro Ala Gly Leu Ile Ala Gly Ala Ser
                                85 90 95
ttg ctg atg gtc gcc aaa gtt gga gtt agt atg ttc aac aga ccc cat
420
Leu Leu Met Val Ala Lys Val Gly Val Ser Met Phe Asn Arg Pro His
                        100 105 110
tag
423
<210> Seq. ID No. 2
<211> 112
<212> PRT
<213> Homo sapiens
<220>
<223> HSPC194 (chelate binding protein)
<400> 2
Met Gln Asp Thr Gly Ser Val Val Pro Leu His Trp Phe Gly Phe Gly
1 5 10 15
Tyr Ala Ala Leu Val Ala Ser Gly Gly Ile Ile Gly Tyr Val Lys Ala
                        20 25 30
Gly Ser Val Pro Ser Leu Ala Ala Gly Leu Leu Phe Gly Ser Leu Ala
                35 40 45
Gly Leu Gly Ala Tyr Gln Leu Ser Gln Asp Pro Arg Asn Val Trp Val
        50 55 60
Phe Leu Ala Thr Ser Gly Thr Leu Ala Gly Ile Met Gly Met Arg Phe
65 70 75 80
Tyr His Ser Gly Lys Phe Met Pro Ala Gly Leu Ile Ala Gly Ala Ser
                                85 90 95
Leu Leu Met Val Ala Lys Val Gly Val Ser Met Phe Asn Arg Pro His
                        100 105 110
<210> Seq. ID No. 3
<211> 18
<212> DNA
<213> Artificial Sequence
<220>
<223> Forward Primer for HSPC194 PCR
<400> 3
tggtaccttg gctggcat
18
<210> Seq. ID No. 4
<211> 21
<212> DNA
<213> Artificial Sequence
<223> Reverse Primer for HSPC194 PCR
<400> 4
ctaatggggt ctgttgaaca t
21
```

### Example 32

### In Vitro Permeability of Eu-QCTME to Cancer Cells

Eu-QCTME (Eu(III) N-(6-methyl-2-quinolylmethyl)-N′,N″,N′″-tris(methylene phosphonic acid ethyl ester)-1,4,7,10-tetraazacyclododecane) has been found to be permeable to abnormal and disease-state cells. Permeability of Eu-QCTME to epithelial cancer cell lines, LNCaP, T84, Caco-2, and RBL was measured through detection of the intensity of its inherent fluorescence (excitation wavelength of 330 nm, emission at 610 nm).

Cells were grown as described herein (Example 1). Cells were grown to confluence in T-150 flask (about 2×10⁶ cells). Before treating with Eu-QCTME, cells were rinsed twice with warm 10 mM HEPES (Gibco BRL, Grand Island N.Y.), and then treated with either 5 mL of 0, 1, 500, 1000, or 2000 μM Eu-QCTME in 10 mM HEPES, pH 7.4, 5 mM KCl, 150 mM NaCl, 0.7 mM NaH₂PO₄ for 2 hours at 37° C. (dose response study) or with 5 mL of Eu-QCTME (1 mM final concentration) for the following duration: 0, 1, 2, 4, and 8 hours (time course study). After treatment, the Eu-QCTME supernatant was discarded and the cells were rinsed three times at room temperature with 10 mM HEPES. Cells were then removed from the flask by manual scraping. Cells were transferred to a 50-mL conical tube and centrifuged (400×g) for 5 minutes and the test material removed. The resulting cell pellet was isolated for cytoplasmic and membrane fractions and was analyzed by measuring the inherent fluorescence of the Eu-QCTME compound.

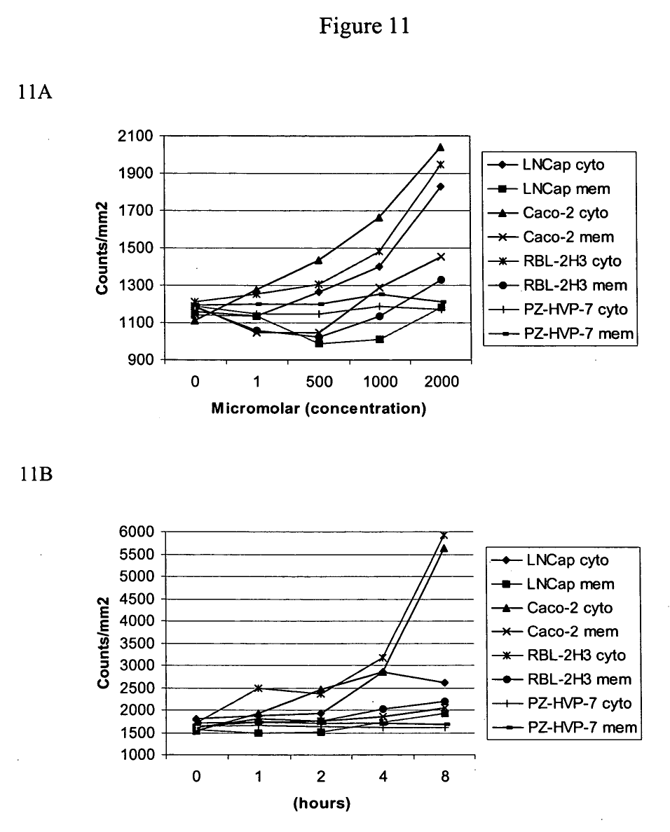

Dose response curves and time course curves for each cell line were generated, (FIGS. 11A and 11B). Carcinoma cell lines, LNCaP, Caco-2, and RBL-2H3, exhibited significant increases in fluorescence in the cytoplasm as the concentration of increased Eu-QCTME compared to a normal cell line, PZ-HPV-7. In the membrane fractions, Caco-2 and RBL-2H3 have detectable quantity of fluorescence and show an increase in fluorescence as the concentration increases compared to the normal cell line, PZ-HPV-7.

Eu-QCTME therefore is permeable to cancer cell membranes with the majority of the fluorescence associated with the cytoplasm versus the membrane fractions. More importantly, Eu-QCTME is significantly less detectable in normal cells, further supporting the specificity to cancer cells over normal cell lines.

Furthermore, one analogue, QCTMP (FIG. 26), having lesser lipophilic character (a free phosphonic acid moiety) was evaluated. To explore the effect of reducing the lipophilicity of this family of chelate structures, LNCaP, Caco-2, T84, RBL-2H3, and PZ-HPV-7 were tested to determine their dose response to Eu-QCTME and Eu-QCTMP. In this study, the cytoplasm and membrane fractions were extracted by Mem-PER Eukaryotic Membrane Protein Extraction Reagent Kit as described by the manufacturers (Pierce Biotechnology, Rockford, Ill.) and analyzed by a Fluor-S Multilmager. Dose responses for each cell line were generated, where the cells were incubated for 2 h with 1 mM with either Eu-QCTME or Eu-QCTMP (Table 4).

TABLE 4
Dose response for each cell line incubated
with Eu-QCTME and Eu-QCTMP.
Table 4. Cancer Cell Dose Responses to Chelates

| | |
|---|---|
| Cancer | Fluorescence/mg |

| Cell Line | Chelate | Cytoplasm | Membrane |
|---|---|---|---|
| LNCaP | Eu-QCTME | 3881 | 2561 |
| | Eu-QCTMP | 2287 | 2301 |
| T84 | Eu-QCTME | 4548 | 2307 |
| | Eu-QCTMP | 2230 | 2259 |
| Caco-2 | Eu-QCTME | 4339 | 2524 |
| | Eu-QCTMP | 2300 | 2314 |
| RBL-2H3 | Eu-QCTME | 4415 | 2644 |
| | Eu-QCTMP | 2311 | 2311 |
| PZ-HVP-7 | Eu-QCTME | 2247 | 2293 |
| | Eu-QCTMP | 2276 | 2271 |

Table 4. Cancer Cell Dose Responses to Chelates

Fluorescence was significantly higher in the cytoplasm of LNCaP, Caco-2, and T84 cell lines in the presence of Eu-QCTME. More importantly Eu-QCTMP did not appear to be permeable to membranes and thus are not detectable in the cytoplasm. Additionally, Eu-QCTMP did not show any specificity or selectivity for cancer cells. Collectively, these data indicate that the lipophilic nature of Eu-QCTME is important in the chelate's ability to cross the membrane into the cytoplasm, further illustrating the importance of the structural architecture of the chelating agent. While not wishing to be limited to any particular theory, it is possible, based upon these results, that Eu-QCTMP lacks sufficient lipophilic character (all the alkyl groups are eliminated) to interact with tissue morphology. Optionally, Eu-QCTMP has an increased anionic charge as a result of the elimination of the alkyl groups, possibly further diminishing its permeability into tissues and/or cells.

### Example 33

### Permeability of Tb-PCTMB and Eu-QCTME to Apoptotic Cells

Both Tb-PCTMB and Eu-QCTME were found to be permeable to etoposide-induced HEK293 cells. Permeability of Tb-PCTMB and Eu-QCTME was measured through detection of the intensity of their respective fluorescence.

The human kidney transformed HEK293 cells (obtained from the ATCC) were grown in Eagle's Minimum Essential Medium (EMEM) supplemented with 10% (v/v) heat-inactivated fetal bovine serum, sodium pyruvate, nonessential amino acids and antibiotics in a humidified 5% CO₂ atmosphere at 37° C. Medium and supplements were purchased from Gibco bRL (Grand Island, N.Y.). Etoposide was obtained from Sigma (St. Louis, Mo.) and prepared as 20 mM solution in dimethyl sulfoxide (DMSO). For treatments, etoposide was applied to the HEK293 cultures at 1:1000 dilution (final concentration of 20 μM) after cell became 75% confluent. As a control, cells were fed with medium containing 0.1% DMSO in the absence of etoposide. After treatment with etoposide, cells were incubated in a humidified 5% CO₂ at 37° C. for 24 hours. HEK293 cells were collected by removing treatment medium and washed 2 times with 10 mM HEPES buffer. Final cell pellets were resuspended in a small amount of HEPES and stored at 4° C. for further study.

Cells were grown to confluence in T-150 flasks (about 2×10⁶ cells). Before treating with a specific chelate, cells were rinsed twice with warm 10 mM HEPES (Gibco BRL, Grand Island N.Y.), and then treated with 1 mM chelate (e.g., Tb-PCTMB, Tb-PCTMM, Tb-PCTA, Eu-QCTME or Eu-QCTMP, as shown in FIG. 26) in 10 mM HEPES, pH 7.4, 5 mM KCl, 150 mM NaCl, 0.7 mM NaH₂PO₄ for 2 hours at 37° C. After treatment, the chelate or analogue supernatant was discarded and the cells were rinsed three times at room temperature with 10 mM HEPES. Cells were then removed from the flask by manual scraping. Cells were transferred to a 50-mL conical tube and centrifuged (400×g) for 5 minutes and the test material removed. Remaining cells were rinsed twice with cold 10 mM HEPES. Cells were used in cell binding assays. Flasks that had the majority of the cells detach during treatment were removed, centrifuged and washed as described previously. The resulting cell pellet was isolated for cytoplasmic and membrane fractions and was analyzed by measuring the inherent fluorescence of the Tb-PCTMB compound.

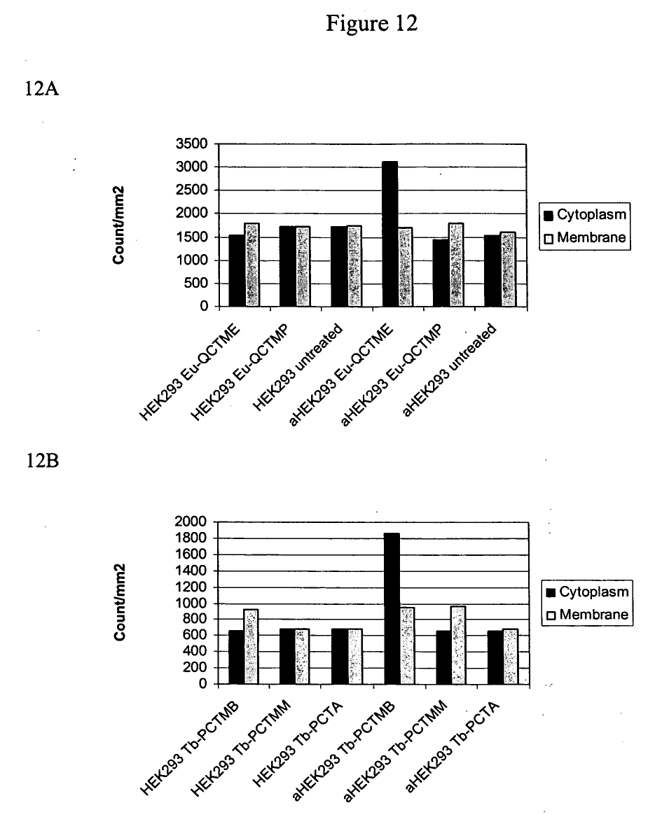

Fluorescence of both Eu-QCTME and Tb-PCTMB was detected in the cytoplasm fraction of etoposide-induced HEK293 cells treated with the apoptotic trigger (FIGS. 12A and 12B). By contrast, cultures of non-induced HEK293 cells treated with either Eu-QCTME or Tb-PCTMB exhibited a very low fluorescence in the cytoplasm fraction.

Tb-PCTMB and Eu-QCTME therefore are permeable to apoptotic induced HEK293 cells. In contrast, the less lipophilic analogues, Eu-QCTMP, Tb-PCTMM, and Tb-PCTA (FIG. 26), did not show any significant differential fluorescence when comparing cytoplasm and membrane fractions in either etoposide-induced or normal HEK293 cells (Table 5). Thus, the lipophilic nature of Eu-QCTME and Tb-PCTMB is (both having a logp value between 0 and 4; and with both having three phosphonate esters) is a distinguishing feature that permits these chelates to cross or be permeable to the abnormal cell membrane.

TABLE 5
Dose response for HEK293 cells

Fluorescence/mg

| Cells Tested | Chelate | Cytoplasm | Membrane |
|---|---|---|---|
| HEK293 | Eu-QCTME | 1527 | 1799 |
| Normal | Eu-QCTMP | 1732 | 1738 |
| | untreated | 1734 | 1756 |
| HEK293 | Eu-QCTME | 3119 | 1697 |
| Apoptotic | Eu-QCTMP | 1450 | 1800 |
| | untreated | 1527 | 1610 |
| HEK293 | Tb-PCTMB | 652 | 925 |
| Normal | Tb-PCTMM | 682 | 679 |
| | Tb-PCTA | 686 | 685 |
| HEK293 | Tb-PCTMB | 1863 | 949 |
| Apoptotic | Tb-PCTMM | 656 | 962 |
| | Tb-PCTA | 652 | 678 |

### Example 34

### In Vivo Permeability of Tb-PCTMB and Eu-QCTME to Tumors

Tb-PCTMB and Eu-QCTME were found permeable to in vivo epithelia cancer tissue, as measured by their respective inherent fluorescence. Epithelial cancer was induced in the right buccal cheek pouch of Golden Syrian Hamster by swabbing 0.5% DMBA (7/12-dimethylbenz[a]anthracene) in mineral oil solution. This procedure was repeated approximately three times a week for up to 20 weeks. Small lesions were visible within seven weeks. Hamsters were anesthetized using 25-mg ketamine and 0.25 mg xylazine delivered IP with redosing with half that dose after 1.5 hours.

A 2 mM solution of either Tb-PCTMB or Eu-QCTME solution was prepared in 5% ethanol and 95% water. Mild heating was required to achieve dissolution. One milliliter of chelate-solution was applied topically to the cheek pouch over a 10 minute period. After 10 minutes the pouch was washed with 5% ethanol for 30 seconds. Images were acquired after treatment and washes. The resulting treated DMBA-induced tumor tissue and treated normal tissue was excised from the hamster for analysis of cytoplasmic and membrane fractions.

Duplicate excised tissue samples were separated into cytoplasm and membrane fractions by extraction using the MEM-PER Eukaryotic Membrane Protein Extraction Reagent Kit as described by manufacturers (Pierce Biotechnology, Rockford, Ill.). The resulting fractions were analyzed for chelate fluorescence using a FLUOR-S MULTIIMAGER (Bio-Rad Laboratories, Inc., Hercules, Calif., USA). Readings were Adjusted Volume CNT*mm2. Results are reported in Table 6. Both Tb-PCTMB and Eu-QCTME, exhibited significantly greater fluorescence in the cytoplasm of tumor tissue compared to the cytoplasmic fraction from normal tissue.

TABLE 6
Localization of Chelates in Cytoplasm vs Membranes

| | |
|---|---|
| Chelate | Fluorescence/mg |

| Treatment | Tissue Sample | Cytoplasm | Membrane |
|---|---|---|---|
| Eu-QCTME | Tumor 1 | 3524 | 1670 |
| | Tumor 2 | 3659 | 1676 |
| | Normal 1 | 1344 | 1348 |
| | Normal 2 | 1369 | 1374 |
| Tb-PCTMB | Tumor 1 | 3014 | 1331 |
| | Tumor 2 | 3064 | 1484 |
| | Normal 1 | 1138 | 1126 |
| | Normal 2 | 1117 | 1125 |

### Example 35

### Adsorption and Permeability of Chelant Compounds in In Vitro Cell Lines

As a prerequisite for the understanding of the functional attributes of Tb-PCTMB and Eu-QCTME for diagnostics and therapeutic purposes, and for other uses, it is useful to synthesize a series of chelate compounds that contain structural elements of both PCTMB and QCTME and test these compounds for adsorption and permeability to different cell lines. The first approach was to test for adsorption to a normal cell line (HEK293) and to two epithelial cancer cell lines (LNCaP and Caco-2). The second approach, described in this example, involves testing only the compounds that adsorbed in the cancer cell lines and not in the normal cell lines for permeability. Adsorption and permeability of the compounds to epithelial cancer cell lines and a normal cell line was measured through the detection of the compound by HPLC and mass spectrometry.

Cell cultures. LNCaP cell line is a human prostate carcinoma. Cells were grown in RPMI 1640 Medium supplemented 10% FBS, 2 mM L-glutamine, glucose (2 g/L) augmented with gentamicin (50 μg/mL), penicillin/streptomycin (100 IU/mL/100 μg/mL) and amphotericin B (2.5 μg/mL). Caco-2 cell line is a human colorectal carcinoma. These cells were grown in Eagle's Minimum Essential Medium supplemented with 20% FBS, Earle's BSS, 2 mM L-glutamine, 1.0 mM sodium pyruvate, 0.1 mM nonessential amino acids augmented with gentamicin (50 μg/mL), penicillin/streptomycin (100 IU/mL/100 μg/mL), and amphotericin B (2.5 μg/mL). The human kidney transformed cells HEK293 were grown in Eagle's Minimum Essential Medium (EMEM) supplemented with 10% (v/v) heat-inactivated fetal bovine serum, sodium pyruvate, nonessential amino acids and antibiotics in a humidified 5% CO₂ atmosphere at 37° C. Medium and supplements were purchased from Gibco BRL (Grand Island, N.Y.) and all cell lines were obtained from ATCC.

Detection of chelate compounds for the determination of adsorption. Cells were grown to confluence in T-150 flasks (about 2×10⁷ cells). Before treating with chelate compounds, cells were rinsed twice with warm 10 mM HEPES (Gibco BRL, Grand Island N.Y.), removed from the flask, and transferred to a 50-mL conical tube and centrifuged (400×g) for 5 minutes. The resulting cells were treated with individual chelate compounds with 50 gM of chelate compounds in 10 mM HEPES, pH 7.4, 5 mM KCl, 150 mM NaCl, 0.7 mM NaH₂PO₄ for 0, 30, and 90 minutes at 37 ° C. After treatment, cells were pelleted by centrifugation and the resulting solution was analyzed by a combination of HPLC and mass spectrometry.

The following compounds were tested:

| | |
|---|---|
| 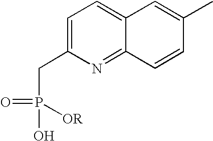 | 1 R = Me 2 R = Et 3 R = n-Bu |
| 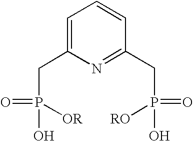 | 4 R = Me 5 R = Et 6 R = n-Bu |
| 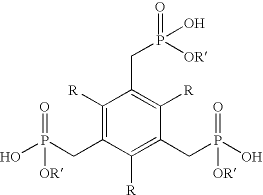 | 7 R = H, R′ = Me 8 R = H, R′ = Et 9 R = H, R′ = n-Bu 10 R = Me, R′ = Me 11 R = Me, R′ = Et 12 R = Me, R′ = n-Bu |
| 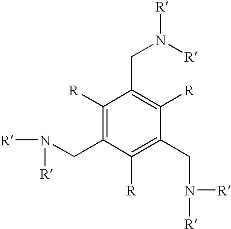 | 13 R = H, R′ = Me 14 R = H, R′ = Et, 15 R = H, R′ = n-Bu 16 R = Me, R′ = Me 17 R = Me, R′ = Et 18 R = Me, R′ = n-Bu |
| 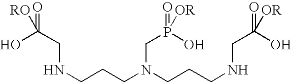 | 19 R = Me 20 R = Et, 21 R = n-Bu |
| 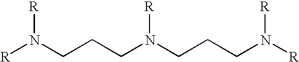 | 22 R = Me 23 R = Et, 24 R = n-Bu |

Time course curves for each cell line were generated. Five compounds (2, 3, 8, 9, and 12) were shown to be adsorbed in carcinoma cell lines Caco-2 and LNCaP (Table 7). More importantly, these compounds (2, 3, 8, 9, and 12) were not adsorbed in normal cells (HEK293), indicating specificity to cancer cells over normal cell lines. Furthermore, analogues (1, 7, and 10 in Table 7), with decreasing lipophilic character were evaluated and did not adsorb to either cancer cells or normal cells. In addition, seven compounds (4, 6, 19, 20, 21, 22, and 23) adsorbed to both the cancer cell lines and the normal cell line. Eight compounds did not adsorb to any of the cell lines tested, however it is possible that these compounds could be active in other assays.

TABLE 7
Adsorption of chelate compounds in in vitro cell lines.

| | | | Adsorption | | |
|---|---|---|---|---|---|
| Sample ID | | Solubility | HEK293 | Caco-2 | LNCaP |
| 1 | R = Me | S^{a} | 0 | 0 | 0 |
| 2 | R = Et | S | 0 | ++ | ++ |
| 3 | R = n-Bu | S | 0 | ++ | ++++ |
| 4 | R = Me | S | ++ | ++ | ++ |
| 5 | R = Et | S | + | 0 | + |
| 6 | R = n-Bu | S | +++ | +++ | +++ |
| 7 | R = H, R′ = Me | S | 0 | 0 | 0 |
| 8 | R = H, R′ = Et | S | 0 | +++ | +++ |
| 9 | R = H, R′ = n-Bu | S | 0 | ++ | ++ |
| 10 | R = Me, R′ = Me | S | 0 | 0 | 0 |
| 11 | R = Me, R′ = Et | S | 0 | 0 | 0 |
| 12 | R = Me, R′ = | S | 0 | ++++ | +++ |
| | n-Bu | | | | |
| 13 | R = H, R′ = Me | S | 0 | 0 | 0 |
| 14 | R = H, R′ = Et | S | 0 | 0 | 0 |
| 15 | R = H, R′ = n-Bu | I^{b} | nt | nt | nt |
| 16 | R = Me, R′ = Me | S | 0 | 0 | 0 |
| 17 | R = Me, R′ = Et | I | nt | nt | nt |
| 18 | R = Me, R′ = | I | nt | nt | nt |
| | n-Bu | | | | |
| 19 | R = Me | S | ++ | ++ | ++ |
| 20 | R = Et | S | ++ | ++ | ++ |
| 21 | R = n-Bu | S | ++ | ++ | ++ |
| 22 | R = Me | S | + | ++ | + |
| 23 | R = Et | S | + | ++ | + |
| 24 | R = n-Bu | I | nt | nt | Nt |

^{a}S = soluble in <5% methanol, ethanol, or DMSO.
^{b}I = insoluble >10 mM.
nd = not tested
Grading Scale represents % of chelate compound adsorbed relative to time zero where (0 = 0-10%; + = 11-20%; ++ = 21-40%; +++ = 41-60%; ++++ = >60%)

Detection of chelant compounds for the determination of permeability. Cells were grown to confluence in T-150 flasks (about 2×10⁷ cells). Before treating with chelate compounds, cells were rinsed twice with warm 10 mM HEPES (Gibco BRL, Grand Island N.Y.), removed from the flask, and transferred to a 50-mL conical tube and centrifuged (400×g) for 5 minutes. The resulting cells were treated with individual chelate compounds with 50 μM of chelate compounds in 10 mM HEPES, pH 7.4, 5 mM KCl, 150 mM NaCl, 0.7 mM NaH₂PO₄ for 90 minutes at 37° C. After treatment, cells were pelleted by centrifugation, the cytoplasm was extracted by Mem-PER Eukaryotic Membrane Protein Extraction Reagent Kit as described by manufacturers (Pierce biotechnology, Rockford, Ill.) and the resulting solution was analyzed by a combination of HPLC and mass spectrometry (Table 8).

TABLE 8
Permeability of chelate compounds in in vitro cell lines.

| | Permeability^{a} | | |
|---|---|---|---|
| Sample ID | HEK293 | Caco-2 | LNCaP |
| 2 | 0^{b} | + | + |
| 3 | 0 | + | + |
| 8 | 0 | + | + |
| 9 | 0 | + | + |
| 12 | 0 | + | + |

^{a}Permeability = detection of chelate compounds in the cytoplasm.
^{b}Absence = 0 and presence = +.

The five compounds (2, 3, 8, 9, and 12) shown previously to adsorb to carcinoma cell lines also were detected within the cytoplasm suggesting that these compounds were permeable. More importantly, compared to results seen in normal cells (non-cancerous control cells), these tests detected little to no permeability of the 5 compounds to cell membranes. Collectively, these data verify that the lipophilic nature of these 5 compounds are important in the compounds ability to cross the membrane into the cytoplasm. Thus, the key physico-chemical features of these compounds for disease-specific intracellular uptake (lipophilicity) are well illustrated by these data.

### Example 36

### Identification of Novel Target-chelate Compound Associations

Interaction of Tb-PCTMB and HSPC194 Protein—Tb-PCTMB, Eu-QCTME and 24 chelate compounds were screened to elucidate structure/fuction relationships with purified HSPC194 protein. Purified HSPC194 protein was captured on an affinity support column. The unbound contaminants, which have no affinity for the ligand, are washed through the column, leaving the HSPC194 protein bound to the matrix. HSPC194 protein is eluted by adding the different chelate compounds or Tb-PCTMB and Eu-QCTME that competes for the bound ligand or changes the steric structure of the protein. Eluted samples were analyzed by high resolution SDS-PAGE, thus identifying one major protein band with an apparent molecular weight of 15 kDa as identified by Coomassie staining. Surprisingly, Tb-PCTMB at 25, 50, and 500 μM concentration was the only compound to elute HSPC194 from the bound matrix (Table 9).

TABLE 9
Elution of HSPC194 by different compounds

| | | Elution of | |
|---|---|---|---|
| Compound | | HSPC194 | |
| ID | 500 μM | 50 μM | 10 μM |
| Eu-QCTME | 0 | 0 | 0 |
| Tb-PCTMB | ++++ | +++ | ++ |
| 1 | 0 | nd | nd |
| 2 | 0 | nd | nd |
| 3 | 0 | nd | nd |
| 4 | 0 | nd | nd |
| 5 | 0 | nd | nd |
| 6 | 0 | nd | nd |
| 7 | 0 | nd | nd |
| 8 | 0 | nd | nd |
| 9 | 0 | nd | nd |
| 10 | 0 | nd | nd |
| 11 | 0 | nd | nd |
| 12 | 0 | nd | nd |
| 13 | 0 | nd | nd |
| 14 | 0 | nd | nd |
| 15 | 0 | nd | nd |
| 16 | 0 | nd | nd |
| 17 | 0 | nd | nd |
| 18 | 0 | nd | nd |
| 19 | 0 | nd | nd |
| 20 | 0 | nd | nd |
| 21 | 0 | nd | nd |
| 22 | 0 | nd | nd |
| 23 | 0 | nd | nd |
| 24 | 0 | nd | nd |

Identification of novel target-chelate compound associations—Caco-2 and LNCaP cell lines were lysed by sonication or by lysis in hypotonic buffer. Soluble proteins were enriched and captured on an affinity support column. The unbound proteins, which have no affinity for the ligand, are washed through the column. Proteins were eluted by adding different chelate compounds that compete for the bound ligand or change the steric structure of a given protein(s). Eluted samples were analyzed by high resolution SDS-PAGE. The protein-chelate compound associations were further analyzed to identify the different proteins by excising the corresponding protein bands from the gel, which was then treated with trypsin and purified. Peptide mass fingerprints were generated for the identification of the proteins, and MALDI-PSD was used to generate sequence-tags. The resulting data was used to search GeneBank for human proteins having or a nucleic acid encoding, an amino acid sequence matching specific sets of peptides. Table 10 presents proteins identified by elution of target-chelate compound associations.

TABLE 10
Elution and identification of novel
target-chelate compound associations.

| Compound ID | Protein ID | MW | Caco-2 | LNCaP |
|---|---|---|---|---|
| 2 | BiP | 75 | + | + |
| | Amphiphysin I | 70 | + | + |
| 3 | BiP | 75 | + | + |
| | Amphiphysin I | 70 | + | + |
| 8 | Cytokeratin 8 | 42 | + | + |
| | Keratin 18 | 38 | + | + |
| 9 | Cytokeratin 8 | 42 | + | + |
| | Keratin 18 | 38 | + | + |
| 12 | Cytokeratin 8 | 42 | + | + |
| | Keratin 18 | 38 | + | + |

Thus, chelate-compounds (2 and 3) bind specifically to two different proteins, BiP (Seq. ID No. 5), and amphiphysin I (Seq. ID No. 7) that are expressed in at least two cancer cell lines. Chelate-compounds (8, 9 and 12) bind specifically to at least two different proteins that are also expressed in at least two cancer cell lines, cytokeratin 8 (Seq. ID No. 10) and keratin 18 (Seq. ID No. 9). These two sets of chelate-compounds associate to different proteins, suggesting different mechanisms.

Table 11 below shows the amino acid sequence of the proteins.

TABLE 11

Identification of proteins associated with the following
compounds: (A) protein A (compounds 2 and 3), (B) protein
B (compounds 2 and 3), (C) protein C (compounds 8, 9, and
12), and (D) protein D (compounds 8, 9, and 12).

A.

| | | | Seq |
|---|---|---|---|
| Protein | | Accession | ID |
| [Species] | Amino Acid sequence | No. | No. |
| 78 KD Glucose- | 1 mklslvaaml lllsaaraee edkkedvgtv | P11021 | Seq |
| regulated protein | vgidlgttys cvgvfkngrv eiiandqgnr | | ID |
| precursor (BiP) | 61 itpsyvaftp egerligdaa knqltsnpen | | No. |
| [Homo sapiens] | tvfdakrlig rtwndpsvqq dikflpfkvv | | 5 |
| | 121 ekktkpyiqv digggqtktf apeeisamvl | | |
| | tkmketaeay lgkkvthavv tvpayfndaq | | |
| | 181 rqatkdagti aglnvmriin eptaaaiayg | | |
| | ldkregekni lvfdlgggtf dvslltidng | | |
| | 241 vfevvatngd thlggedfdq rvmehfikly | | |
| | kkktgkdvrk dnravqklrr evekakalss | | |
| | 301 qhqarieies fyegedfset ltrakfeeln | | |
| | mdlfrstmkp vqkvledsdl kksdideivl | | |
| | 361 vggstripki qqlvkeffng kepsrginpd | | |
| | eavaygaavq agvlsgdqdt gdlvllhvcp | | |
| | 421 ltlgietvgg vmtklipsnt vvptknsqif | | |
| | stasdnqptv tikvyegerp ltkdnhllgt | | |
| | 481 fdltgippap rgvpqievtf eidvngilrv | | |
| | taedkgtgnk nkititndqn rltpeeierm | | |
| | 541 vndaekfaee dkklkeridt rnelesyays | | |
| | lknqigdkek lggklssedk etmekaveek | | |
| | 601 iewleshqda diedfkakkk eleeivqpii | | |
| | sklygsagpp ptgeedtaek del | | |
| | | | Seq |
| Gene | | Accession | ID |
| [Species] | cDNA Sequence | No. | No. |
| Human 78 | 1 atgaagctct ccctggtggc cgcgatgctg | M19645 | Seq |
| kdalton | ctgctgctca gcgcggcgcg ggccgaggag | | ID |
| glucose- | 61 gaggacaaga aggaggacgt gggcacggtg | | No. |
| regulated | gtcggcatcg acttggggac cacctactcc | | 6 |
| protein | 121 tgcgtcggcg tgttcaagaa cggccgcgtg | | |
| (GRP78) | gagatcatcg ccaacgatca gggcaaccgc | | |
| gene [Homo | 181 atcacgccgt cctatgtcgc cttcactcct | | |
| sapiens] | gaaggggaac gtctgattgg cgatgccgcc | | |
| | 241 aagaaccagc tcacctccaa ccccgagaac | | |
| | acggtctttg acgccaagcg gctcatcggc | | |
| | 301 cgcacgtgga atgacccgtc tgtgcagcag | | |
| | gacatcaagt tcttgccgtt caaggtggtt | | |
| | 361 gaaaagaaaa ctaaaccata cattcaagtt | | |
| | gatattggag gtgggcaaac aaagacattt | | |
| | 421 gctcctgaag aaatttctgc catggttctc | | |
| | actaaaatga aagaaaccgc tgaggcttat | | |
| | 481 ttgggaaaga aggttaccca tgcagttgtt | | |
| | actgtaccag cctattttaa tgatgcccaa | | |
| | 541 cgccaagcaa ccaaagacgc tggaactatt | | |
| | gctggcctaa atgttatgag gatcatcaac | | |
| | 601 gagcctacgg cagctgctat tgcttatggc | | |
| | ctggataaga gggaggggga gaagaacatc | | |
| | 661 ctggtgtttg acctgggtgg cggaaccttc | | |
| | gatgtgtctc ttctcaccat tgacaatggt | | |
| | 721 gtcttcgaag ttgtggccac | | |
| | taatggagat actcatctgg gtggagaaga | | |
| | ctttgaccag 781 cgtgtcatgg | | |
| | aacacttcat caaactgtac aaaaagaaga | | |
| | cgggcaaaga tgtcaggaag 841 | | |
| | gacaatagag ctgtgcagaa actccggcgc | | |
| | gaggtagaaa aggccaaggc cctgtcttct | | |
| | 901 cagcatcaag caagaattga aattgagtcc | | |
| | ttctatgaag gagaagactt ttctgagacc | | |
| | 961 ctgactcggg ccaaatttga agagctcaac | | |
| | atggatctgt tccggtctac tatgaagccc | | |
| | 1021 gtccagaaag tgttggaaga ttctgatttg | | |
| | aagaagtctg atattgatga aattgttctt | | |
| | 1081 gttggtggct cgactcgaat tccaaagatt | | |
| | cagcaactgg ttaaagagtt cttcaatggc | | |
| | 1141 aaggaaccat cccgtggcat aaacccagat | | |
| | gaagctgtag cgtatggtgc tgctgtccag | | |
| | 1201 gctggtgtgc tctctggtga tcaagataca | | |
| | ggtgacctgg tactgcttca tgtatgtccc | | |
| | 1261 cttacacttg gtattgaaac tgtaggaggt | | |
| | gtcatgacca aactgattcc aagtaataca | | |
| | 1321 gtggtgccta ccaagaactc | | |
| | tcagatcttt tctacagctt ctgataatca | | |
| | accaactgtt 1381 acaatcaagg | | |
| | tctatgaagg tgaaagaccc ctgacaaaag | | |
| | acaatcatct tctgggtaca 1441 | | |
| | tttgatctga ctggaattcc tcctgctcct | | |
| | cgtggggtcc cacagattga agtcaccttt | | |
| | 1501 gagatagatg tgaatggtat tcttcgagtg | | |
| | acagctgaag acaagggtac agggaacaaa | | |
| | 1561 aataagatca caatcaccaa tgaccagaat | | |
| | cgcctgacac ctgaagaaat cgaaaggatg | | |
| | 1621 gttaatgatg ctgagaagtt | | |
| | tgctgaggaa gacaaaaagc tgaaggagcg | | |
| | cattgatact 1681 agaaatgagt | | |
| | tggaaagcta tgcctattct ctaaagaatc | | |
| | agattggaga taaagaaaag 1741 | | |
| | ctgggaggta aactttcctc tgaagataag | | |
| | gagaccatgg aaaaagctgt agaagaaaag | | |
| | 1801 attgaatggc tggaaagcca ccaagatgct | | |
| | gacattgaag acttcaaagc taagaagaag | | |
| | 1861 gaactggaag aaattgttca accaattatc | | |
| | agcaaactct atggaagtgc aggccctccc | | |
| | 1921 ccaactggtg aagaggatac agcagaaaaa | | |
| | gatgagttgt ag | | |

B.

| | | | Seq |
|---|---|---|---|
| Protein | | Accession | ID |
| [Species] | Amino Acid Sequence | No. | No. |
| amphiphysin I | 1 madiktgifa knvqkrlnra | AAC02977 | Seq |
| [Homo sapiens] | qekvlqklgk adetkdeqfe | | ID |
| | eyvqnfkrqe aegtrlqrel | | No. |
| | 61 rgylaaikgm qeasmkltes | | 7 |
| | lhevyepdwy gredvkmvge | | |
| | kcdvlwedfh qklvdgsllt | | |
| | 121 ldtylgqfpd iknriakrsr | | |
| | klvdydsarh hlealqsskr | | |
| | kdesriskae eefqkaqkvf | | |
| | 181 eefnvdlqee lpslwsrrvg | | |
| | fyvntfknvs sleakfhkei | | |
| | avlchklyev mtklgdqhad | | |
| | 241 kaftiqgaps dsgplriakt | | |
| | psppeepspl psptaspnht | | |
| | lapaspapar prspsqtrkg | | |
| | 301 ppvpplpkvt ptkelqqeni | | |
| | isffednfvp eisvttpsqn | | |
| | evpevkkeet lldldfdpfk | | |
| | 361 pevtpagsag vthspmsqtl | | |
| | pwdlwttstd lvqpasggsf | | |
| | ngftqpqdts lftmqtdqsm | | |
| | 421 icnliipgad adaavgtlvs | | |
| | aaegapgeea eaekatvpag | | |
| | egvsleeaki gtettegaes | | |
| | 481 aqpeaeelea tvpqekvips | | |
| | vviepasnhe eegeneitig | | |
| | aepketteda appgptsetp | | |
| | 541 elateqkpiq dpqptpsapa | | |
| | mgaadqlasa reasqelppg | | |
| | flykvetlhd feaansdelt | | |
| | 601 lqrgdvvlvv psdseadqda | | |
| | gwlvgvkesd wlqyrdlaty | | |
| | kglfpenftr rid | | |
| | | | Seq |
| Gene | | Accession | ID |
| [Species] | cDNA Seqence | No. | No. |
| amphiphysin I | 1 atggccgaca tcaagacggg catcttcgcc | AAC02977 | Seq |
| [Homo | aagaacgtcc agaagcgact caaccgcgcg | | ID |
| sapiens] | 61 caggaaaagg tcctccaaaa gctggggaaa | | No. |
| | gctgatgaga caaaagacga acagttcgaa | | 8 |
| | 121 gaatatgtcc agaacttcaa acggcaagaa | | |
| | gcagagggta ccagacttzca gcgagaactc | | |
| | 181 cgaggatatt tagcagcaat caaaggcatg | | |
| | caggaggcct ccatgaagct cacagagtcg | | |
| | 241 ctgcatgaag tctatgagcc tgactggtat | | |
| | gggcgggaag atgtgaaaat ggttggtgag | | |
| | 301 aaatgtgatg tgctgtggga agacttccat | | |
| | caaaaactcg tggatgggtc cttgctaaca | | |
| | 361 ctggatacct acctggggca atttcctgac | | |
| | ataaagaatc gcatcgccaa gcgcagcagg | | |
| | 421 aagctagtgg actatgacag tgcccgccac | | |
| | catctggaag ctctgcagag ctccaagagg | | |
| | 481 aaggatgaga gtcgaatctc taaggcagaa | | |
| | gaagaatttc agaaagcaca gaaagtgttt | | |
| | 541 gaagagttta acgttgactt acaagaagag | | |
| | ttaccatcat tatggtcaag acgagttgga | | |
| | 601 ttttatgtta atactttcaa aaacgtctcc | | |
| | agccttgaag ccaagtttca taaggaaatt | | |
| | 661 gcggtgcttt gccacaaact gtatgaagtg | | |
| | atgacaaaac tgggtgacca gcacgccgac | | |
| | 721 aaggccttca ccatccaagg agcgcccagt | | |
| | gattcgggtc ctctccgcat tgcaaagaca | | |
| | 781 ccatcaccgc ctgaggagcc ttcacccctc | | |
| | ccgagcccga cagcaagtcc aaatcataca | | |
| | 841 ttagcacctg cgtctcccgc accagcacgg | | |
| | cctcggtcac cttcacagac aaggaaaggg | | |
| | 901 cctcctgtcc cacctctacc taaagtcacc | | |
| | ccgacaaagg aactgcagca ggagaacatc | | |
| | 961 atcagtttct ttgaggacaa ctttgttcca | | |
| | gaaatcagtg tgacaacacc ttcccagaat | | |
| | 1021 gaagtccctg aggtgaagaa agaggagact | | |
| | ttgctggatc tggactttga tcctttcaag | | |
| | 1081 cccgaggtga cacctgcagg ttctgctgga | | |
| | gtgacccact cacccatgtc tcagacattg | | |
| | 1141 ccctgggacc tatggacgac aagcactgat | | |
| | ttggtacagc cggcttctgg tggttcattt | | |
| | 1201 aatggattca cacagcccca ggatacttca | | |
| | ttattcacaa tgcagacaga ccagagtatg | | |
| | 1261 atctgcaact tgatcatacc tggagctgat | | |
| | gctgatgcag ctgttggaac cttggtgtca | | |
| | 1321 gcagctgagg gggccccagg agaggaagca | | |
| | gaggcggaga aggccactgt ccctgccggg | | |
| | 1381 gaaggagtaa gtttagagga ggccaaaatt | | |
| | ggaactgaaa ccactgaggg tgcagagagt | | |
| | 1441 gcccaacctg aagcagagga gctcgaagca | | |
| | acagtgcctc aggagaaggt cattccttcg | | |
| | 1501 gtggtcatag agcctgcctc caaccatgaa | | |
| | gaggaaggag aaaacgaaat aactataggt | | |
| | 1561 gcagagccca aggagaccac cgaggacgcg | | |
| | gctcctccgg gccccaccag cgagacaccg | | |
| | 1621 gagctggcta cggagcagaa gcctatccag | | |
| | gaccctcagc ccacgccttc tgcaccagcc | | |
| | 1681 atgggggctg ctgaccagct agcatctgca | | |
| | agggaggcct ctcaggaatt gcctcctggc | | |
| | 1741 tttctctaca aggtggaaac actgcatgat | | |
| | tttgaggcag caaattctga tgaacttacc | | |
| | 1801 ttacaaaggg gtgatgtggt gctggtggtc | | |
| | ccctcagatt cagaagctga tcaggatgca | | |
| | 1861 ggctggctgg tgggagtgaa ggaatcagac | | |
| | tggcttcagt acagagacct tgccacctac | | |
| | 1921 aaaggcctct ttccagagaa cttcacccga | | |
| | cgcttagatt ag | | |

C.

| | | | Seq |
|---|---|---|---|
| Protein | | Accession | ID |
| [Species] | Amino Acid Sequence | No. | No. |
| keratin 18, | 1 stfstnyrsl gsvqapsyga | S06889 | Seq |
| cytoskeletal | rpvssaasvy agaggsgsri | | ID |
| [Homo sapiens] | svsrstsfrg gmgsgglatg | | No. |
| | 61 iagglagmgg iqneketmqs | | 9 |
| | lndrlasyld rvrsletenr | | |
| | rleskirehl ekkgpqvrdw | | |
| | 121 shyfkiiedl raqifantvd | | |
| | narivlqidn arlaaddfrv | | |
| | kyetelamrq svendihglr | | |
| | 181 kviddtnitr lqleteieal | | |
| | keellfmkkn heeevkglqa | | |
| | qiassgltve vdapksqdla | | |
| | 241 kimadiraqy delarknree | | |
| | ldkywsqqie esttvvttqs | | |
| | aevgaaettl telrrtvqsl | | |
| | 301 eidldsmrnl kaslenslre | | |
| | vearyalqme qlngillhle | | |
| | selaqtraeg qrqaqeyeal | | |
| | 361 lnikvkleae iatyrrlled | | |
| | gedfnlgdal dssnsmqtiq | | |
| | ktttrrivdg kvvsetndtk | | |
| | 421 vlrh | | |

D.

| | | | Seq |
|---|---|---|---|
| Protein | | Accession | ID |
| [Species] | Amino Acid Sequence | No. | No. |
| cytokeratin 8 | 1 msirvtqksy kvstsgpraf | JS0487 | Seq |
| (version 1) | ssrsytsgpg srissssfsr | | ID |
| [Homo sapiens] | vgssnfrggl gggyggasgm | | No. |
| | 61 ggitavtvnq sllsplvlev | | 10 |
| | dpniqavrtq ekeqiktlnn | | |
| | kfasfidkvr fleqqnkmle | | |
| | 121 tkwsllqqqk tarsnmdnmf | | |
| | esyinnlrrq letlgqeklk | | |
| | leaelgnmqg lvedfknkye | | |
| | 181 deinkrteme nefvlikkdv | | |
| | deaymnkvel esrlegltde | | |
| | inflrqlyee eirelqsqis | | |
| | 241 dtsvvlsmdn srsldmdsii | | |
| | aevkaqyedi anrsraeaes | | |
| | myqikyeelq slagkhgddl | | |
| | 301 rrtkteisem nrnisrlqae | | |
| | ieglkgqras leaaiadaeq | | |
| | rgelaikdan aklseleaal | | |
| | 361 qrakqdmarq lreyqelmnv | | |
| | klaldieiat yrkllegees | | |
| | rlesgmqnms ihtkttggya | | |
| | 421 gglssayggs qaglsyslgs | | |
| | sfgsgagsss fsrtsssrav | | |
| | vvkkietrdg klvsessdvl | | |

* The gene sequence for Human 78 kdalton glucose-regulated protein (GRP78) gene [Homo sapiens] can be found at Genbank accession No. M19645

### Example 37

### Cancer Cell Binding Kinetics and Localization of Chelates

Cell Lines and Reagents: Eu(III) N-(6-methyl-2-quinolylmethyl)-N′,N″,N′″-tris(methylene phosphonic acid ethyl ester)-1,4,7,10-tetraazacyclododecane (EuQCTME, from The Dow Chemical Company, USA) was prepared as a 2 mM aqueous solution by heating to 90° C. for 1 h to solubilize the compound. Human colon carcinoma (CaCo-2), head and neck carcinoma (HLaC), prostate carcinoma (DU-145), cervical carcinoma (C-33A), and non-small cell lung carcinoma (SK-MES) cell lines were purchased from American Type Cell Collection (ATCC; Rockville, Md., USA). All neoplastic cell lines were cultured in RPMI medium (Nova Tech, Grand Island, N.Y., USA) containing 10% fetal bovine serum (FBS; Nova Tech, USA). The non-neoplastic human colon epithelial line NCM460 was purchased from INCELL (San Antonio, Tex., USA) and cultured in M3:10A medium (INCELL) according to the supplier's instructions. The cells were propagated at 37° C. in a humidified atmosphere containing 5% CO₂.

Cells were counted and plated in NUNCLON 96-well cell culture white plates (Nalge Nunc International, Denmark) at a density of 10⁴ cells/well. Cells were allowed to attach in incubator (37° C., 5% CO₂ environment) over an at least 24 h period before treatment with EuQCTME. Cells were incubated in the presence of 100 μL of EuQCTME (1 mM final concentration) for 0, 2, 5, 15, 30, 45, 60, 120, 180, and 240 minutes (at t=0, no EuQCTME was added). After incubation, cells were gently washed 3× with 10 mM HEPES, 150 mM NaCl, 5.6 mM KCl, 0.7 mM Na₂HPO₄, pH 7.4.

Measurements were performed using a SPECTRAFLUOR PLUS fluorescent microplate reader (Tecan, Research Triangle Park, N.C., USA). A standard curve of the EuQCTME alone (no cells) was generated ranging from 2 mM to 0.2 pM to determine the optimal gain setting to measure bound fluorescence within the cells. Based on the above measurement, 7 dilutions in the range of 1953 nM to 15.26 nM were used for EuQCTME standard curve determinations. The following settings were optimized for measurement: excitation at 320 nm (band width: 35 nm), emission at 595 nm (band width: 35 nm), gain 110, lag time 1 μs, integration time 2000 μs, number of flashes: 1.

After measurements were gathered, HEPES buffer was gently aspirated and the cells were lysed in 100 μL of osmolytic lysis buffer (10 mM Tris, pH 7.4, 0.3% SDS) with 1% protease inhibitor cocktail (Sigma catalog number P8430, with AESBF, pepstatin A, leupeptin, E-64, and aprotinin), 1% phosphatase inhibitor cocktail (Sigma catalog number P2850, with microcystin LR, cantharidin, and p-bromotetramisole), and 1/10 volume of nuclease stock solution (50 mM MgCl₂, 100 mM Tris pH 7.0, 500 μg/mL RNase A, 100 μg/mL DNase type II). A 1:50 dilution of the cell lysate was made and measured via BCA assay (Pierce, Rockford, Ill., USA), with BSA used as a standard to determine protein concentration. Each time point measurement above (in RFU) was normalized to its corresponding protein content and recalculated as RFU/μg protein. The experiments were repeated 8 times. Each assay was repeated in triplicate. Statistical analysis of the differences between the blank readings at time=0 (with cells only, no EuQCTME) versus the readings at t=2, 5, 15, 30, 45, 60, 120, and 240 min was assessed by paired t-test (one-tailed distribution). The p values of <0.05 were considered significant. For qualitative evaluation of the area under the curve (AUC), the plots representing mean fluorescence over 240 min were drawn to the same scale, printed, and then the corresponding areas were cut out and weighed.

Binding kinetics of EuQCTME in CaCo-2 cells was compared to binding in further malignant cell lines (Du-145, SK-MES, HLaC, and C33-A) and to binding in the non-malignant cell line NCM460. These experiments were performed in cells exposed to EuQCTME for up to 240 min, gentle washing of the unbound reagent, and assessment of the bound fluorescence. The results of a representative binding experiment, showing average fluorescence of triplicate samples (Table 12) are expressed as relative fluorescence per well.

TABLE 12
EuQCTME Cell Binding (fluorescence per well)

| Time | Cell Lines | | | | | |
|---|---|---|---|---|---|---|
| (min) | C33-A | SK-MES | NCM-460 | DU145 | HLAC | Caco-2 |
| 0 | −54 | 1,020 | 895 | 149 | 771 | 1,839 |
| 2 | 2,967 | 21,983 | 2,757 | 27,884 | 15,807 | 4,708 |
| 5 | 22,763 | 11,756 | 3,325 | 10,040 | 20,493 | 3,391 |
| 15 | 5,075 | 8,588 | 14,042 | 747 | 13,704 | 6,806 |
| 30 | 2,923 | 7,237 | 18,535 | 19,868 | 6,300 | 11,168 |
| 45 | 7,030 | 9,513 | 18,737 | 10,987 | 10,068 | 10,872 |
| 60 | 583 | 5,358 | 20,666 | 3,235 | 3,206 | 21,176 |
| 120 | 1,275 | 8,159 | 2,388 | 15,644 | 21,877 | 29,389 |
| 180 | 2,377 | 5,682 | 1,598 | 9,038 | 6,510 | 31,510 |
| 240 | 1,322 | 15,625 | 16,682 | 2,506 | 2,401 | 4,575 |
| Totals | 9 | 28 | 44 | 49 | 51 | 100 |

Total - Normalized total area under each curve (AUC)

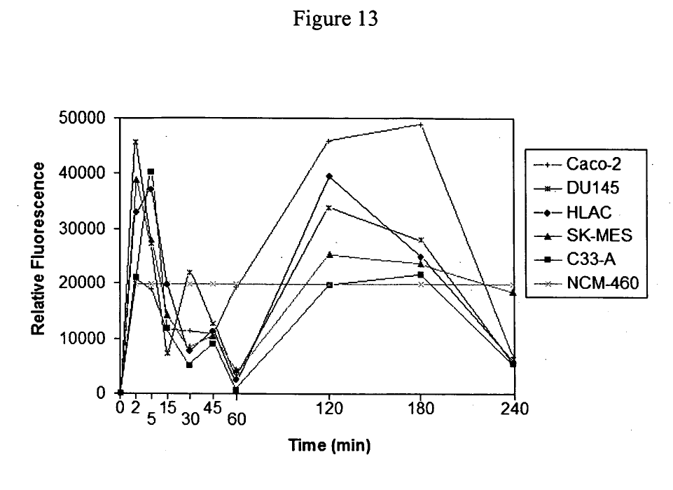

These data were then normalized to the results for NCM-460 control cells. The kinetics of binding/uptake in all of the cancer cell lines were found to be significantly different from the pattern of binding/uptake in NCM-460 control cells. As shown in FIG. 13, in the cancer cell lines, fluorescence of the cell-bound EuQCTME exhibited an initial burst within 5 minutes, and a second peak within 2 to 3 hours, relative to control cells.

Statistical analysis of the fluorescence measured in the cells exposed to EuQCTME in comparison with the untreated cells indicated that the differences in the signal were significantly greater in the cells incubated with EuQCTME (p<0.05). Qualitative assessment of the area under the curve (AUC) has shown the highest extent of EuQCTME in CaCo-2 cells, consistent with data from prior studies (not shown). The AUC followed the order: CaCo-2>(HLaC, DU-145, NCM460)>SKMES>C33A. These binding trends are representative of at least three independent experiments.

Similar data were obtained when the fluorescence was normalized per total protein (data not shown). This suggests that the protein content was comparable at all time points, as could be expected since the same numbers of cells were plated per each well. A total fluorescence (sum of signals from the membrane-bound EuQCTME interacting with the membranes in a specific and non-specific manner, plus the signal from the internalized compound) showed some variation that could result from non-specific binding of the chelate that was not fully removed by gentle washes of the cells.

### Example 38

### Subcellular Localization of Chelates

Cells were counted and plated in a 20×100 mm cell culture dish at a density of about 2×10⁶ cells. Cells were allowed to attach to the dish in an incubator (37° C., 5% CO₂ environment) over an at least 24 hr period before treatment with EuQCTME. Cells were incubated in the presence of 5 mL of EuQCTME (1 mM final concentration) for the following duration: 0, 15, 30, 60, and 240 minutes (at t=0, no EuQCTME was added).

After incubation cells were collected by trypsinization and gently centrifuged (about 2000×g) for 10 minutes. The cell supematant was discarded, and the resulting cell pellet was washed in cold PBS (phosphate buffered saline). The cell pellet was centrifuged again (about 2000 rpm) for 10 minutes. The PBS supernatant was discarded and the cold PBS wash was repeated and centrifuged as above. After discarding the PBS supernatant, the resulting cell pellet was used for NE-PER extraction (Pierce, Rockford, Ill.) according to the manufacturer's instructions.

The resulting nuclear and cytoplasmic fractions from each time point were then placed into the wells of a 96-well white plate. Due to the volume of lysate available for measurement, the fractions were diluted 1:4 in HEPES binding buffer for a total volume of 100 liL. Measurements were performed using a SPECTRAFLUOR PLUS fluorescent microplate reader (Tecan, Research Triangle Park, N.C., USA). A standard curve of the EuQCTME alone (no lysate) alone was generated ranging from 2 mM to 0.2 pM in order to determine the optimal gain setting to measure bound fluorescence within the cells. Based on the above measurement, 7 dilutions in the range of 1953 nM to 15.26 nM were used for EuQCTME standard curve determinations. The following settings were optimized for measurement: excitation at 320 nm (band width: 35 nm), emission at 595 nm (bandwidth: 35 nm), gain 110, lag time 1 μs, integration time 2000 μs, # of flashes: 1.

Because lysate volume was in limited quantity and protein concentration was relatively low, absorbances for protein measurements were read using the formula of Kalckar (1.45 OD₂₈₀-0.74OD₂₆₀=mg protein/mL). Each time point measurement above (in RFU) was normalized to its corresponding protein content and recalculated as RFU/μg protein.

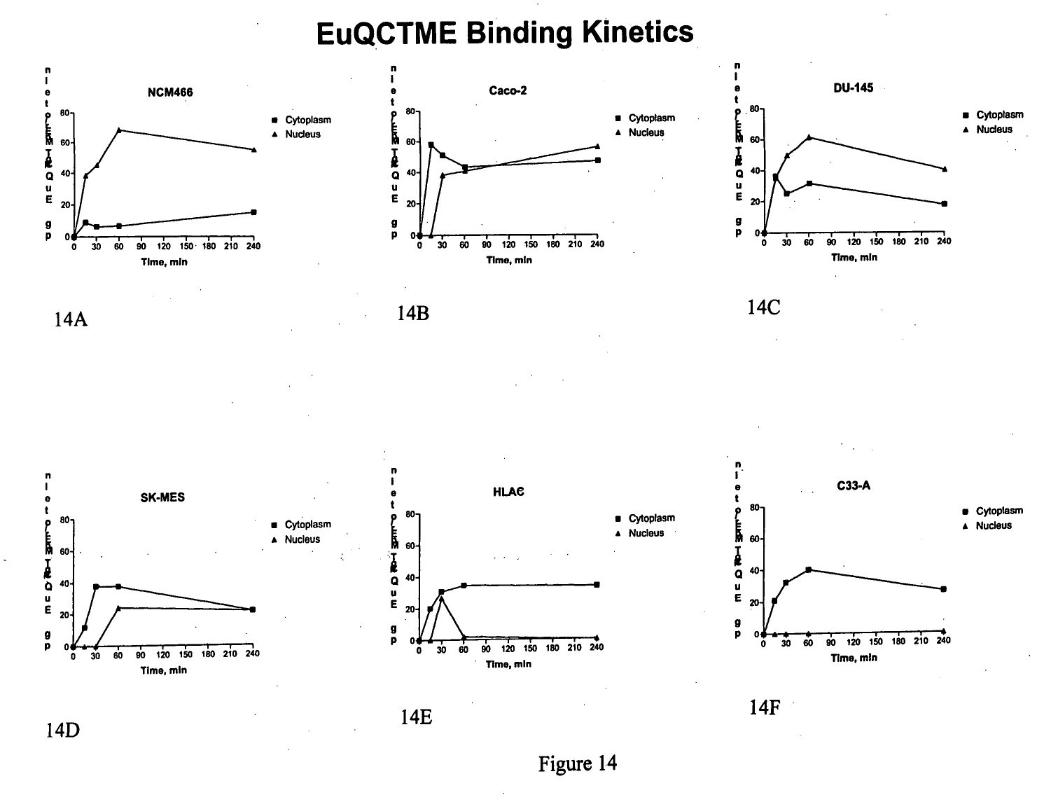

To gain further insight into uptake and subcellular localization of EuQCTME in target cells, a similar binding experiment was performed to that described above but instead of measuring the fluorescence associated with intact cells, the cells were disrupted at indicated time points and the levels of EuQCTME in the cytoplasm and nuclear fractions determined by fluorescence and quantified against a cell-free standard. As shown in FIG. 14, the maximum fluorescence signal (associated with either nucleus or cytoplasm) was comparable in all tested cancer cell types. In most cases the fluorescence rapidly reached a plateau (>1 hour). Strikingly, only the non-malignant cells NCM460 had a high nuclear signal, but very low cytoplasm-associated fluorescence (<10 pg EuQCTME per μg protein). In contrast, malignant cells had much higher cytoplasmic binding of EuQCTME, possibly due to chelate association with unique tumor-specific targets. In three malignant cells lines, CaCo-2, DU-145, and SK-MES, nuclear and cytoplasmic fluorescence was comparable up to 240 min, reaching approximately 40 pg EuQCTME per μg protein. In these cells, the nuclear signal was either very low (C33-A) or rapidly declined after an apparent initial burst (HLaC). A lesser amount of cytoplasm- and nucleus-associated fluorescence, in comparison with the total fluorescence seen in the binding kinetics experiments, is consistent with only a small fraction of the EuQCTME being internalized in the cells, with the balance of the cell-associated chelate remaining bound to or within the cell membrane.

### Example 39

### Ex-Vivo Interactions of Eu-QCTME with Excised Human Colorectal Tissue

An aqueous solution of Eu-QCTME (2 mM/5% ethanol) was prepared for topical application.

Excised tissue was received from human patients undergoing radical resection of the colon as a result of invasive cancer. Sections of tissue were received within 30 minutes of resection and the chelate solution applied as an aerosol spray followed by sequential aqueous rinsing to remove unbound chelate. The resulting tissue was then visualized under UV irradiation (320 nm) to detect the presence of the chelate (red-shifted emission). In this fashion, Eu-QCTME was found to localize in diseased tissue (adenocarcinoma and pre-malignant neoplasm) with no detectable localization in adjacent normal tissue.

### Example 40

### Chelate Localization Patterns

Direct observation of EuQCTME interactions with CaCo-2 and NCM460 cells was performed by confocal microscopy. Before images were captured, an aliquot of the HEPES binding buffer was mounted on a slide and observed for any background fluorescence. Similarly, an aliquot of the EuQCTME 2 mM stock was viewed on a separate slide. The slide with HEPES buffer alone displayed a black background, however, the image of EuQCTME was completely white indicating strong fluorescent signal (data not shown). Based on the results of the uptake kinetics studies (described above) 1 h was chosen as the incubation time since this reflects maximum EuQCTME binding in NCM460 cells. A 1 mM EuQCTME concentration was used for consistency, since this is the same final drug concentration used in the binding and subcellular localization experiments.

Confocal microscopy confirmed predominantly cytoplasmic localization of EuQCTME and association with cytoskeleton (possibly microtubules) in CaCo-2, but only a minimal uptake in NCM460. In summary, the therapeutic index indicated significant specificity of EuQCTME for malignant cells. The chelate was shown to bind rapidly to human malignant and non-malignant cells (minutes) and the binding was paralleled by rapid intracellular uptake of the drug to the nucleus and cytoplasm. Unlike the non-malignant normal cells, tumor cells showed consistently higher drug levels in the cytoplasm versus the nucleus. Confocal microscopy confirmed predominantly cytoplasmic localization of EuQCTME and association with microtubules in carcinoma cells.

### Example 41

### Growth Inhibitory Effect and Cytotoxicity of Chelates

To assess whether or not chelate treatment of cancer cells could produce a net modulation in cell growth, the overall growth inhibitory effect of chelates on various cancer cell lines was tested. The concentration of chelate necessary to produce 50% growth inhibition was measured, as determined by detection of the formazan product of cellular degradation of 3-(4,5-dimethylthiazol-2-yl)-5-(3-carboxymethoxyphenyl)-2-(4-sulfophenyl)-2H-tetrazolium, inner salt (MTS).

The chelate EuQCTME (“EuQM”) was dissolved in water at a concentration of 1.19 mM. Cultures of the ten human cancer cell and one normal cell lines listed in Table 13 were, after plating for 24 hours, treated with EuQCTME at 0 (vehicle, media), 0.006, 0.032, 0.16, 0.8, 4, 20, 100, and 500 μM, i.e. 0, 0.001, 0.006, 0.032, 0.16, 0.8, 4, 20, and 100 μg/mL. The concentration of EuQCTME required for 50% growth inhibition (IC₅₀) was calculated from the data. Results are shown below.

TABLE 13
Chelate Concentration Producing 50%
Inhibition of Cancer Cell Growth

| Cell Line | Description | EuQCTME |
|---|---|---|
| HT-29 | Colon adenocarcinoma | 15.8 μM |
| HLaC | Head & Neck squamous cell carcinoma | 21.9 |
| SK-MES | Lung, non-small cell squamous carcinoma | 140.3 |
| C-33A | Cervical, epithelial | 41 |
| LnCaP | Prostate adenocarcinoma, | 46.4 |
| | androgen-dependent | |
| DU-145 | Prostate adenocarcinoma, androgen | 54.4 |
| | independent | |
| MDA-231 | Breast adenocarcinoma | 39.6 |
| MDA-231-M | Breast adenocarcinoma, metastatic clone | 46.8 |
| | of MDA-231 | |
| Caki-1 | Renal cell carcinoma, fast growing | 16 |
| CaCo-2 | Colorectal adenocarcinoma | 133.9 |
| NCM460 | Normal human colon epithelial cells | 123.7 |
| | (control line) | |

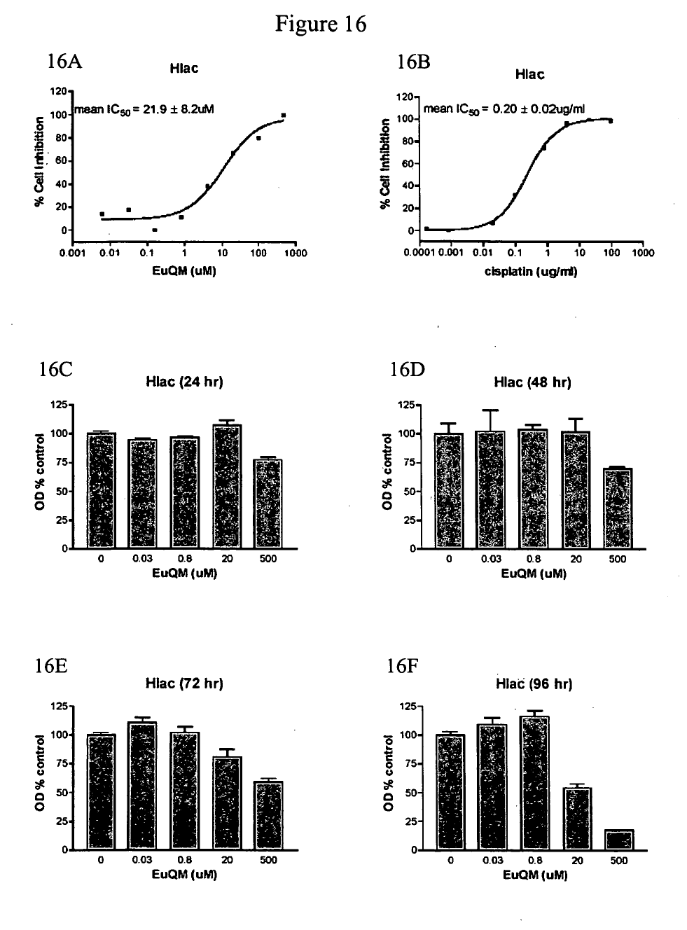

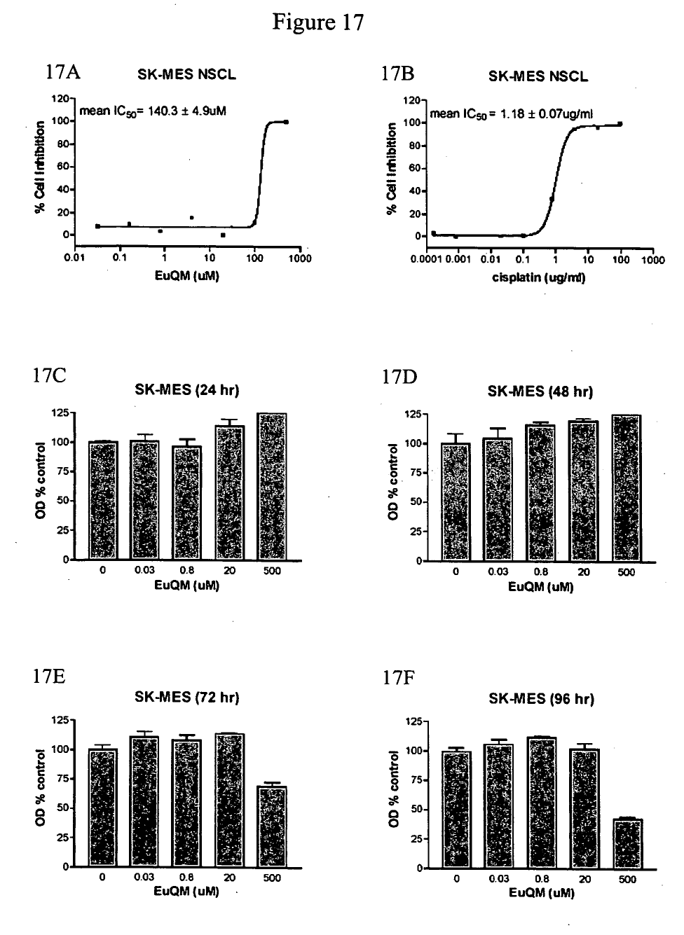

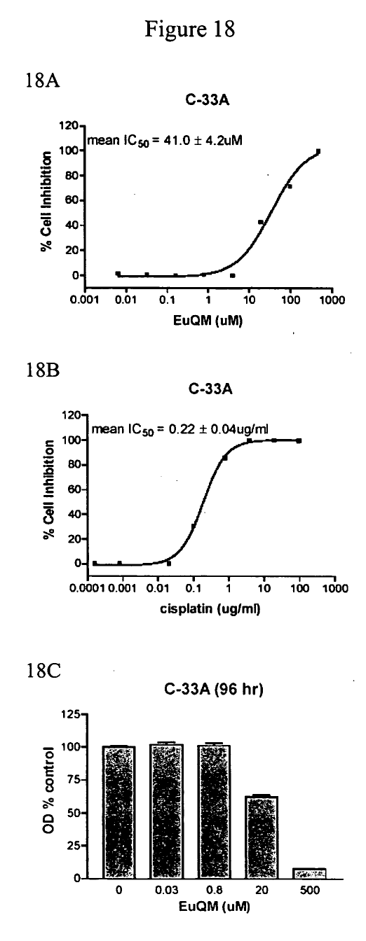

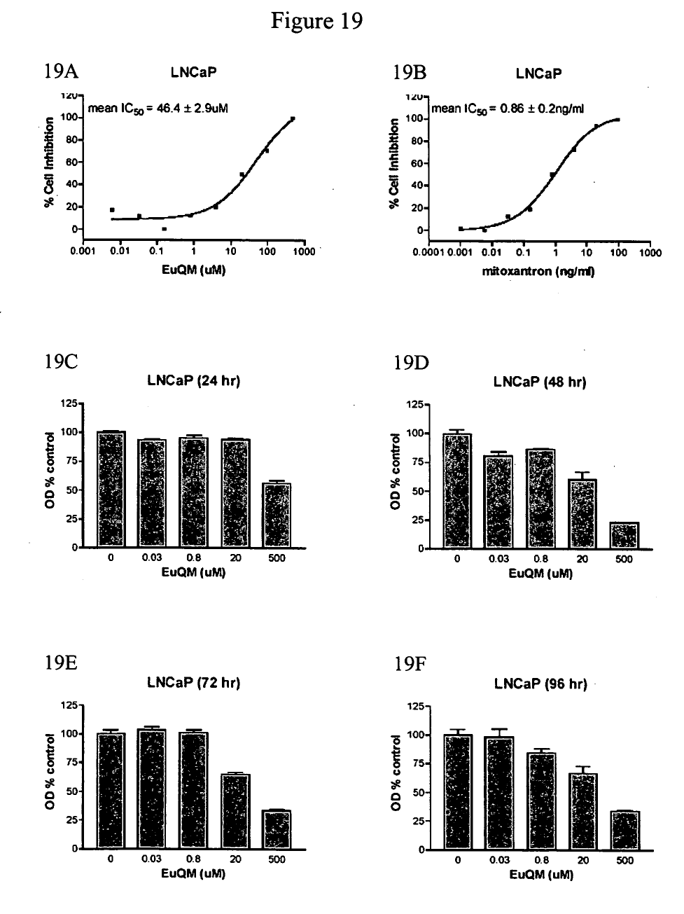

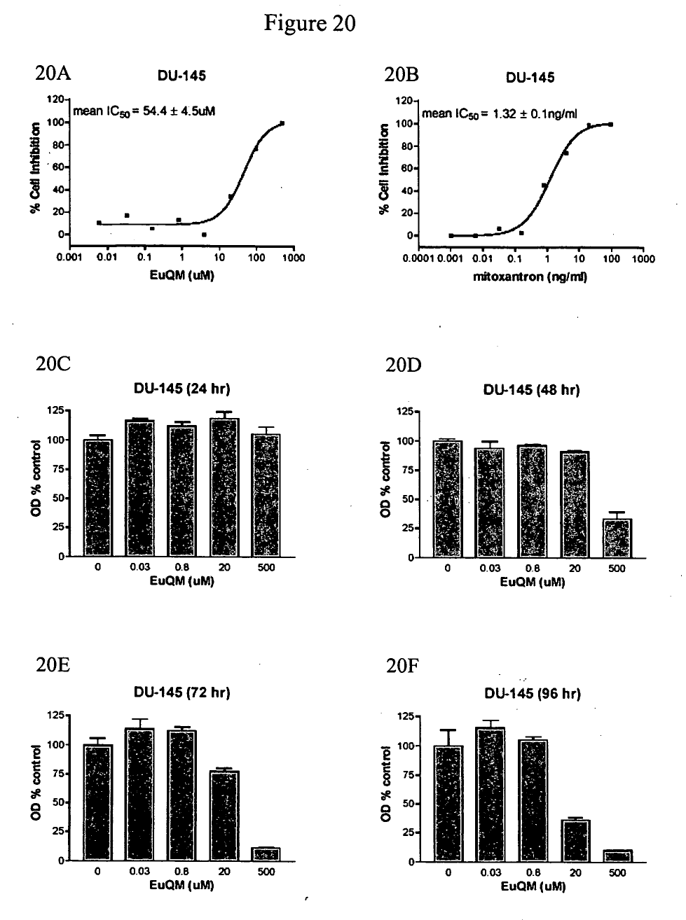

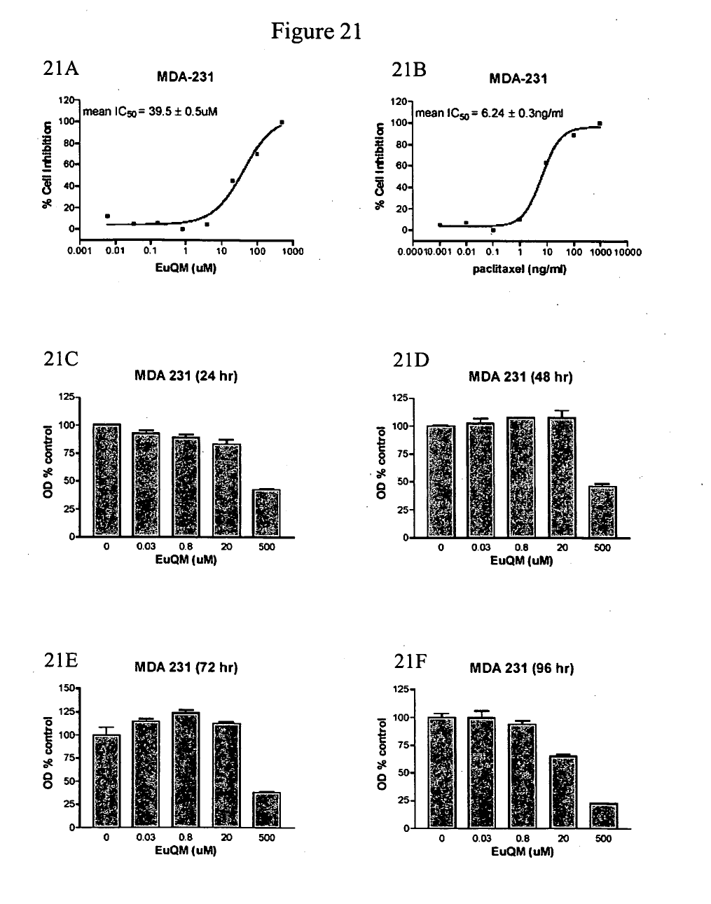

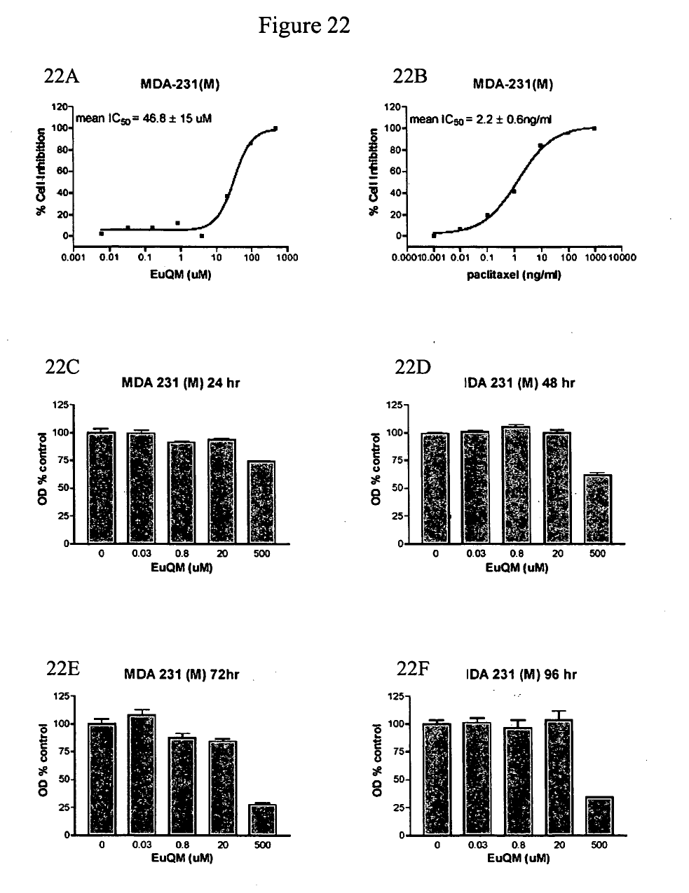

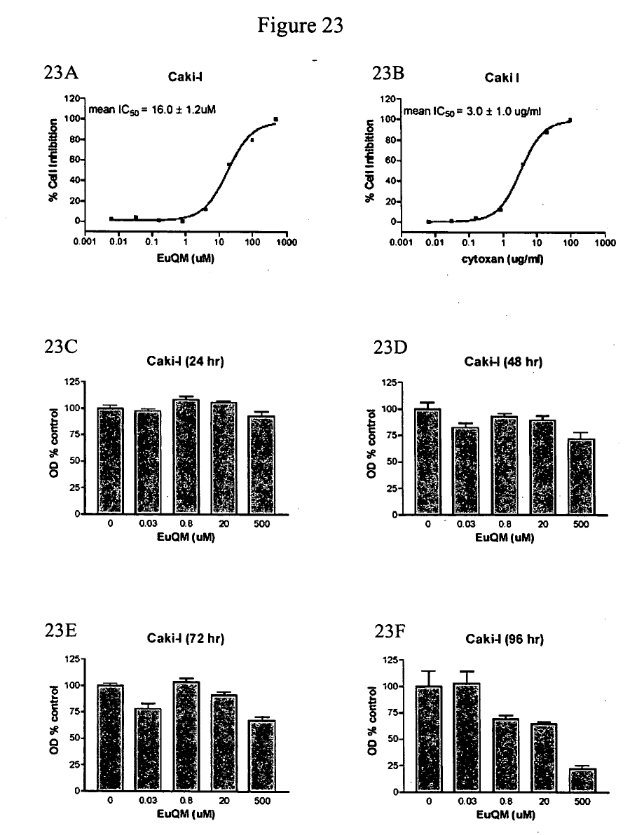

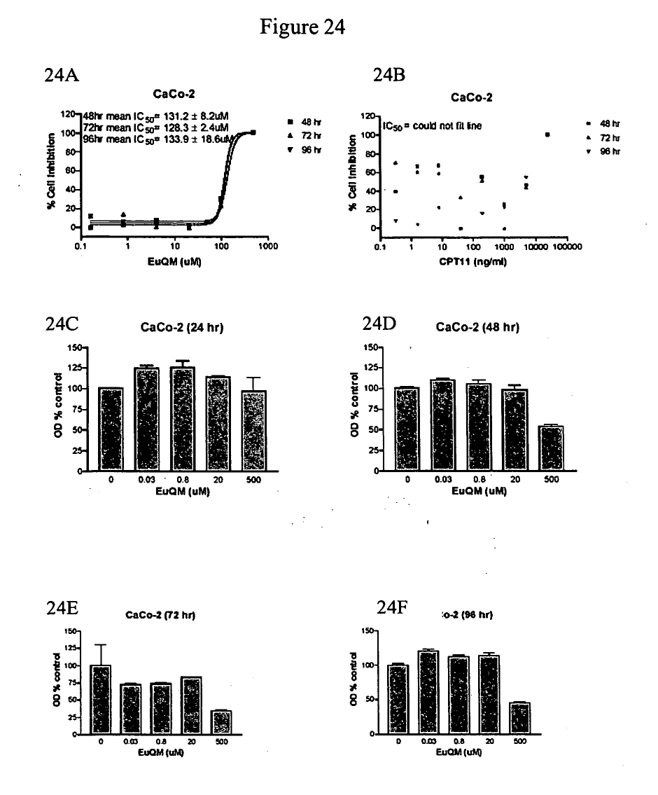

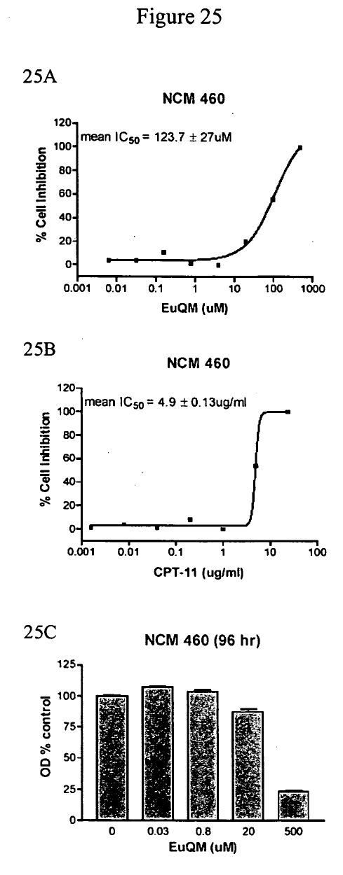

As compared to control, this data shows that growth of 8 of 10 cancer cell lines were significantly inhibited by the chelate. Time course cytotoxicity measurements are illustrated by the bar graphs in FIGS. 14-25. These indicate lack of measurable cytotoxicity of EuQM (CTME) up to 48 hours in most malignant cell lines. Breast (MDA-231) and prostate (DU-145) cancer cell lines showed inhibitory activity at the highest EuQCTME concentration (500 μM) even at the earliest time points of 24 hours. This indicates that chelates according to the present invention can be used to inhibit cancer cell growth, and can exert such an inhibitory effect when used for diagnostic and/or therapeutic purposes.

### Example 42

### Comparative Chelate Cytotoxicity

In addition, comparative cytotoxicity studies were performed, wherein the effect of EuQCTME was compared with the effect of a standard anti-cancer compound known effective against each of the cell lines. Standard anti-cancer compounds were prepared as follows. CPT-11 (camptothecin-11 or irinotecan, i.e. (4S)-4,11-diethyl-4-hydroxy-9-[(4-piperidinopiperidino)carbonyloxy]-1H-pyrano[3′,4′:6,7]indolizino[1,2-b]quinoline-3,14(4H,12H) dione hydrochloride trihydrate) was obtained from CTRC IDS (lot #29HPA) and dissolved in sodium chloride. This standard agent was used as a control for the colon tumor cell line, HT-29. CPT-11 was evaluated at 0 (vehicle, media), 0.001, 0.006, 0.032, 0.16, 0.8, 4, 20 and 100 μg/mL. Cisplatin (platinol, i.e. cis-diaminedichloroplatinum(II)) was purchased from Sigma-Aldrich (St. Louis, Mo.) and was dissolved in water. This drug was used as the positive control for the head and neck, non-small cell carcinoma, and cervical carcinoma tumor cell lines. Cisplatin was evaluated at 0 (vehicle, media), 0.001, 0.006, 0.032, 0.16, 0.8, 4, 20 and 100 μg/mL.

Mitoxantrone (mitoxantron, i.e. 1,4-dihydroxy-5,8-bis[[2-[(2-hydroxyethyl)amino]ethyl]amino]-9,10-anthracenedione dihydrochloride) was purchased from Sigma-Aldrich (St. Louis, Mo.) and was dissolved in water. The prostate carcinoma cell lines LnCaP and DU-145 were analyzed with this agent as the positive control. Mitoxantrone was evaluated at 0 (vehicle, media), 0.001, 0.006, 0.032, 0.16, 0.8, 4, 20 and 100 ng/mL. Paclitaxel (taxol, i.e. 5β, 20-Epoxy-1,2a,4,7β,10β,13a-hexahydroxytax-11-en-9-one 4,10-diacetate 2-benzoate 13-ester with (2R,3S)-N-benzoyl-3-phenylisoserine) was purchased from Sigrna-Aldrich (St. Louis, Mo.) was dissolved in 100% dimethyl sulfoxide (DMSO) to prepare a 1000× stock solution. Paclitaxel was used as the positive control with the breast carcinoma cell line MDA-231 and the metastatic clone MDA-231-M. Paclitaxel was evaluated at 0, (vehicle, 0.1% DMSO), 0.001, 0.01, 0.1, 1, 10, 100, and 1000 ng/ml. Cytoxan (cyclophosphamide, i.e. 2-[bis(2-chloroethyl)amino]tetrahydro-2H-13,2-oxazaphosphorine 2-oxide monohydrate) was obtained from the CTRC IDS (lot #3A66952) and dissolved in water. This standard agent was used as a positive control for the Caki-1 tumor cells. Cytoxan was tested at 0 (vehicle, media), 0.001, 0.006, 0.032, 0.16, 0.8, 4, 20 and 100 μg/mL.

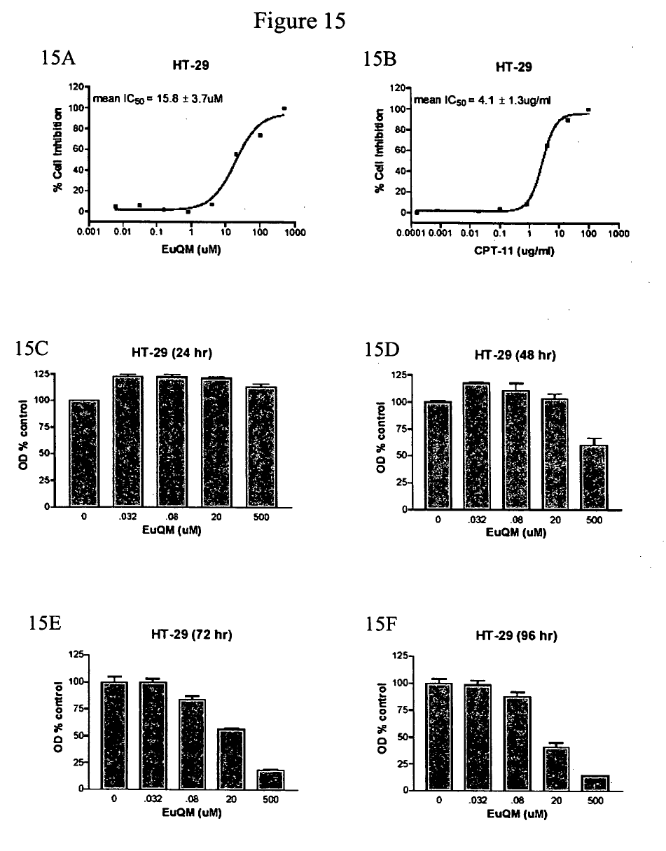

Comparison of EuQCTME with standard drug cytotoxicity is summarized in FIGS. 15-25. Representative growth inhibition curves determined at 96 hours continuous exposure to the drugs are shown in FIGS. 15A-15B through 25A-25B. The mean IC₅₀ values computed in each cell line for EuQCTME and the standard agents is also shown.

As shown in this example, EuQCTME was found to have a novel cytotoxic activity. This chelate, at 500 μM, showed over 30% growth inhibition in MDA-MB-231 and LNCAP at 24 hours exposure. Half-maximum growth inhibition was noted in examined cancer cell lines at 16-140 μM concentration levels with a 96 hour continuous exposure. EuQCTME showed a nearly ten-fold range of cytotoxicity in human neoplastic cell lines representing colon, head and neck, lung, prostate, cervical, breast, and renal carcinomas spanning a range from 16 μM to 140 μM with a 96 hour continuous exposure. Relatively low drug cytotoxicity (123 μM) was observed in the non-neoplastic normal cell line NCM460. Thus, targeting chelates according to the present invention have been found to exhibit unexpected tumor cell inhibitory activity and appear reasonably non-toxic toward healthy cells, as compared with the effects of standard anti-cancer drugs.

The invention will be further understood by the following non-limiting claims.

What is claimed is:

1. An aminophosphonic acid compound of the Formula (I): 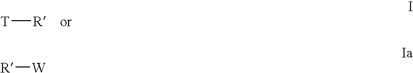
wherein T is 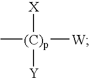
wherein p is 0, 1, 2, 3, 4, 5 or 6;
wherein each X and Y if present is independently H, OH, C₁-C₆ alkyl, substituted or unsubstituted aryl, or unsubstituted or substituted heterocycle;
wherein W is 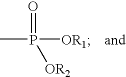
wherein each R₁ and R₂ are independently H or C₁-C₆ linear or branched alkyl, or C₁-C₆ linear or branched alkenyl or aryl, and optionally are methyl, ethyl, propyl or butyl with the proviso that at least one of R₁ or R₂ is H; and
wherein R is 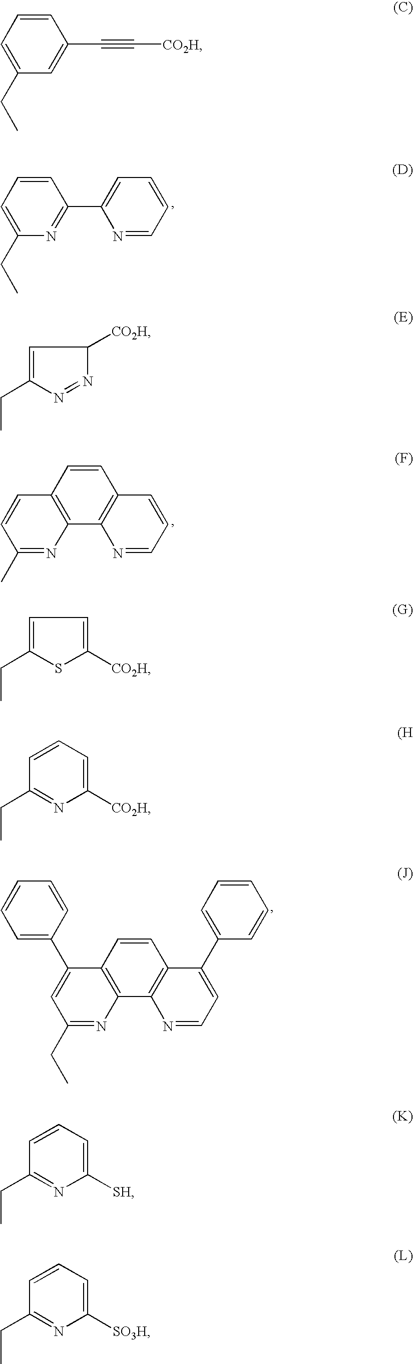 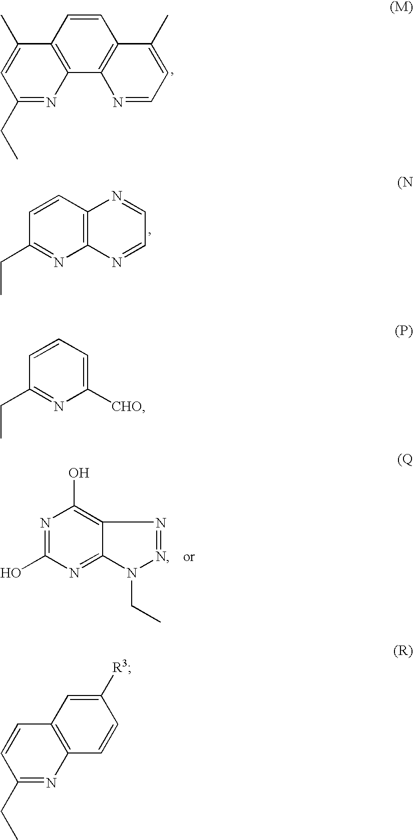
wherein R₃ is H or C₁-C₆ alkyl.

2. The compound of claim 1, wherein p is 0, 1 or 2, and X and Y are both H.

3. The compound of claim 1, wherein T is CH₂PO(OR₁)OR₂ wherein R₁ is H and R₂ is C₂-C₆ alkyl.

4. The compound of claim 2 wherein R₁ is H and R₂ is C₄ alkyl, and R′ is (R) wherein R³ is CH₃.

5. The compound of claim 1 of Formula Ia:
wherein one of R₁ and R₂ is H and the other of R₁ and R₂ is ethyl, propyl, isopropyl, cyclopropyl, n-butyl, sec-butyl, tert-butyl, isobutyl, cyclobutyl, or pentyl; and
wherein R′ is: 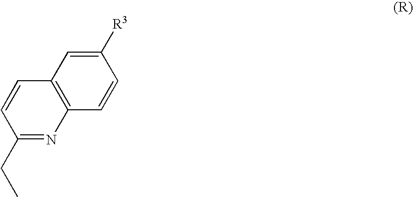
wherein R³ is methyl, ethyl or propyl.

6. A polyaminophosphonic acid compound of Formula (II): 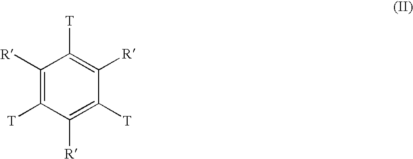
wherein each T is independently 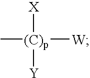
wherein p is 0, 1, 2, 3, 4, 5 or 6;
wherein each X and Y are independently H, OH, C₁-C₆ alkyl, substituted or unsubstituted aryl, or unsubstituted or substituted heterocycle;
wherein each W is independently 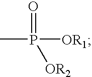
wherein each R₁ and R₂ are independently H, C₂-C₁₀ linear or branched alkyl, C₂-C₁₀ linear or branched alkenyl, C₂-C₁₀ linear or branched alkynyl, trifluoromethyl, substituted or unsubstituted aryl, or substituted or unsubstituted heteroaryl; and
wherein R′ is H or C₁-C₆ alkyl.

7. The compound of claim 6, wherein, R′ is H, T is CH₂PO(OR₁)OR₂, R₁ is H and R₂ is C₂-C₆ alkyl.

8. The compound of claim 7, wherein R₂ is n-butyl.

9. The compound of claim 6, wherein R′ is Me, T is CH₂PO(OR₁)OR₂, R₁ is H and R₂ is a C₃-C₆ alkyl.

10. The compound of claim 9, wherein R₂ is n-butyl.

11. An isolated non-covalent complex of a chelant and a polypeptide having an amino acid sequence of SEQ ID NO: 2.

12. The complex of claim 11, wherein the chelant is a polyaminophosphonic acid metal complex of Formula (V): 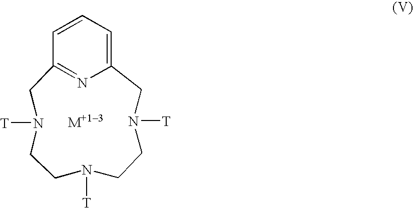
wherein each T is independently 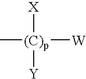
wherein p is 0, 1, 2, 3, 4, 5 or 6; and
wherein each X and Y are independently H, OH, C₁-C₆ alkyl, substituted or unsubstituted aryl, or unsubstituted or substituted heterocycle;
wherein W is 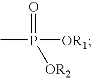
wherein each R₁ and R₂ are independently H, C₂-C₁₀ linear or branched alkyl, C₂-C₁₀ linear or branched alkenyl, C₂-C₁₀ linear or branched alkynyl, trifluoromethyl, substituted or unsubstituted aryl, or substituted or unsubstituted heteroaryl; and
wherein M is a metal cation having a valency of at least +1.

13. The complex of claim 12, wherein M is selected from the group consisting of La, Ce, Pr, Nd, Pm, Sm, Eu, Gd, Tb, Dy, Ho, Er, Tm, Yb, Y, In and Lu.

14. The complex of claim 12 wherein each T is CH₂PO(OR₁)OR₂, wherein R₁ is H, R₂ is a C₂-C₆ alkyl, and M is Tb.

15. The complex of claim 14, wherein R₂ is n-butyl.

16. A method of diagnosing or treating a disease in a host, the method comprising administering a compound of claim 1 or 6 or a salt or metal chelate thereof to abnormal cells in the host associated with a disease state.

17. A method of diagnosing or treating a disease in a host, the method comprising administering an effective amount of a salt or metal chelate of QCTME to abnormal cells in the host associated with a disease state.

18. The method of claim 17, wherein the disease state is a tumor.

19. The method of claim 17, wherein the disease state is epithelial cancer or cancer of the lymphatic system.

20. The method of claim 17, wherein the disease state is cancer of epithelial or endothelial origin.

21. The method of claim 17 wherein the disease state is cancer of the skin, colon, oral, esophagus, cervical, prostate, leukemia, liver, or breast.

22. The method of claim 16, comprising a method of diagnosing a tumor in a host, wherein the tumors are detected ex-vivo, in-vitro or in vivo.

23. A method of diagnosing or treating a disease comprising administering to a host or tissue or cell sample a compound capable of binding to a protein having the sequence of SEQ. ID NO.:2.

24. A pharmaceutical composition comprising a compound of claim 1 or 6 and a pharmaceutically acceptable carrier.


SEQUENCE LISTING (16 sequences)

SEQ ID NO: 1 - DNA, 339 bp, Homo sapiens
  CDS (1)..(339): HSPC194 (chelate binding protein)
atgcaggacactggctcagtagtgcctttgcattggtttggctttggctacgcagcactggttgcttctggtgggatcattggctatgtaaaagcaggcagcgtgccgtccctggctgcagggctgctctttggcagtctagccggcctgggtgcttaccagctgtctcaggatccaaggaacgtttgggttttcctagctacatctggtaccttggctggcattatgggaatgaggttctaccactctggaaaattcatgcctgcaggtttaattgcaggtgccagtttgctgatggtcgccaaagttggagttagtatgttcaacagaccccattag

SEQ ID NO: 2 - protein, 112 aa, Homo sapiens
MQDTGSVVPLHWFGFGYAALVASGGIIGYVKAGSVPSLAAGLLFGSLAGLGAYQLSQDPRNVWVFLATSGTLAGIMGMRFYHSGKFMPAGLIAGASLLMVAKVGVSMFNRPH

SEQ ID NO: 3 - DNA, 18 bp, Artificial Sequence
  Forward Primer for HSPC194 PCR
tggtaccttggctggcat

SEQ ID NO: 4 - DNA, 21 bp, Artificial Sequence
  Reverse Primer for HSPC194 PCR
ctaatggggtctgttgaacat

SEQ ID NO: 5 - protein, 653 aa, Homo sapiens
MKLSLVAAMLLLLSAARAEEEDKKEDVGTVVGIDLGTTYSCVGVFKNGRVEIIANDQGNRITPSYVAFTPEGERLIGDAAKNQLTSNPENTVFDAKRLIGRTWNDPSVQQDIKFLPFKVVEKKTKPYIQVDIGGGQTKTFAPEEISAMVLTKMKETAEAYLGKKVTHAVVTVPAYFNDAQRQATKDAGTIAGLNVMRIINEPTAAAIAYGLDKREGEKNILVFDLGGGTFDVSLLTIDNGVFEVVATNGDTHLGGEDFDQRVMEHFIKLYKKKTGKDVRKDNRAVQKLRREVEKAKALSSQHQARIEIESFYEGEDFSETLTRAKFEELNMDLFRSTMKPVQKVLEDSDLKKSDIDEIVLVGGSTRIPKIQQLVKEFFNGKEPSRGINPDEAVAYGAAVQAGVLSGDQDTGDLVLLHVCPLTLGIETVGGVMTKLIPSNTVVPTKNSQIFSTASDNQPTVTIKVYEGERPLTKDNHLLGTFDLTGIPPAPRGVPQIEVTFEIDVNGILRVTAEDKGTGNKNKITITNDQNRLTPEEIERMVNDAEKFAEEDKKLKERIDTRNELESYAYSLKNQIGDKEKLGGKLSSEDKETMEKAVEEKIEWLESHQDADIEDFKAKKKELEEIVQPIISKLYGSAGPPPTGEEDTAEKDEL

SEQ ID NO: 6 - DNA, 1962 bp, Homo sapiens
atgaagctctccctggtggccgcgatgctgctgctgctcagcgcggcgcgggccgaggaggaggacaagaaggaggacgtgggcacggtggtcggcatcgacttggggaccacctactcctgcgtcggcgtgttcaagaacggccgcgtggagatcatcgccaacgatcagggcaaccgcatcacgccgtcctatgtcgccttcactcctgaaggggaacgtctgattggcgatgccgccaagaaccagctcacctccaaccccgagaacacggtctttgacgccaagcggctcatcggccgcacgtggaatgacccgtctgtgcagcaggacatcaagttcttgccgttcaaggtggttgaaaagaaaactaaaccatacattcaagttgatattggaggtgggcaaacaaagacatttgctcctgaagaaatttctgccatggttctcactaaaatgaaagaaaccgctgaggcttatttgggaaagaaggttacccatgcagttgttactgtaccagcctattttaatgatgcccaacgccaagcaaccaaagacgctggaactattgctggcctaaatgttatgaggatcatcaacgagcctacggcagctgctattgcttatggcctggataagagggagggggagaagaacatcctggtgtttgacctgggtggcggaaccttcgatgtgtctcttctcaccattgacaatggtgtcttcgaagttgtggccactaatggagatactcatctgggtggagaagactttgaccagcgtgtcatggaacacttcatcaaactgtacaaaaagaagacgggcaaagatgtcaggaaggacaatagagctgtgcagaaactccggcgcgaggtagaaaaggccaaggccctgtcttctcagcatcaagcaagaattgaaattgagtccttctatgaaggagaagacttttctgagaccctgactcgggccaaatttgaagagctcaacatggatctgttccggtctactatgaagcccgtccagaaagtgttggaagattctgatttgaagaagtctgatattgatgaaattgttcttgttggtggctcgactcgaattccaaagattcagcaactggttaaagagttcttcaatggcaaggaaccatcccgtggcataaacccagatgaagctgtagcgtatggtgctgctgtccaggctggtgtgctctctggtgatcaagatacaggtgacctggtactgcttcatgtatgtccccttacacttggtattgaaactgtaggaggtgtcatgaccaaactgattccaagtaatacagtggtgcctaccaagaactctcagatcttttctacagcttctgataatcaaccaactgttacaatcaaggtctatgaaggtgaaagacccctgacaaaagacaatcatcttctgggtacatttgatctgactggaattcctcctgctcctcgtggggtcccacagattgaagtcacctttgagatagatgtgaatggtattcttcgagtgacagctgaagacaagggtacagggaacaaaaataagatcacaatcaccaatgaccagaatcgcctgacacctgaagaaatcgaaaggatggttaatgatgctgagaagtttgctgaggaagacaaaaagctgaaggagcgcattgatactagaaatgagttggaaagctatgcctattctctaaagaatcagattggagataaagaaaagctgggaggtaaactttcctctgaagataaggagaccatggaaaaagctgtagaagaaaagattgaatggctggaaagccaccaagatgctgacattgaagacttcaaagctaagaagaaggaactggaagaaattgttcaaccaattatcagcaaactctatggaagtgcaggccctcccccaactggtgaagaggatacagcagaaaaagatgagttgtag

SEQ ID NO: 7 - protein, 653 aa, Homo sapiens
MADIKTGIFAKNVQKRLNRAQEKVLQKLGKADETKDEQFEEYVQNFKRQEAEGTRLQRELRGYLAAIKGMQEASMKLTESLHEVYEPDWYGREDVKMVGEKCDVLWEDFHQKLVDGSLLTLDTYLGQFPDIKNRIAKRSRKLVDYDSARHHLEALQSSKRKDESRISKAEEEFQKAQKVFEEFNVDLQEELPSLWSRRVGFYVNTFKNVSSLEAKFHKEIAVLCHKLYEVMTKLGDQHADKAFTIQGAPSDSGPLRIAKTPSPPEEPSPLPSPTASPNHTLAPASPAPARPRSPSQTRKGPPVPPLPKVTPTKELQQENIISFFEDNFVPEISVTTPSQNEVPEVKKEETLLDLDFDPFKPEVTPAGSAGVTHSPMSQTLPWDLWTTSTDLVQPASGGSFNGFTQPQDTSLFTMQTDQSMICNLIIPGADADAAVGTLVSAAEGAPGEEAEAEKATVPAGEGVSLEEAKIGTETTEGAESAQPEAEELEATVPQEKVIPSVVIEPASNHEEEGENEITIGAEPKETTEDAAPPGPTSETPELATEQKPIQDPQPTPSAPAMGAADQLASAREASQELPPGFLYKVETLHDFEAANSDELTLQRGDVVLVVPSDSEADQDAGWLVGVKESDWLQYRDLATYKGLFPENFTRRLD

SEQ ID NO: 8 - DNA, 1962 bp, Homo sapiens
atggccgacatcaagacgggcatcttcgccaagaacgtccagaagcgactcaaccgcgcgcaggaaaaggtcctccaaaagctggggaaagctgatgagacaaaagacgaacagttcgaagaatatgtccagaacttcaaacggcaagaagcagagggtaccagacttcagcgagaactccgaggatatttagcagcaatcaaaggcatgcaggaggcctccatgaagctcacagagtcgctgcatgaagtctatgagcctgactggtatgggcgggaagatgtgaaaatggttggtgagaaatgtgatgtgctgtgggaagacttccatcaaaaactcgtggatgggtccttgctaacactggatacctacctggggcaatttcctgacataaagaatcgcatcgccaagcgcagcaggaagctagtggactatgacagtgcccgccaccatctggaagctctgcagagctccaagaggaaggatgagagtcgaatctctaaggcagaagaagaatttcagaaagcacagaaagtgtttgaagagtttaacgttgacttacaagaagagttaccatcattatggtcaagacgagttggattttatgttaatactttcaaaaacgtctccagccttgaagccaagtttcataaggaaattgcggtgctttgccacaaactgtatgaagtgatgacaaaactgggtgaccagcacgccgacaaggccttcaccatccaaggagcgcccagtgattcgggtcctctccgcattgcaaagacaccatcaccgcctgaggagccttcacccctcccgagcccgacagcaagtccaaatcatacattagcacctgcgtctcccgcaccagcacggcctcggtcaccttcacagacaaggaaagggcctcctgtcccacctctacctaaagtcaccccgacaaaggaactgcagcaggagaacatcatcagtttctttgaggacaactttgttccagaaatcagtgtgacaacaccttcccagaatgaagtccctgaggtgaagaaagaggagactttgctggatctggactttgatcctttcaagcccgaggtgacacctgcaggttctgctggagtgacccactcacccatgtctcagacattgccctgggacctatggacgacaagcactgatttggtacagccggcttctggtggttcatttaatggattcacacagccccaggatacttcattattcacaatgcagacagaccagagtatgatctgcaacttgatcatacctggagctgatgctgatgcagctgttggaaccttggtgtcagcagctgagggggccccaggagaggaagcagaggcggagaaggccactgtccctgccggggaaggagtaagtttagaggaggccaaaattggaactgaaaccactgagggtgcagagagtgcccaacctgaagcagaggagctcgaagcaacagtgcctcaggagaaggtcattccttcggtggtcatagagcctgcctccaaccatgaagaggaaggagaaaacgaaataactataggtgcagagcccaaggagaccaccgaggacgcggctcctccgggccccaccagcgagacaccggagctggctacggagcagaagcctatccaggaccctcagcccacgccttctgcaccagccatgggggctgctgaccagctagcatctgcaagggaggcctctcaggaattgcctcctggctttctctacaaggtggaaacactgcatgattttgaggcagcaaattctgatgaacttaccttacaaaggggtgatgtggtgctggtggtcccctcagattcagaagctgatcaggatgcaggctggctggtgggagtgaaggaatcagactggcttcagtacagagaccttgccacctacaaaggcctctttccagagaacttcacccgacgcttagattag

SEQ ID NO: 9 - protein, 424 aa, Homo sapiens
STFSTNYRSLGSVQAPSYGARPVSSAASVYAGAGGSGSRISVSRSTSFRGGMGSGGLATGIAGGLAGMGGIQNEKETMQSLNDRLASYLDRVRSLETENRRLESKIREHLEKKGPQVRDWSHYFKIIEDLRAQIFANTVDNARIVLQIDNARLAADDFRVKYETELAMRQSVENDIHGLRKVIDDTNITRLQLETEIEALKEELLFMKKNHEEEVKGLQAQIASSGLTVEVDAPKSQDLAKIMADIRAQYDELARKNREELDKYWSQQIEESTTVVTTQSAEVGAAETTLTELRRTVQSLEIDLDSMRNLKASLENSLREVEARYALQMEQLNGILLHLESELAQTRAEGQRQAQEYEALLNIKVKLEAEIATYRRLLEDGEDFNLGDALDSSNSMQTIQKTTTRRIVDGKVVSETNDTKVLRH

SEQ ID NO: 10 - protein, 480 aa, Homo sapiens
MSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTGGYAGGLSSAYGGSQAGLSYSLGSSFGSGAGSSSFSRTSSSRAVVVKKIETRDGKLVSESSDVL

SEQ ID NO: 11 - protein, 31 aa, Artificial Sequence
  Tryptic peptide fragment of HSPC194
MQDTGSVVPLHWFGFGYAALVASGGIIGYVK

SEQ ID NO: 12 - protein, 29 aa, Artificial Sequence
  Tryptic peptide fragment of HSPC194
AGSVPSLAAGLLFGSLAGLGAYQLSQDPR

SEQ ID NO: 13 - protein, 19 aa, Artificial Sequence
  Tryptic peptide fragment of HSPC194
NVWVFLATSGTLAGIMGMR

SEQ ID NO: 14 - protein, 6 aa, Artificial Sequence
  Tryptic peptide fragment of HSPC194
FYHSGK

SEQ ID NO: 15 - protein, 17 aa, Artificial Sequence
  Tryptic peptide fragment of HSPC194
FMPAGLIAGASLLMVAK

SEQ ID NO: 16 - protein, 10 aa, Artificial Sequence
  Tryptic peptide fragment of HSPC194
VGVSMFNRPH